(19) United States
(12) Patent Application Publication
(10) Pub. No.: US 2026/0258033 A1
(43) Pub. Date: Sep. 3, 2026

(54) 2,7-Naphthyridine Compounds as MASTL Inhibitors

(71) Applicant: Pfizer Inc., New York, NY (US)

(72) Inventors: Madeline Berry, San Diego, CA (US); Klaus Ruprecht Dress, La Jolla, CA (US); Rebecca Anne Gallego, San Diego, CA (US); Samantha Elizabeth Greasley, San Diego, CA (US); Ted William Johnson, Carlsbad, CA (US); Indrawan James McAlpine, San Diego, CA (US); Ryan Lloyd Patman, San Diego, CA (US); Romelia Del Carmen Salomon Ferrer, San Diego, CA (US); Stephanie Anne Scales, San Diego, CA (US); Michelle Bich Tran-Dubé, San Diego, CA (US); Fen Wang, San Diego, CA (US); Shouliang Yang, San Diego, CA (US)

(73) Assignee: Pfizer Inc., New York, NY (US)

(21) Appl. No.: 18/877,740

(22) PCT Filed: Jun. 28, 2023
(86) PCT No.: PCT/IB2023/056693

§ 371 Date: Dec. 20, 2024

Related U.S. Application Data
(60) Provisional application No. 63/357,834, filed on Jul. 1, 2022.

(51) Int. Cl. C07D 471/04; A61K 31/4375; A61K 31/444; A61K 31/506

(52) U.S. Cl. CPC C07D 471/04; A61K 31/4375; A61K 31/444; A61K 31/506

(57) ABSTRACT

The invention relates to compounds of Formula (1):

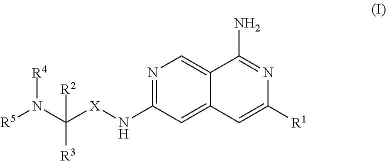

or pharmaceutically acceptable salts thereof, wherein X and R¹ to R⁵ are as defined in the description; to their use in medicine; to compositions containing them; to processes for their preparation; and to intermediates used in such processes. The compounds the present invention may inhibit the activities of MASTL receptor and may be useful in the treatment, prevention, suppression and amelioration of cancers, or diseases, disorders and conditions mediated by MASTL receptor.

### BACKGROUND OF THE INVENTION

The present invention relates to novel 2,7-naphthyridine compounds as microtubule-associated serine/threonine kinase-like (MASTL) inhibitors. The invention also relates to the preparation of the compounds and intermediates used in the preparation, compositions containing the compounds, and uses of the compounds for the treatment of MASTL related diseases such as cancers.

MASTL, also known as Greatwall kinase, is a key mitotic kinase that regulates mitotic progression and maintains mitotic integrity. MASTL plays a unique role in the control of cell cycle progression through the regulation of specific Protein Phosphatase 2A (PP2A) complexes critical for execution of mitosis via control of the mitotic CDK phospho-proteome during entry and exit from mitosis. Recent studies have suggested that MASTL acts as an oncogenic kinase by inactivating tumor suppressive PP2A-B55 complex in breast cancer cells, thereby regulating various oncogenic properties, such as cellular transformation, chromosome instability, and metastasis. MASTL targeting has reduced tumor growth in various in vitro and in vivo tumor models. Notably, MASTL is highly expressed in multiple types of cancers, including breast, head and neck, gastric, thyroid, and colorectal cancers. MASTL inhibition selectively eradicated proliferative cancer cells rather than normal cells by inducing mitotic catastrophe. Therefore, accumulating evidence indicates that MASTL may be an attractive, druggable target for selective anticancer treatment.

Although recent studies suggest that MASTL may be a promising target for selective anticancer treatment, very limited MASTL inhibitors with potent antitumor efficacy have been reported. Recently, compound 2-(4-((4-((5-cyclopropyl-1H-pyrazol-3-yl)amino)quinazolin-2-yl)amino)phenyl)acetonitrile (CAS Number 438204-56-9), also known as MKI-2, has been screened and identified as a potential MASTL inhibitor with in vitro IC₅₀ of 37.44 nM and cellular IC₅₀ of 142.7 nM in breast cancer cells. (see Kang et al., Pharmaceuticals 2021, 14, 647). This compound was previously disclosed as an inhibitor for aurora-2 protein kinase and/or glycogen synthase kinase-3. (see PCT Publication No. WO 2002/050065 A2). There is no FDA approved MASTL inhibitor yet for cancer treatment.

Accordingly, there remains a need for novel and more potent MASTL inhibitors that may be used for the treatment of MASTL related diseases such as cancers.

### SUMMARY OF THE INVENTION

The present invention provides, in part, compounds of Formula (I), and pharmaceutically acceptable salts thereof. The compounds of the present invention may inhibit the activities of MASTL, and may be useful in the treatment, prevention, suppression, and amelioration of diseases such as cancers, disorders and conditions mediated by MASTL. Also provided are pharmaceutical compositions, comprising the compounds or salts of the invention, alone or in combination with additional anticancer therapeutic agents. The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I):

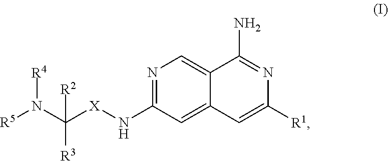

or a pharmaceutically acceptable salt thereof, wherein:
X is 5-10 membered heteroaryl comprising one, two, or three heteroatoms selected from the group consisting of O, N and S, wherein X is optionally substituted with one or two substituents independently selected from the group consisting of halogen, C₁-C₆ alkyl, C₃-C₆ cycloalkyl, C₁-C₆ fluoroalkyl, and C₁-C₆ alkoxy;
R¹ is 5-10 membered heteroaryl or 4-10 membered heterocycloalkyl, wherein R¹ comprises one, two, three, or four heteroatoms selected from the group consisting of O, N and S, and R¹ is optionally substituted with one, two, three, or four substituents independently selected from the group consisting of halogen, C₁-C₆ alkyl, C₃-C₆ cycloalkyl, C₁-C₆ fluoroalkyl, and oxo (═O);
R² and R³ are each independently selected from the group consisting of H, C₁-C₆ alkyl, C₃-C₆ cycloalkyl, C₂-C₆ alkoxyalkyl, and C₁-C₆ fluoroalkyl, or R² and R³ are taken together with the carbon atom to which they are attached to form a 3-8 membered cycloalkyl, or one of R² and R³ is taken together with the carbon atom to which they are attached to, and together with X to form a 3-8 membered ring that is fused to X; and
R⁴ and R⁵ are each independently selected from the group consisting of H, C₁-C₆ alkyl, C₃-C₆ cycloalkyl, and C₁-C₆ fluoroalkyl, or R⁴ and R⁵ are taken together with the N atom to which they are attached to form a 3-8 membered heterocycloalkyl, or one of R⁴ and
R⁵ is taken together with the N atom to which they are attached to, and together with X to form a 5-8 membered ring that is fused to X;
wherein one of R² and R³ with the carbon atom to which they are attached to, and one of R⁴ and R⁵ with the N atom to which they are attached to, are optionally taken together to form a 3-8 membered heterocycloalkyl.

Described below are embodiments of the invention, where for convenience Embodiment 1 (E1) is identical to the embodiment of Formula (I) provided above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.
E1 A compound of Formula (I) or a pharmaceutically acceptable salt thereof, as defined above.
E2 A compound of embodiment E1, or a pharmaceutically acceptable salt thereof, wherein X is 6-membered heteroaryl comprising one or two N atoms.
E3 A compound of embodiment E2, or a pharmaceutically acceptable salt thereof, wherein X is pyridinyl or pyrimidinyl.
E4 A compound of embodiment E3, or a pharmaceutically acceptable salt thereof, wherein X is pyridinyl.
E5 A compound of any one of embodiments E1 to E4, or a pharmaceutically acceptable salt thereof, wherein the compound has Formula (I-a):

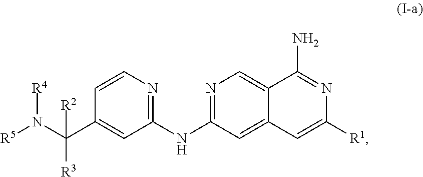

E6 A compound of any one of embodiments E1 to E5, or a pharmaceutically acceptable salt thereof, wherein R¹ is 5-8 membered heteroaryl or 5-6 membered heterocycloalkyl comprising one, two, or three heteroatoms selected from the group consisting of O, N and S, wherein R¹ is optionally substituted with one or two substituents independently selected from the group consisting of halogen, C₁-C₃ alkyl, C₃-C₆ cycloalkyl, C₁-C₃ fluoroalkyl, and oxo (═O).
E7 A compound of embodiment E6, or a pharmaceutically acceptable salt thereof, wherein R¹ is selected from the group consisting of:

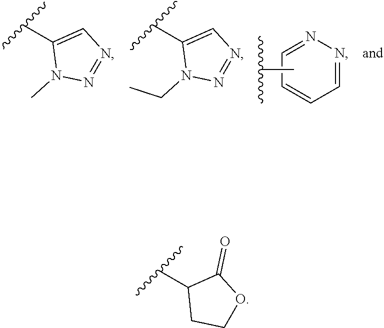

E8 A compound of embodiment E7, or a pharmaceutically acceptable salt thereof, wherein R¹ is selected from the group consisting of:

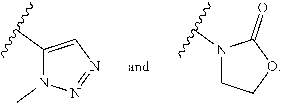

E9 A compound of embodiment E8, or a pharmaceutically acceptable salt thereof, wherein R¹ is:

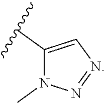 [SMILES: *c1cnnn1C]

E10 A compound of embodiment E8, or a pharmaceutically acceptable saft thereof, wherein R¹ is:

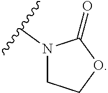 [SMILES: *N1CCOC1=O]

E11 A compound of any one of embodiments E1 to E10, or a pharmaceutically acceptable salt thereof, wherein R² and R³ are each independently selected from the group consisting of H, C₁-C₃ alkyl, C₃-C₅ cycloalkyl, C₂-C₃ alkoxyalkyl, and C₁-C₃ fluoroalkyl, or R² and R³ are taken together with the carbon atom to which they are attached to form a 3-6 membered cycloalkyl, or one of R² and R³ are taken together with the carbon atom to which they are attached to, and together with X to form a 5-6 membered ring that is fused to X.
E12 A compound of embodiment E11, or a pharmaceutically acceptable salt thereof, wherein R² and R³ are each independently selected from the group consisting of H, methyl, ethyl, isopropyl, cyclopropyl, tert-butyl, —CF₃, —CH₂—CF₃, and —CH₂O—CH₃, or R² and R³ are taken together with the carbon atom to which they are attached to form a 3-5 membered cycloalkyl, or one of R² and R³ are taken together with the carbon atom to which they are attached to, and together with X to form a 5-6 membered ring that is fused to X.
E13 A compound of embodiment E12, or a pharmaceutically acceptable salt thereof, wherein R² and R³ are each independently selected from the group consisting of H, methyl, ethyl, isopropyl, cyclopropyl, tert-butyl. —CF₃. —CH₂—CF₃, and —CH₂—O—CH₃, or R and R³ are taken together with the carbon atom to which they are attached to form a 3-5 membered cycloalkyl.
E14 A compound of any one of embodiments E1 to E13, or a pharmaceutically acceptable salt thereof, wherein R⁴ and R⁵ are each independently H or methyl.
E15 A compound of any one of embodiments E1-E14, or a stereoisomer thereof, or a pharmaceutically acceptable salt of the compound or the stereoisomer of the compound thereof, wherein the compound is selected from the group consisting of:

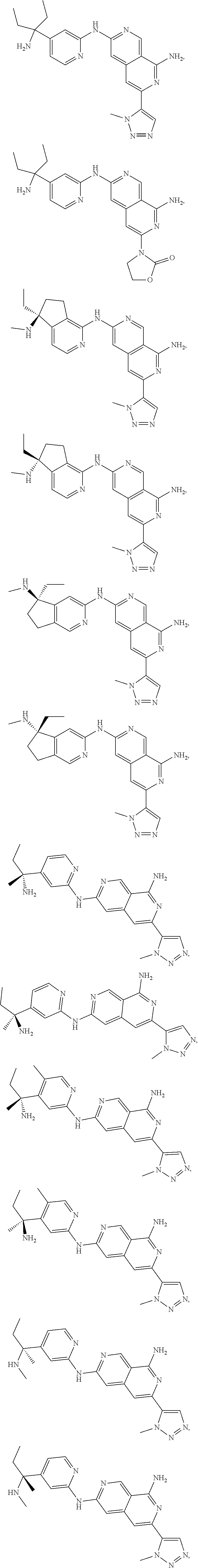

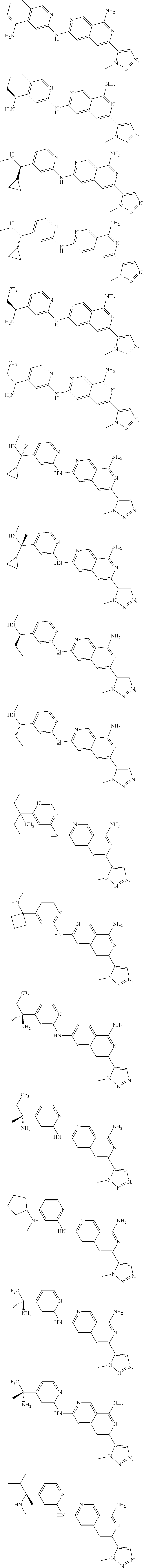

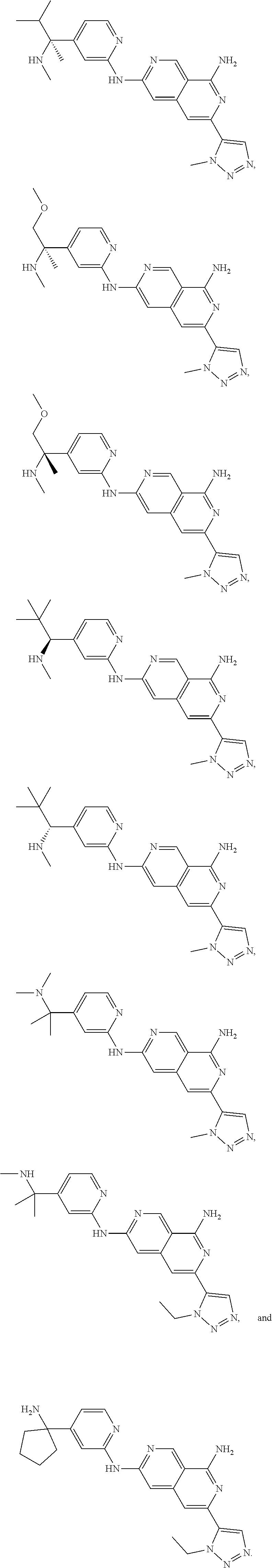

E16 A compound that is N^{B}-(4-(3-aminopentan-3-yl)pyridin-2-yl)-3-(1-methyl-1H-1,2,3-triazol-5-yl)-2,7-naphthyridine-1,6-diamine, having the structure:

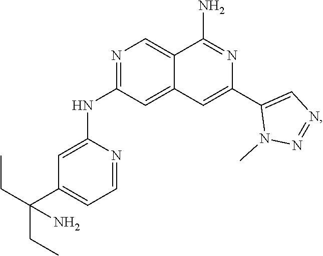 [SMILES: CCC(N)(CC)c1ccnc(Nc2cc3cc(-c4cnnn4C)nc(N)c3cn2)c1]

or a pharmaceutically acceptable salt thereof.
E17 A compound that is N⁶-(4-(3-aminopentan-3-yl)pyridin-2-yl)-3-(1-methyl-1H-1,2,3-triazol-5-yl)-2,7-naphthyridine-1,6-diamine, having the structure:

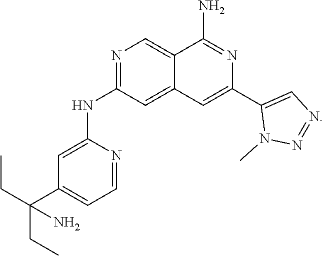 [SMILES: CCC(N)(CC)c1ccnc(Nc2cc3cc(-c4cnnn4C)nc(N)c3cn2)c1]

E18 A pharmaceutically acceptable salt of a compound, wherein the compound is N⁶-(4-(3-aminopentan-3-yl)pyridin-2-yl)-3-(1-methyl-1H-1,2,3-triazol-5-yl)-2,7-naphthyridine-1,6-diamine, having the structure:

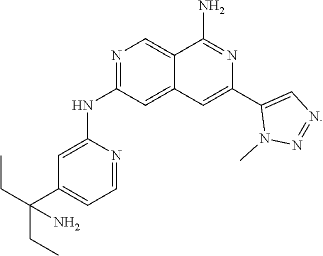 [SMILES: CCC(N)(CC)c1ccnc(Nc2cc3cc(-c4cnnn4C)nc(N)c3cn2)c1]

E19 A compound that is 3-(1-amino-6-((4-(3-aminopentan-3-yl)pyridin-2-yl)amino)-2,7-naphthyridin-3-yl)oxazolidin-2-one, having the structure:

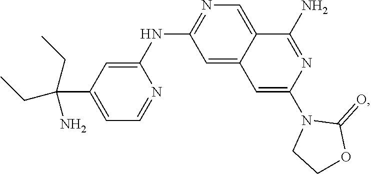 [SMILES: CCC(N)(CC)c1ccnc(Nc2cc3cc(N4CCOC4=O)nc(N)c3cn2)c1]

or a pharmaceutically acceptable salt thereof.
E20 A compound that is 3-(1-amino-6-((4-(3-aminopentan-3-yl)pyridin-2-yl)amino)-2,7-naphthyridin-3-yl)oxazolidin-2-one, having the structure:

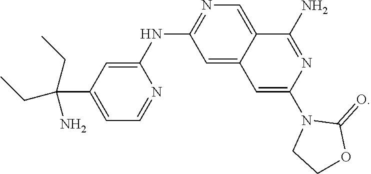 [SMILES: CCC(N)(CC)c1ccnc(Nc2cc3cc(N4CCOC4=O)nc(N)c3cn2)c1]

E21 A pharmaceutically acceptable salt of a compound, wherein the compound is 3-(1-amino-6-((4-(3-aminopentan-3-yl)pyridin-2-yl)amino)-2,7-naphthyridin-3-yl)oxazolidin-2-one, having the structure:

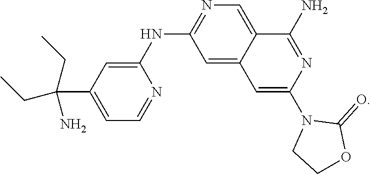 [SMILES: CCC(N)(CC)c1ccnc(Nc2cc3cc(N4CCOC4=O)nc(N)c3cn2)c1]

E22 A pharmaceutical composition comprising a compound of any one of embodiments E1
E21, or a pharmaceutically acceptable salt thereof, and at least one pharmaceutically acceptable excipient.
E23 A method for treating cancer, comprising administering to a subject in need thereof a therapeutically effective amount of a compound of any one of embodiments E1 to E21, or a pharmaceutically acceptable salt thereof.
E24 A method for treating cancer, comprising administering to a subject in need thereof a therapeutically effective amount of a compound of any one of embodiments E1 to E21, or a pharmaceutically acceptable salt thereof as a single agent.
E25 A method for treating cancer, comprising administering to a subject in need thereof a therapeutically effective amount of a compound of any one of embodiments E1 to E21, or a pharmaceutically acceptable salt thereof, and further comprising administering a therapeutically effective amount of an additional anticancer therapeutic agent.
E26 A method for treating cancer of any one embodiments E23 to E25, wherein the cancer is breast cancer, colon cancer, colorectal cancer, head and neck squamous cell carcinoma, non-small cell lung cancer (NSCLC), gastric cancer, pancreatic cancer, prostate cancer, or thyroid cancer.
E27 A method for treating cancer of embodiment E26, wherein the cancer is breast cancer, non-small cell lung cancer (NSCLC), colorectal cancer, or pancreatic cancer.
E28 A compound of any one of embodiments E1 to E21, or a pharmaceutically acceptable salt thereof, for use as a medicament.
E29 A compound of any one of embodiments E1 to E21, or a pharmaceutically acceptable salt thereof, for use in the treatment of cancer.
E30 A compound for use in the treatment of cancer according to embodiment E29, wherein the cancer is breast cancer, colon cancer, colorectal cancer, head and neck squamous cell carcinoma, non-small cell lung cancer (NSCLC), gastric cancer, pancreatic cancer, prostate cancer, or thyroid cancer.
E31 A compound for use in the treatment of cancer according to embodiment E30, wherein the cancer is breast cancer, non-small cell lung cancer (NSCLC), colorectal cancer, or pancreatic cancer.
E32 Use of a compound of any one of embodiments E1 to E21, or a pharmaceutically acceptable salt thereof, for the manufacture of a medicament for the treatment of cancer.
E33 Use of a compound, or a pharmaceutically acceptable salt thereof, for the manufacture of a medicament for the treatment of cancer according to embodiment E32, wherein the cancer is breast cancer, colon cancer, head and neck squamous cell carcinoma, non-small cell lung cancer (NSCLC), gastric cancer, pancreatic cancer, prostate cancer, or thyroid cancer.
E34 Use of a compound, or a pharmaceutically acceptable salt thereof, for the manufacture of a medicament for the treatment of cancer according to embodiment E33, wherein the cancer is breast cancer, non-small cell lung cancer (NSCLC), or pancreatic cancer.
E35 A method for the treatment of a disorder mediated by inhibition of microtubule-associated serine/threonine kinase-like (MASTL) receptor in a subject, comprising administering to the subject in need thereof a compound of any one of embodiments E1
E21, or a pharmaceutically acceptable salt thereof, in an amount that is effective for treating the disorder.
E36 A pharmaceutical combination comprising a compound of any one of embodiments E1 to E21, or a pharmaceutically acceptable salt thereof, and at least one additional therapeutic agent or a pharmaceutically acceptable salt thereof.
E37 A pharmaceutical composition comprising the pharmaceutical combination of embodiment E36 and at least one excipient.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or a pharmaceutically acceptable salt thereof.

Furthermore, each of the embodiments described herein envisions within its scope pharmaceutically acceptable salts of the compounds, stereoisomers of the compounds, and pharmaceutically acceptable salts of the stereoisomers described herein.

### Definitions

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

“Compounds of the invention” include compounds of Formula (I) and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” refers to fluoro, chloro, bromo and iodo (F, Cl, Br, I).

“Cyano” refers to a substituent having a carbon atom joined to a nitrogen atom by a triple bond, i.e., —C≡N (also depicted herein as “—CN”).

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 6 carbon atoms (“C₁-C₆ alkyl”), or 1 to 2 carbon atoms (“C₁-C₂ alkyl”). Examples include, but are not limited to, methyl, ethyl, n-propyl, isopropyl, and the like.

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain 1 to 6 carbon atoms (“C₁-C₆ alkoxy”). Alkoxy groups include, but are not limited to, methoxy, ethoxy, n-propoxy, and the like.

“Alkoxyalkyl” refers to an alkyl group, as defined herein, that is substituted by an alkoxy group, as defined herein. Examples include, but are not limited to, CH₃OCH₂— and CH₃CH₂OCH₂—.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄cycloalkyl”). Examples include, but are not limited to, cyclopropyl, cyclobutyl, cyclopentyl, cyclohexyl, cycloheptyl, and the like. Cycloalkyl groups may be optionally substituted, unsubstituted or substituted, as further defined herein.

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include monocyclic or polycyclic such as bicyclic rings. Heterocycloalkyl rings also include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system.

Heterocycloalkyl rings may contain 1 to 4 heteroatoms selected from N, O, and S(O), as ring members, or 1 to 3 ring heteroatoms, or 1 to 2 ring heteroatoms, provided that such heterocycloalkyl rings do not contain two contiguous oxygen or sulfur atoms.

Heterocycloalkyl rings may be optionally substituted, unsubstituted or substituted, as further defined herein. Such substituents may be present on the heterocyclic ring attached to the base molecule, or on a monocyclic, bicyclic, tricyclic, spirocyclic, bridged or fused ring attached thereto.

Heterocycloalkyl rings may include, but are not limited to, 4-10 membered heterocyclyl groups, for example 5-8 or 5-6 membered heterocycloalkyl groups, in accordance with the definition herein. Examples of heterocycloalkyl ring group of the present invention may include, but are not limited to aziridinyl, azetidinyl, pyrrolidinyl, piperidinyl, piperazinyl, morpholinyl, thiomorpholinyl, azepanyl, oxazepanyl, thiazepanyl, a radical of hexahydro-1H-pyrrolizine ring, a radical of 8-oxa-3-azabicyclo[3.2.1]octane ring, a radical of 3-azabicyclo[3.2.1]octane ring, a radical of 6-azabicyclo[3.2.1]octane ring, or a radical of 3-azabicyclo[3.2.0]heptane ring.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, quinolinyl, isoquinolinyl, purinyl, triazinyl, naphthyridinyl, cinnolinyl, quinazolinyl, quinoxalinyl, carbazolyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, purinyl, indolinonyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, azaquinazolinyl, phthalazinyl, (pyrido[3,2-d]pyrimidinyl, (pyrido[4,3-d]pyrimidinyl, (pyrido[3,4-d]pyrimidinyl, (pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

A wavy line “ [symbol] ” used in a chemical structure in the present disclosure refers to the point of the attachment of a substituent.

The term “pharmaceutically acceptable” means the substance (e.g., the compounds described herein) and any salt thereof, or composition containing the substance or salt of the invention is suitable for administration to a subject or patient.

The symbol “or 1” in a chemical structure means a chiral center that has been resolved into two separate enantiomers, but the specific enantiomer has not been confirmed.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula (I) may also include other salts of such compounds which are not necessarily pharmaceutically acceptable salts, which may be useful as intermediates for one or more of the following: 1) preparing compounds of Formula (I); 2) purifying compounds of Formula (I); 3) separating enantiomers of compounds of Formula (I); or 4) separating diastereomers of compounds of Formula (I).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphthalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula (I) may also include other solvates of such compounds which are not necessarily pharmaceutically acceptable solvates, which may be useful as intermediates for one or more of the following: 1) preparing compounds of Formula (I); 2) purifying compounds of Formula (I): 3) separating enantiomers of compounds of Formula (I); or 4) separating diastereomers of compounds of Formula (I).

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻Na⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University. Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes tritium, i.e., ³H, and carbon-14, i.e., ¹⁴C, are particularly useful for this purpose in view of their ease of incorporation and ready means of detection.

Substitution with deuterium, i.e., ²H, may afford certain therapeutic advantages resulting from greater metabolic stability.

Substitution with positron emitting isotopes, such as ¹¹C, ¹⁸F, ¹⁵O and ¹³N, may be useful in Positron Emission Topography (PET) studies for examining substrate receptor occupancy.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some Specific Examples of Prodrugs in Accordance with the Invention Include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to:
(i) where the compound of the invention contains an alkyl group, a hydroxyalkyl derivative thereof (—CH>—COH):
(ii) where the compound of the invention contains an alkoxy group, a hydroxy derivative thereof (—OR→—OH);
(iii) where the compound of the invention contains a tertiary amino group, a secondary amino derivative thereof (—NRR→—NHR or —NHR);
(iv) where the compound of the invention contains a secondary amino group, a primary derivative thereof (—NHR→—NH₂);
(v) where the compound of the invention contains a phenyl moiety, a phenol derivative thereof (—Ph→—PhOH);
(vi) where the compound of the invention contains an amide group, a carboxylic acid derivative thereof (—CONH₂→COOH); and
(vii) where the compound contains a hydroxy or carboxylic acid group, the compound may be metabolized by conjugation, for example with glucuronic acid to form a glucuronide.

Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

The term “excipient” is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gellan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Uberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250, 500 or 1000 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example. “Amorphous Solid Dispersions Theory and Practice” by Navnit Shah et al.

### Administration and Dosing

The term “treating”, “treat” or “treatment” as used herein embraces both preventative. i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, a compound of the invention is administered in an amount effective to treat a condition as described herein. The compounds of the invention may be administered as compound per se, or alternatively, as a pharmaceutically acceptable saft. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds of the invention may inhibit the activities of MASTL, and may be useful in the treatment, prevention, suppression, and amelioration of diseases such as cancers, disorders and conditions mediated by MASTL.

Cancers to be treated include squamous cell carcinoma, basal cell carcinomas, myeloma, small-cell lung cancer, non-small cell lung cancer (NSCLC), glioma, Hodgkin's lymphoma, non-Hodgkin's lymphoma, acute myeloid leukemia (AML), multiple myeloma, gastric cancer, renal cancer, ovarian cancer, liver cancer, lymphoblastic leukemia, lymphocytic leukemia, colon cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, uterine cancer, bladder cancer, including non-muscular invasive bladder cancer, hepatoma, breast cancer, and head and neck cancer.

Preferably, the compounds of the present invention may be useful for the treatment of breast cancer, colon cancer, colorectal cancer, head and neck cancer, non-small cell lung cancer (NSCLC), gastric cancer, pancreatic cancer, prostate cancer, or thyroid cancer. (See Fatima et al., Cancer Medicine. 2020; 9:6322-6329).

More preferably, the compounds of the present invention may be useful for the treatment of breast cancer, non-small cell lung cancer (NSCLC), colorectal cancer, or pancreatic cancer.

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic anticancer agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration.” “co-administration,” “simultaneous administration.” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

Classes of additional chemotherapeutic agents, which can be administered in combination with a compound of this invention, include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists; IL-2 receptor agonist (recombinant cytokines or agonists for cytokine receptors); and anti-sense oligonucleotides or oligonucleotides derivatives that inhibit expression of genes implicated in abnormal cell proliferation or tumor growth.

Other additional chemotherapy agents include not only taxanes or platinum agents but also HER2 targeted agents, e.g., trastuzumab.

In another embodiment, such additional anti-cancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, spindle poison plant alkaloids, KRAS inhibitors, MCT4 inhibitors, MAT2a inhibitors, alk/c-Met/ROS inhibitors (including crizotinib or lorlatinib), mTOR inhibitors (including temsirolimus or gedatolisib), src/abl inhibitors (including bosutinib), cyclin-dependent kinase (CDK) inhibitors (including palbociclib, PF-06873600), erb inhibitors (including dacomitinib), PARP inhibitors (including talazoparib), SMO inhibitors (including glasdegib), EGFR T790 M inhibitors, PRMT5 inhibitors, TGFβR1 inhibitors, growth factor inhibitors, cell cycle inhibitors, biological response modifiers, enzyme inhibitors, and cytotoxics.

In another embodiment, such additional anti-cancer therapeutic agents include compounds derived from an anti-angiogenesis agent, including for example tyrosine kinase/vascular endothelial growth factor (VEGF) receptor (VEGFR) inhibitors (including sunitinib, axitinib, sorafenib, and tivozanib), TIE-2 inhibitors, PDGFR inhibitors, angiopoietin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™), axitinib (Inlyta™), SU 14813 (Pfizer), and AG 13958 (Pfizer). Additional anti-angiogenesis agents include vatalanib (CGP 79787), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Yet further anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voftaren™), indomethacin (Indocin™), sulindac (Clinoril™), tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon). Yet further anti-angiogenesis agents include acitretin (Neotigason™), plitidepsin (Aplidine™), cilengitide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment, such additional anti-cancer therapeutic agents include compounds derived from hormonal agents and antagonists. Examples include where anti-hormonal agents act to regulate or inhibit hormone action on tumors such as anti-estrogens and selective estrogen receptor modulators (SERMs), and a selective estrogen receptor degrader (SERD) including tamoxifen, raloxifene, droloxifene, 4-hydroxytamoxifen, trioxifene, keoxifene. LY117018, onapristone, toremifene (Fareston), and fulvestrant. Examples also include aromatase inhibitors that inhibit the enzyme aromatase, which regulates estrogen production in the adrenal glands, and include compounds like 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin.

In another embodiment, such additional anti-cancer therapeutic agents include compounds derived from signal transduction inhibitors, such as inhibitors of protein tyrosine kinases and/or serine/threonine kinases: a signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors. EGF inhibitor, ErbB-1 (EGFR), ErbB-2, pan erb, IGF1R inhibitors, MEK (including binimetinib (Mektovi™)), c-Kit inhibitors, FLT-3 inhibitors, K-Ras inhibitors, PI3 kinase inhibitors, JAK inhibitors, STAT inhibitors, Raf kinase inhibitors, BRAF (including encorafenib (Braftovi™)), Akt inhibitors, mTOR inhibitor. P70S6 kinase inhibitors, inhibitors of the WNT pathway and multi-targeted kinase inhibitors.

In another embodiment, such additional anti-cancer therapeutic agents include docetaxel, paclitaxel, paclitaxel protein-bound particles, cisplatin, carboplatin, oxaliplatin, capecitabine, gemcitabine or vinorelbine.

In another embodiment, such additional anti-cancer therapeutic agents include compounds derived from an epigenetic modulator, where examples include an inhibitor of EZH2 (including PF-06821497), SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In another embodiment, such additional anti-cancer therapeutic agents include compounds that are immuno-oncology agents, including immunomodulatory agents.

In another embodiment, combinations with pattern recognition receptors (PRRs) are contemplated. PRRs are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-1-like receptors (RLRs), nucleotide-binding oligomerization domain (NOD)-like receptors (NLRs), C-type lectin receptors (CLRs), and Stimulator of Interferon Genes (STING) protein.

The STING protein functions as both a cytosolic DNA sensor and an adaptor protein in Type 1 interferon signaling. The terms “STING” and “stimulator of interferon genes” refer to any form of the STING protein, as well as variants, isoforms, and species homologs that retain at least a part of the activity of STING. Unless indicated differently, such as by specific reference to human STING, STING includes all mammalian species of native sequence STING, e.g. human, monkey, and mouse STING is also known as—TMEM173.

“STING agonist” as used herein means, any molecule, which upon binding to STING, (1) stimulates or activates STING, (2) enhances, increases, promotes, induces, or prolongs an activity, function, or presence of STING, or (3) enhances, increases, promotes, or induces the expression of STING. STING agonists useful in the any of the treatment method, medicaments and uses of the present invention include, for example, nucleic acid ligands which bind STING.

Examples of STING agonists that are useful in the treatment methods, medicaments, and uses of the present invention include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as WO2019027858, WO20180093964. WO2017175156, WO2017175147.

Therapeutic antibodies may have specificity against a variety of different antigens. For example, therapeutic antibodies may be directed to a tumor associated-antigen, such that binding of the antibody to the antigen promotes death of the cell expressing the antigen. In other example, therapeutic antibodies may be directed to an antigen on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

In another embodiment, such additional anti-cancer therapeutic agents include antibodies that would be blocking or inhibitory at the target: CTLA-4 (including ipilimumab or tremelimumab), PD-1 or PD-L1 (including atezolizumab, avelumab, cemiplimab, durvalumab, nivolumab, sasanlimab, or pembrolizumab), LAG-3, TIM-3, or TIGIT.

In another embodiment, such additional anti-cancer therapeutic agents include antibodies that are agonists of 4-1BB, OX40, GITR, ICOS, or CD40.

In another embodiment the anti-cancer therapy may be a CAR-T-cell therapy.

Examples of a therapeutic antibody include: an anti-OX40 antibody, an anti-4-1BB antibody, an anti-HER2 antibody (including an anti-HER2 antibody-drug conjugate (ADC)), a bispecific anti-CD47/anti-PD-L1 antibody, and a bispecific anti-P-cadherin/anti-CD3 antibody. Examples of cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof. Exemplary immunomodulating agents that may be incorporated in an ADC include gancyclovir, etanercept, tacrolimus, sirolimus, voclosporin, cyclosporine, rapamycin, cyclophosphamide, azathioprine, mycophenolate mofetil, methotrexate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-15, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta, and -.gamma), the stem cell growth factor designated “S 1 factor,” erythropoietin and thrombopoietin, or a combination thereof.

Additional examples of therapeutic antibodies may include the following antigens where exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. see U.S. Pat. No. 9,969,809]; BTN1A1 (e.g. see WO2018222689); CA-125 (e.g. abagovomab); Carboanhydrase IX; CCR2; CCR4 (e.g. mogamulizumab); CCR5 (e.g. leronlimab); CCR8; CD3 [e.g. blinatumomab (CD3/CD19 bispecific), CD3/P-cadherin bispecific, CD3/BCMA bispecific] CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD123; CD276/B7-H3 (e.g. omburtamab); CDH17; CEA; ClhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); GD2 (e.g. dinutuximab, 3F8); GD3; GITR; GloboH; GM1; GM2; HER2/neu [e.g. margetuximab, pertuzumab, trastuzumab; ado-trastuzumab emtansine, trastuzumab duocarmazine, [see U.S. Pat. No. 8,828,401]; HER3; HER4; ICOS; IL-10; ITG-AvB6; LAG-3 (e.g. relatlimab); Lewis-Y; LG; Ly-6; M-CSF [see U.S. Pat. No. 7,326,414]; MCSP; mesothelin; MUC1; MUC2; MUC3; MUC4; MUCSAC; MUC5B; MUC7; MUC16; Notch1; Notch3; Nectin-4 (e.g. enfortumab vedotin); OX40 [see U.S. Pat. No. 7,960,515]; P-Cadherein [see WO2016/001810]; PCDHB2; PDGFRA (e.g. olaratumab); Plasma Cell Antigen; PolySA; PSCA; PSMA; PTK7 [see U.S. Pat. No. 9,409,995]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g. sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7,10-tetraazacyclododecane-N,N′,N″,N′″-tetraacetic acid (DOTA), 1,4,7-triazacyclononane-1,4,7-tetracetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

The invention also concerns the use of radiation in combination with any anti-cancer therapeutic agent administered herein. More specifically, compounds of the invention can be administered in combination with additional therapies, such as radiation therapy and/or chemotherapy.

These agents and compounds of the invention may be combined with pharmaceutically acceptable vehicles such as saline, Ringers solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-tert-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### GENERAL EXPERIMENTAL DETAILS

¹H and ¹⁹F Nuclear Magnetic Resonance (NMR) spectra were recorded on Bruker XWIN-NMR (400 or 600 MHz) spectrometer. ¹H and ¹⁹F resonances are reported in parts per million (ppm) downfield from tetramethylsilane. ¹H NMR data are reported as multiplicity (e.g. s, singlet; d, doublet; t, triplet; q, quartet; quint, quintuplet; dd, doublet of doublets; dt, doublet of triplets; br s, broad singlet). For spectra obtained in CDCl₃, DMSO-d₆, and CD₃OD, the residual protons (7.27, 2.50, and 3.31 ppm, respectively) were used as the internal reference. All observed coupling constants, J, are reported in Hertz (Hz). Exchangeable protons are not always observed.

Optical rotations were determined on a Jasco P-2000 or a Rudolph Autopol IV polarimeter. All final compounds were purified to a 95% purity, unless otherwise specified. When absolute stereochemistry is known, (R,S) labels are used. When absolute stereochemistry is not known, the software-generated names are modified to include (+)- and (−)-prefixes according to the optical rotations, and (R*/S) labels are used to show relative configuration.

Mass spectra. MS (m/z), were recorded using either electrospray ionization (ESI) or atmospheric pressure chemical ionization (APCI). Where relevant and unless otherwise stated, the m/z data provided are for isotopes ¹⁹F, ³⁵Cl, ⁷⁹Br and ¹²⁷I.

The nomenclature is written as described by IUPAC (International Union of Pure and Applied Chemistry generated within Perkin Elmers Chemdraw 18.0.0.231. The naming convention provided with Perkin Elmers Chemdraw 18.0.0.231 is well known by those skilled in the art and it is believed that the naming convention provided with Perkin Elmers Chemdraw 18.0.0.231 generally comports with the IUPAC (International Union for Pure and Applied Chemistry) recommendations on Nomenclature of Organic Chemistry and the CAS Index rules.

### ABBREVIATIONS

AcOH is acetic acid;
aq is aqueous;
BINAP is 1,1′-binaphthalene-2,2′-diyl)bis(diphenylphosphine;
Bn is benzyl;
Boc is ted-butoxycarbonyl;
Boc₂O is di-tert-butyl dicarbonate;
br is broad;
tBu is tert-butyl;
° C. is degrees celcius;
CDCl₃ is deutero-chloroform;
δ is chemical shift;
d is doublet;
dd is doublet of doublets;
ddd is doublet of doublet of doublets;
dt is doublet of triplets;
DCM is dichloromethane; methylene chloride;
DIPEA is N-ethyldiisopropylamine, also known as N,N-diisopropylethylamine;
DMA is N,N-dimethylacetamide;
DMF is N,N-dimethylformamide;
DMSO is dimethyl sulfoxide;
DMSO-d₆ is deuterodimethylsulfoxide:
Et₂O is diethyl ether;
EtOAc is ethyl acetate;
EtOH is ethanol;
Et₃N is triethylamine;
g is gram;
HPLC is high pressure liquid chromatography;
hr(s) is hour(s);
L is liter;
LCMS is liquid chromatography mass spectrometry;
LHMDS is Lithium bis(trimethylsilyl)amide;
m is multiplet;
M is molar;
MeOD_d₄ is deuterated methanol;
MeOH is methanol;
mg is milligram;
MHz is mega Hertz;
min(s) is minute(s);
mL is milliliter;
mmol is millimole;
mol is mole;
MS (m/z) is mass spectrum peak;
NMR is nuclear magnetic resonance;
PE is petroleum ethers;
pH is power of hydrogen;
PMB is para-methoxybenzyl;
ppm is parts per million;
q is quartet;
rt is room temperature;
RT is retention time;
s is singlet;
SFC is supercritical fluid chromatography;
t is triplet;
TBAF is tert-butyl ammonium fluoride;
TFA is trifluoroacetic acid;
THF is tetrahydrofuran;
TLC is thin layer chromatography;
μL is microliter, and
μmol is micromole.

The schemes described below are intended to provide a general description of the methodology employed in the preparation of the compounds of the present invention. Some of the compounds of the present invention contain a single chiral center. In the following schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### INTERMEDIATES

### Synthesis of 6-chloro-3-(1-methyl-1H-1,2,3-triazol-5-yl)-2,7-naphthyridin-1-amine (A-3)

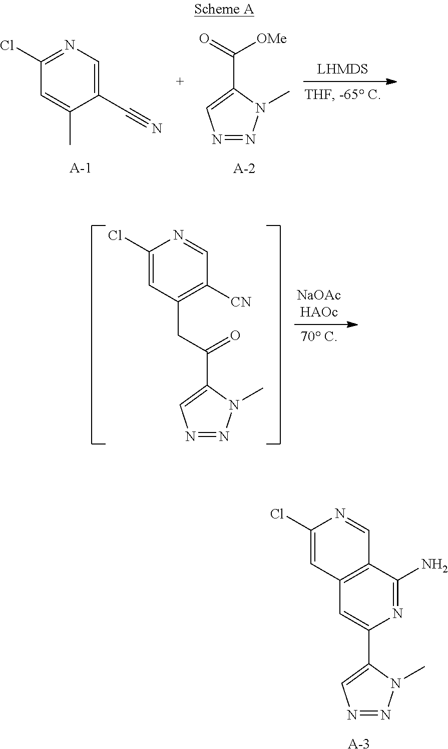

To a mixture of A-1 (27.86 g) in dry THF (300 mL) was added dropwise LHMDS (1 M, 638.84 mL) over 90 min at −65° C. under N₂. The mixture was stirred at −65° C. for 30 min, then a solution of A-2 (25.77 g, 182.62 mmol) in dry THF (250 ml) was added dropwise over 30 min at −65° C. The reaction mixture was gradually warmed to 20° C. and stirred at this temperature for 1 h. TLC showed the starting material was consumed and the desired ketone intermediate was detected. To the reaction mixture was added NH₄OAc (141.46 g, 1.84 mol) in one portion at 20° C., followed by dropwise addition of AcOH (1.10 kg, 18.36 mol, 1.05 L) over 3 h at 0° C. The resulting yellow mixture was heated at 70° C. for 3 hrs. TLC showed the ketone was consumed and A-3 was detected. The mixture was concentrated to remove THF and most of the AcOH. The residue was diluted with EtOAc (300 mL) and carefully neutralized to pH-8 with aq. K₂CO₃ and stirring. The suspension was stirred at 20° C. for 1 h then filtered. The filter cake was washed with DCM (100 mL×3), H₂O (100 mL×3) and PE (100 mL×3). The filter cake was dried in a vacuum oven at 50° C. to give A-3 (163.4 g, 65%) as a brown solid. LCMS 261.0 [M+1]; ¹H NMR (400 MHz, DMSO-d6) δ=9.41 (s, 1H), 8.16 (s, 1H), 7.76 (s, 1H), 7.74 (br s, 2H), 7.34 (s, 1H), 4.37 (s, 3H).

### Synthesis of N¹,N¹-bis(4-methoxybenzyl)-3-(1-methyl-1H-1,2,3-triazol-5-yl)-2,7-naphthyridine-1,6-diamine (B-3)

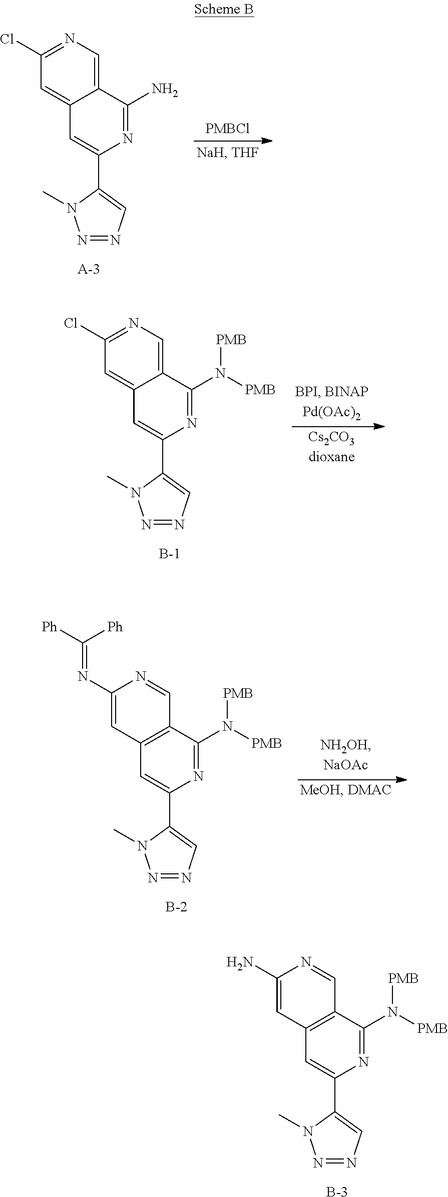

Step 1—Synthesis of 6-chloro-N,N-bis(4-methoxybenzyl)-3-(1-methyl-1H-1,2,3-triazol-5-yl)-2,7-naphthyridin-1-amine (B-1) A solution of A-3 (25.0 g, 96 mmol) in DMA (959 mL) was cooled in an ice bath then NaH (12.7 g, 318 mmol) was added in 3 portions. After 15 min, 4-methoxybenzylchloride (42.8 g, 273 mmol) was added dropwise and the reaction was stirred in the ice batch then slowly warmed to 12° C. for another 12 h. The mixture was cooled to 0° C., quenched by addition of 900 mL of H₂O. After being stirred at room temperature for 20 min, the solid was removed by filtration. The filter cake was washed with EtOAc then dried under vacuum to give B-1 (27.2 g, 57%) as a yellow solid. LCMS 501.2 [M+1]; ¹H NMR (400 MHz, CHLOROFORM-d) δ=9.21 (s, 1H), 8.01 (s, 1H), 7.58 (s, 1H), 7.23 (s, 1H), 7.12 (d, J=8.6 Hz, 4H), 6.85 (d, J=8.6 Hz, 4H), 4.83 (s, 4H), 4.06 (s, 3H), 3.78 (s, 6H).

### Step 2—Synthesis of 6-((diphenylmethylene)amino)-N,N-bis(4-methoxybenzyl)-3-(1-methyl-1H-1,2,3-triazol-5-yl)-2,7-naphthyridin-1-amine (B-2)

To a solution of B-1 (27.2 g, 54.3 mmol) in 1,4-dioxane (362 mL) was added benzophenone imine (16.7 g, 92.3 mmol), Cs₂CO₃ (44.2 g, 136 mmol), BINAP (5.41 g, 8.69 mmol) and Pd(OAc)₂ (0.97 g, 4.34 mmol). The reaction mixture was purged with N₂ for 3 min then stirred at 100° C. under nitrogen for 12 h. The crude reaction was diluted with EtOAc and brine. The layers were separated, and the organic phase was dried (MgSO4), filtered and concentrated to give B-2 (43.2 g, 100%) as a brown gum. The crude product was used directly for the next step without purification.

### Step 3—Synthesis of N¹,N¹-bis(4-methoxybenzyl)-3-(1-methyl-1H-1,2,3-triazol-5-yl)-2,7-naphthyridine-1,6-diamine (B-3)

To a solution of B-2 (43.2 g, 66.9 mmol) in DMA (160 mL) was added MeOH (530 mL) and NH₂OH (8.84 g, 50% in water, 124 mmol) followed by NaOAc (11.0 g, 134 mmol) at 13° C. The mixture was stirred at 13° C. for 16 h. The crude reaction was concentrated then diluted with EtOAc and brine. The layers were separated, and the organic phase was dried (MgSO4), filtered and concentrated. The crude residue was purified by column chromatography (ISCO, 80 g SiO₂, 0-100% EtOAc/PE) to give B-3 (16.6 g, 51%) as a yellow solid. LCMS 482.4 [M+1]; ¹H NMR (400 MHz, DMSO-d6) δ=8.99 (s, 1H), 8.16 (s, 1H), 7.39-7.34 (m, 11H), 7.20 (d, J=8.6 Hz, 4H), 6.89 (d, J=8.7 Hz, 4H), 6.50 (d, J=6.7 Hz, 3H), 4.69 (s, 4H), 4.07-4.00 (m, 3H), 3.72 (s, 6H).

### Synthesis of 3-1-ethyl-1H-1,2,3-triazol-5-yl)-N¹,N¹-bis(4-methoxybenzyl)-2,7-naphthyridine-1,6-diamine (C-1)

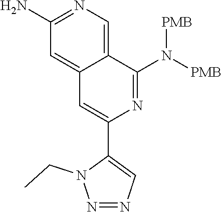

C-1 was made in a similar fashion to B-3, using 6-chloro-3-(1-ethyl-1H-1,2,3-triazol-5-yl)-2,7-naphthyridin-1-amine in place of 6-chloro-3-(1-methyl-1H-1,2,3-triazol-5-yl)-2,7-naphthyridin-1-amine (A-3) and using methyl 1-ethyl-1H-1,2,3-triazole-5-carboxylate in place of methyl 1-methyl-1H-1,2,3-triazole-5-carboxylate (A-2). LCMS 496.2 [M+1]; ¹H NMR (400 MHz, DMSO-d6) δ=9.02 (s, 1H), 8.14 (s, 1H), 7.41-7.34 (m, 1H), 7.18 (d, J=8.5 Hz, 4H), 6.89 (d, J=8.8 Hz, 4H), 6.64-6.42 (m, 2H), 4.68 (s, 4H), 4.51 (q, J=7.1 Hz, 2H), 3.72 (s, 6H), 1.11 (t, J=7.2 Hz, 3H).

### Synthesis of 3-(6-amino-1-(bis(4-methoxybenzyl)amino)-2,7-naphthyridin-3-yl)oxazolidin-2-one (D-3)

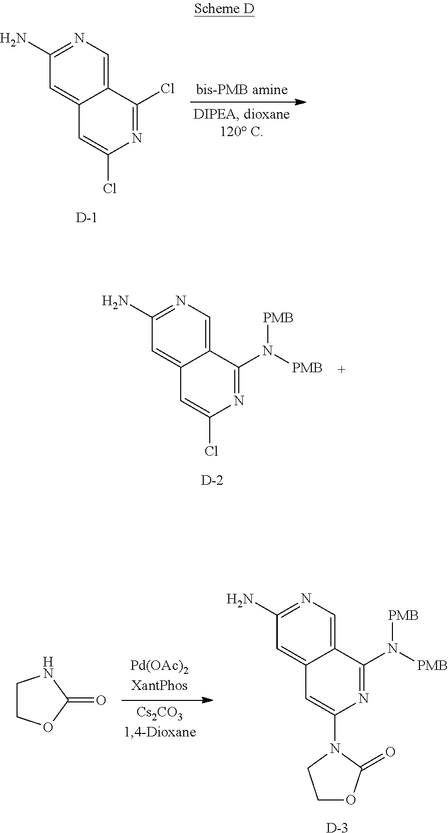

### Step 1—Synthesis of 3-chloro-N¹,N¹-bis(4-methoxybenzyl)-2,7-naphthyridine-1,6-diamine (D-2)

To a mixture of D-1 (2500 mg, 11.68 mmol) and bis(4-methoxybenzyl)amine (3310 mg, 12.8 mmol) in 1,4-dioxane (29 mL) was added DIPEA (3020 mg, 23.4 mmol) at 20° C. After addition, the mixture was stirred at 120° C. for 64 h then at 130° C. for 24 h. The crude reaction was concentrated and purified by column chromatography (40 silica gel, DCM:MeOH (10% NH₄OH) followed by Prep-HPLC to give D-2 (1.8 g, 35%) as a yellow solid. LCMS 435.3 [M+1]; ¹H NMR (400 MHz, CHLOROFORM-d) δ=9.01 (s, 1H), 7.32-7.28 (m, 4H), 6.92 (d, J=8.5 Hz, 4H), 6.82 (s, 1H), 6.46 (s, 1H), 4.76 (s, 4H), 4.68 (br s, 2H), 3.85 (s, 6H).

### Step 2—Synthesis of 3-(6-amino-1-(bis(4-methoxybenzyl)amino)-2,7-naphthyridin-3-yl)oxazolidin-2-one (D-3)

To a mixture of D-2 (1650 mg, 3.79 mmol), 2-oxazolidinone (496 mg, 5.69 mmol) and Cs₂CO₃ (2470 mg, 7.59 mmol) in 1,4-dioxane (76 mL) was added Pd₂(dba)₃ (347 mg, 0.38 mmol) and Xantphos (439 mg, 0.76 mmol). The reaction was stirred at 100° C. for 16 h under N₂. The crude reaction mixture was extracted with EtOAc (100 mL×5). The combined organic layers were washed with brine (100 mL) then dried over Na₂SO₄, filtered and concentrated. The residue was purified by column chromatography (20 g silica gel, 0-100% EtOA/PE, 0-20% MeOH/DCM) to give the crude D-3. The resulting brown solid was triturated with DCM (40 mL) and MeOH (2 mL) to give D-3 (950 mg, 47%) as a yellow solid. LCMS 486.2 [M+1]; ¹H NMR (400 MHz, CHLOROFORM-d) δ=8.97 (s, 1H), 7.59 (s, 1H), 7.18 (d, J=8.6 Hz, 4H), 6.87 (d, J=8.6 Hz, 4H), 6.54 (s, 1H), 4.72 (s, 4H), 4.59 (s, 2H), 4.42-4.34 (m, 2H), 4.12-4.05 (m, 2H), 3.83-3.78 (m, 6H).

### Synthesis of N-(3-(2-aminopyridin-4-yl)pentan-3-yl)-2-methylpropane-2-sulfinamide (E-6)

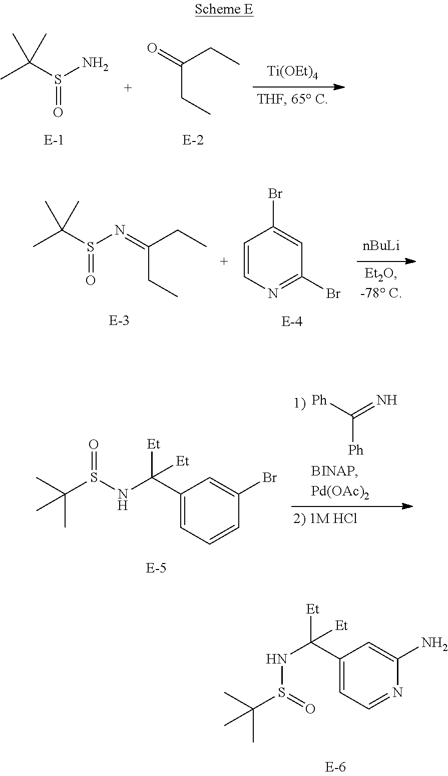

### Step 1—Synthesis of 2-methyl-N-(pentan-3-ylidene)propane-2-sulfinamide (E-3)

To a solution of E-2 (200 g, 2.32 mol) and Ti(OEt)₄ (1.06 kg, 4.64 mol) in THF (1500 mL) was added (S)-2-methylpropane-2-sulfinamide (281.43 g, 2.32 mol), and the mixture was stirred at 65° C. for 16 h. The reaction mixture was cooled to rt then poured into 2 L of brine. The mixture was filtered through a short pad of Celite with EtOAc as eluent. The organic layer was separated, washed with brine, then dried over Na₂SO₄, and concentrated. The crude product was purified by column chromatography (0-40% EtOAc/PE) to give E-3 (331 g, 15%) as a yellow oil. LCMS 190.1 [M+1]; ¹H NMR (400 MHz, CHLOROFORM-d) δ=2.72 (q, J=7.5 Hz, 2H), 2.46 (td, J=6.7, 13.5 Hz, 2H), 1.25 (s, 9H), 1.20 (br t, J=7.5 Hz, 3H), 1.11 (br t, J=7.0 Hz, 3H).

### Step 2—Synthesis of N-(3-(2-bromopyridin-4-yl)pentan-3-yl)-2-methylpropane-2-sulfinamide (E-5)

A solution of 2,4-dibromopyridine (200.21 g, 845.14 mmol) in i-Pr₂O (2400 mL) was cooled to −78° C. n-BuLi (2.5 M, 338.06 mL) was added dropwise at −78° C. The mixture was stirred at −78° C. for 15 min then E-3 (80 g, 422.57 mmol) in i-Pr₂O (800 mL) was added dropwise at −78° C. The cold bath was removed the reaction was warmed to 25° C. for 1 h. The reaction mixture was poured into sat. NH₄Cl (2000 mL) at 10° C., then filtered. The filtrate was extracted with EtOAc (1500 mL×2). The organic layer was dried over Na₂SO₄ then concentrated under vacuum. The crude product was purified by column chromatography (0-70% EtOAc/PE) to give E-5 (677 g, 50%) as a brown oil. LCMS 349.0, 347.0 [M+1]; ¹H NMR (400 MHz, CHLOROFORM-d) δ=8.35 (d, J=5.3 Hz, 1H), 7.49 (d, J=1.3 Hz, 1H), 7.29-7.25 (m, 1H), 3.62 (s, 1H), 2.19 (qd, J=7.3, 14.4 Hz, 1H), 2.11-1.94 (m, 3H), 1.30 (s, 9H), 0.77 (q, J=7.2 Hz, 6H).

### Step 3—Synthesis of N-(3-(2-aminopyridin-4-yl)pentan-3-yl)-2-methylpropane-2-sulfinamide (E-6)

A mixture of E-5 (133 g, 382.94 mmol), diphenylmethanimine (83.28 g, 459.53 mmol), BINAP (23.84 g, 38.29 mmol) and Cs₂CO₃ (374.31 g, 1.15 mol) in toluene (2000 mL) was degassed and purged with N₂ 3 times then Pd(OAc)₂ (8.60 g, 38.29 mmol) was added. The mixture was degassed and purged with N₂ 3 times then stirred at 110° C. for 18 h under a N₂ atmosphere. The reaction mixture was filtered through Celite and the filtrate was concentrated in vacuum. To a solution of crude product (217.5 g, 485.89 mmol) in THF (1100 mL)/H₂O (1100 mL) was added dropwise HCl (1 M, 971.78 mL) at 0° C. and the mixture was stirred at 25° C. for 1 hour. The mixture was extracted with MTBE (1000 mL×2). The aqueous phase was basified with 1 M aq. NaOH to pH=9 then extracted with dichloromethane (1500 mL×3). The combined organic layers were dried over Na₂SO₄, filtered then concentrated to give the crude product. The crude product was purified by column chromatography (0-10% MeOH/DCM) to give E-6 (167 g, 38% over 2 steps) as a brown solid. LCMS 284.1 [M+1]; ¹H NMR (400 MHz, CHLOROFORM-d) δ=8.04 (d, J=5.5 Hz, 1H), 6.63 (dd. J=1.7, 5.6 Hz, 1H), 6.52 (d, J=1.0 Hz, 1H), 4.43 (br s, 2H), 3.60 (s, 1H), 2.19-2.08 (m, 1H), 2.08-1.88 (m, 3H), 1.30 (s, 9H), 0.76 (td, J=7.3, 9.4 Hz, 6H).

### Synthesis of N-(1-amino-5-ethyl-6,7-dihydro-5H-cyclopenta[c]pyridin-5-yl)acetamide (F-11)

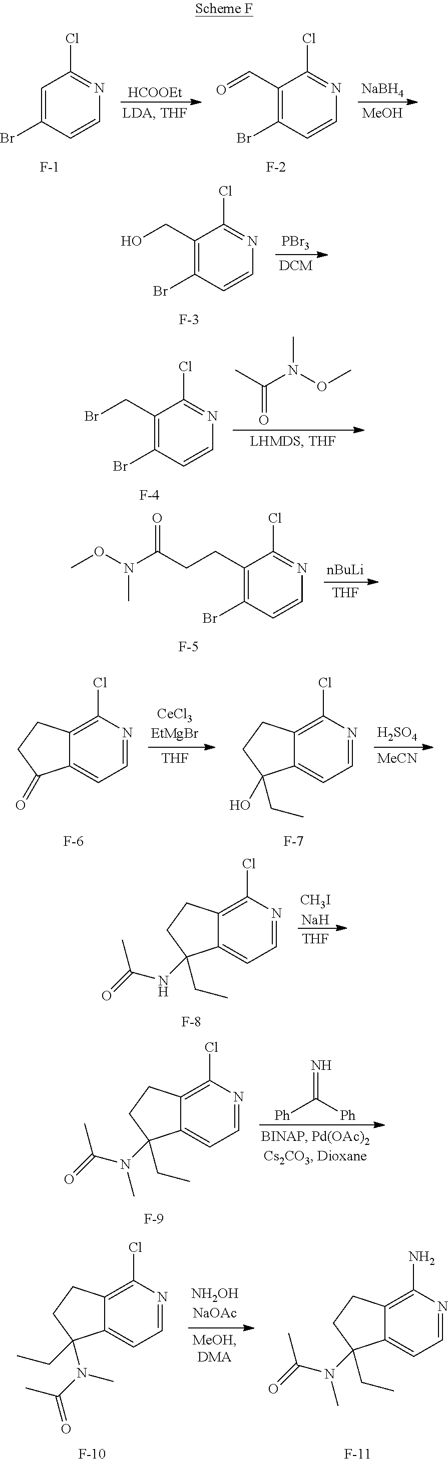

### Step 1—Synthesis of 4-bromo-2-chloronicotinaldehyde (F-2)

To a cooled (−70° C.) solution of bromo-2-chloropyridine (F-1) (73.0 g, 379 mmol) in THF (800 mL) was added LDA (42.7 g, 398 mmol) over a period of 1.5 h. The reaction was stirred for another 1 h at −70° C. then HCOOEt (42.2 g, 569 mmol) was added dropwise over a period of 30 minutes. After the addition, the mixture was allowed to stir at −70° C. for 1.5 h. The reaction was carefully quenched with saturated aq. NH₄Cl (200 mL) then diluted with EtOAc (500 mL) and brine (200 mL). The organic phase was separated and the aqueous phase was extracted with EtOAc (200 mL×2). The combined organic phases were washed with brine (200 mL), dried over anhydrous Na₂SO₄, filtered and concentrated. The crude residue was recrystallized with EtOAc/PE (500 mL, 1/20) to give F-2 (75 g, 89%) as yellow solid. ¹H NMR (400 MHz, CHLOROFORM-d) δ=10.38 (br d, J=2.9 Hz, 1H), 8.43-8.17 (m, 1H), 7.76-7.50 (m, 1H).

### Step 2—Synthesis of (4-bromo-2-chloropyridin-3-yl) methanol (F-3)

To a solution of F-2 (82.2 g, 373 mmol) in MeOH (1000 mL) was added portion-wise of NaBH₄ (10.0 g, 264 mmol) under cooling with ice-water over a period of 45 min. After the addition, the mixture was allowed to stir at the same temperature for 1 h. The reaction mixture was carefully quenched with saturated aqueous NH₄Cl (200 mL), then concentrated to remove MeOH. The mixture was diluted with EtOAc (500 mL) and water (100 mL), the organic phase was separated and the aqueous was extracted with EtOAc (100 mL×3). The combined organic phases were washed with brine (100 mL×2) then dried (Na₂SO₄), filtered and concentrated to give F-3 (76 g, 92%) as light brown solid. ¹H NMR (400 MHz, CHLOROFORM-d) δ=8.11 (d, J=5.5 Hz, 1H), 7.48 (d, J=5.4 Hz, 1H), 4.97 (br s, 2H), 2.66 (br s, 1H).

### Step 3—Synthesis of 4-bromo-3-(bromomethyl)-2-chloropyridine (F-4)

To a solution of F-3 (76.0 g, 342 mmol) in DCM (1000 mL) was added dropwise PBr₃ (139 g, 512 mmol) while the temperature was maintained between 0-5° C. over a period of 1 h. The resulting cloudy mixture was stirred at 10° C. for 1.5 h. The crude reaction mixture was adjusted to pH 8 with 1 M of aq. NaOH (keeping the inner temperature around 10-20° C.). The organic phase was separated and washed with water (100 mL×2) and brine (100 mL×2) then dried (Na₂SO₄), filtered and concentrated to give F-4 (69.6 g, 71%) as a light brown solid.

### Step 4—Synthesis of 3-(4-bromo-2-chloropyridin-3-yl)-N-methoxy-N-methylpropanamide (F-5)

To a solution of N-methoxy-N-methylacetamide (46.7 g, 453 mmol) in THF (1000 mL) under cooling with dry-ice at −70° C. was added dropwise LHMDS (75.8 g, 453 mmol). After addition, the mixture was allowed to stir at the same temperature for 30 minutes, followed by addition of F-4 (64.6 g, 226 mmol) in a solution of THF (20 mL) dropwise while maintaining the inner temperature below −65° C. The mixture was allowed to stir at −65° C. for 1 h then quenched with saturated aqueous NH₄Cl (250 mL) and diluted with EtOAc (500 mL). The organic phase was separated and the aqueous was extracted with EtOAc (100 mL×3). The combined organic phases were washed with brine (500 mL), then dried over anhydrous Na₂SO₄, filtered and concentrated. The crude product was then purified by column chromatography (330 g silica gel, 0-30% EtOAc/PE) to give F-5 (54 g, 78% crude yield) as a yellow oil. LCMS 308.9 [M+1].

### Step 5—Synthesis of 1-chloro-6,7-dihydro-5H-cyclopenta[c]pyridin-5-one (F-6)

nBuLi (16.9 g, 263 mmol) was added dropwise to a solution of F-5 (54.0 g, 176 mmol) in THF (800.0 mL) at −70° C. under N₂ over a period of 1 h. After addition, the resulting mixture was stirred at −70° C. for 1 h then carefully quenched with saturated aq. NH₄Cl (100 mL). The crude mixture was diluted with EtOAc (500 mL) and water (500 mL). The organic phase was separated and the aqueous was extracted with EtOAc (100 mL×3). The combined EtOAc layers were washed with brine (300 mL), then dried over Na₂SO₄, filtered and concentrated. The crude product was purified by column chromatography (220 g silica gel, 0-20% EtOAc/PE) to give F-6 (14.3 g, 49%) as gray solid. LCMS 168.3 [M+1]; ¹H NMR (400 MHz, CHLOROFORM-d) δ=8.49 (d, J=5.0 Hz, 1H), 7.54 (d, J=4.9 Hz, 1H), 3.31-3.14 (m, 2H), 2.85-2.71 (m, 2H).

### Step 6—Synthesis of 1-chloro-5-ethyl-6,7-dihydro-5H-cyclopenta[c]pyridin-5-ol (F-7)

CeCl₃ (3680 mg, 14.9 mmol) was heated under vacuum in a 250 mL flask at 130° C. for 20 min. This flask was cooled to 0-5° C., filled with argon then THF (40.0 mL) and EtMgBr (3980 mg, 29.8 mmol) were added dropwise to maintain the internal temperature at 0° C. The reaction was stirred for 1 hour at 0° C. then F-6 (2000 mg, 11.93 mmol) was added in a solution of THF (20.0 mL) maintaining the internal temperature at 0° C. The reaction was stirred for 1 h at 0° C. then quenched with saturated aq. NH₄Cl (40 mL) and diluted with EtOAc (40 mL). The resulting suspension was filtered and was washed with EtOAC (30 mL×3). The filtrate was separated, and the aqueous phase extracted with EtOAc (40 mL). The organic phases were washed with brine (30 mL), then dried over anhydrous Na₂SO₄, filtered and concentrated. The residue was purified by column chromatography (20 g silica gel, 0-27% EtOAc/PE) to give F-7 (1410 mg, 60%) as a yellow solid. ¹H NMR (400 MHz, CHLOROFORM-d) δ ppm 8.29 (d, J=5.02 Hz, 1H) 7.20 (d, J=5.02 Hz, 1H) 3.02-3.10 (m, 1H) 2.83-2.92 (m, 1H) 2.38 (ddd, J=13.49, 8.34, 4.02 Hz, 1H) 2.09-2.17 (m, 1H) 1.92 (s, 1H) 1.76-1.91 (m, 2H) 0.96 (t, J=7.53 Hz, 3H).

### Step 7—Synthesis of N-(1-chloro-5-ethyl-6,7-dihydro-5H-cyclopenta[c]pyridin-5-yl)acetamide (F-8)

To a solution of F-7 (1547 mg, 7.83 mmol) in CH₃CN (30.0 mL) was added H₂SO₄ (1540 mg, 15.7 mmol). The mixture was stirred at 60° C. for 16 h. The crude reaction was concentrated then diluted with ice-water (30 mL) and basified to pH=8-9 with sat. NaHCO₃ (15 mL). The mixture was extracted with EtOAc (30 mL×2). The combined extracts were washed with brine (30 mL) then dried (Na₂SO₄), filtered and concentrated. The crude product was purified by column chromatography (20 g Silica gel, 20-90% EtOAc/PE then 3-5% MeOH/EtOAc) to give F-8 (320 mg, 17%) as a red solid. LCMS 238.9 [M+1]; ¹H NMR (400 MHz, CHLOROFORM-d) δ ppm 8.24 (d, J=5.01 Hz, 1H) 7.14 (d, J=4.89 Hz, 1H) 5.70 (br s, 1H) 3.07-3.16 (m, 1H) 2.85-2.95 (m, 1H) 2.53 (ddd, J=13.36, 9.45, 7.03 Hz, 1H) 2.24 (ddd, J=13.24, 8.83, 4.10 Hz, 1H) 1.99-2.08 (m, 1H) 1.96 (s, 3H) 1.80 (dd, J=13.94, 7.34 Hz, 1H) 0.86 (t, J=7.46 Hz, 3H).

### Step 8—Synthesis of N-(1-chloro-5-ethyl-6,7-dihydro-5H-cyclopenta[c]pyridin-5-yl)-N-methylacetamide (F-9)

To a solution of F-8 (270 mg, 1.13 mmol) in THF (10 mL) was added NaH (163 mg, 6.79 mmol) at 0° C. After stirring for 0.5 h at 0° C., CH₃I (963 mg, 6.79 mmol) was added, and the reaction was warmed to rt and stirred for 2 h. The reaction was cooled to 0° C. and additional NaH (81.4 mg, 3.39 mmol) was added. After stirring for 0.5 h at 0° C. additional CH₃a (482 mg, 3.39 mmol) was added. The reaction was warmed to rt and stirred for 2 h then quenched with ice-water (15 mL) and extracted with EtOAc (15 mL×2). The combined extracts were washed with brine (15 mL), dried (Na₂SO₄), filtered and concentrated. The crude residue was purified by column chromatography (12 g silica gel, 90-100% EtOAc/PE) to give F-9 (240 mg, 84%) as a yellow solid. LCMS 253.0 [M+1].

### Step 9—Synthesis of N-(1-((diphenylmethylene)amino)-5-ethyl-6,7-dihydro-5H-cyclopenta[c]pyridin-5-yl)-N-methylacetamide (F-10)

To a solution of F-9 (240.0 mg, 0.95 mmol) in 1,4-dioxane (6 mL) was added benzophenone imine (344 mg, 1.90 mmol), Cs₂CO₃ (773 mg, 2.37 mmol), BINAP (73.9 mg, 0.119 mmol) and Pd(OAc)₂ (21.3 mg, 0.0950 mmol). The vial was purged with Argon for 4 min, then stirred at 100° C. for 14 h. The reaction was diluted with EtOAc (30 mL), then filtered and washed with EtOAc (10 mL×2). The filtrate was concentrated and purified by column chromatography (12 g silica gel, 0-100% EtOAc/PE) to give F-10 (380 mg, 100%) as yellow oil. LCMS 398.2 [M+1].

### Step 10—Synthesis of N(1-amino-5-ethyl-6,7-dihydro-5H-cyclopenta[c]pyridin-5-yl)acetamide (F-11)

To a solution of F-10 (380 mg, 0.96 mmol) in MeOH (10 mL) and DMA (2 mL) was added NH₂OH (101 mg, 1.53 mmol) followed by NaOAc (125 mg, 1.53 mmol). The mixture was stirred at 20° C. for 12 h. The reaction was concentrated to dryness. The residue was purified by column chromatography (12 g silica gel, 0-10% MeOH/DCM) to give F-11 (182 mg, 82%) as yellow oil. LCMS 234.1 [M+1].

### Synthesis of N-(3-chloro-5-ethyl-6,7-dihydro-5H-cyclopenta[c]pyridin-5-yl)-N,2-dimethylpropane-2-sulfinamide (G-11)

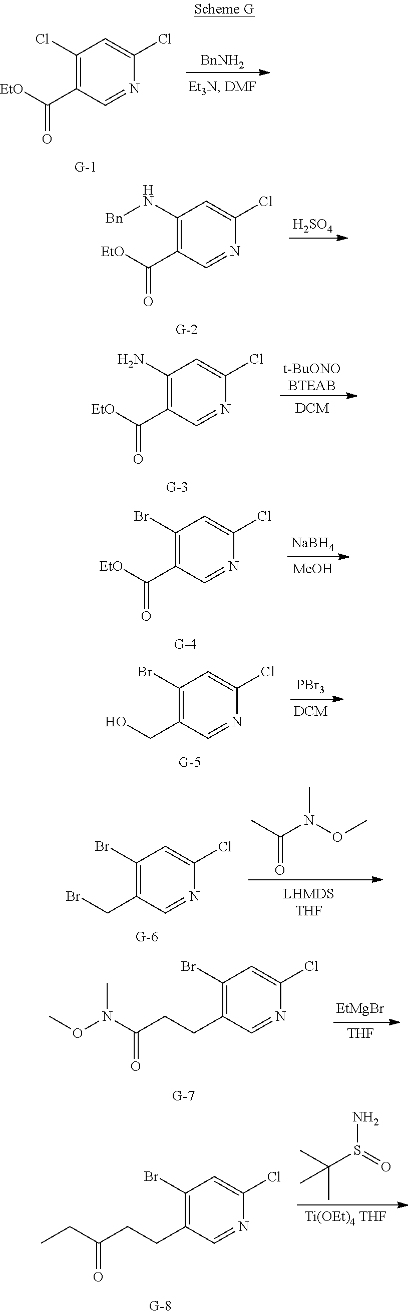

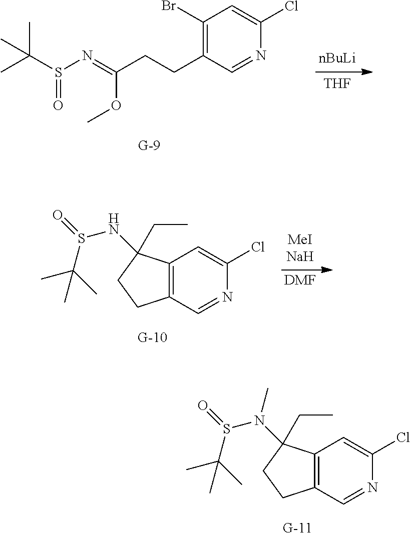

### Step 1—Synthesis of ethyl 4-(benzylamino)-6-chloronicotinate (G-2)

To a solution of G-1 (108 g, 492 mmol) in DMF (700 mL) was added Et₃N (149 g, 1480 mmol) and benzylamine (58.0 g, 541 mmol) at 25° C. The reaction was stirred at 25° C. for 3 h, then quenched with H₂O (1000 mL) and extracted with EtOAc (1000 mL×2). The extracts were washed with water (1000 mL) and brine (1000 mL), then dried (Na₂SO₄), filtered and concentrated to afford G-2 (145 g, 99%) as a yellow solid. LCMS 291.0 [M+1]; ¹H NMR (400 MHz, CHLOROFORM-d) δ ppm 8.71 (s, 1H) 8.57 (br s, 1H) 7.31-7.40 (m, 5H) 4.44 (d, 2H) 4.35 (q, 2H) 1.39 (t, 3H).

### Step 2—Synthesis of ethyl 4-amino-6-chloronicotinate (G-3)

To H₂SO₄ (450 g, 4590 mmol) was added G-2 (145 g, 499 mmol) in three portions at 5° C. The reaction was stirred at 5° C. for 1 hour then poured into cold water (1500 mL) and basified to pH=8-9 with solid Na₂CO₃ over 2 hours at 10° C. EtOAc (1500 mL) was added to the mixture and the resulting suspension was filtered. The filtrate was separated, and the aqueous layer was extracted with EtOAc (1000 mL). The combined organic layers were washed with brine (1500 mL), then dried (Na₂SO₄), filtered, concentrated. The crude product was re-crystalized from PE/EtOAc (600 mL/15 mL) to afford G-3 (87 g, 87%) as a yellow solid. LCMS 200.9 [M+1]; ¹H NMR (400 MHz, CHLOROFORM-d) δ ppm 8.70 (s, 1H) 6.57 (s, 1H) 4.34-4.40 (m, 2H) 1.38-1.42 (m, 3H).

### Step 3—Synthesis of ethyl 4-bromo-6-chloronicotinate (G-4)

To a solution of G-3 (40.0 g, 200 mmol) in DCM (1200 mL) was added BTEAB (163 g, 598 mmol) and t-BuONO (103 g, 997 mmol) at 0° C. The reaction was at 25° C. for 40 h. LCMS showed that SM remained, and product was detected. Additional BTEAB (130 g, 478 mmol) and t-BuONO (100 g, 970 mmol) were added at 0° C. The reaction was stirred at 25° C. for 18 h. The reaction was diluted with H₂O (2000 mL) and extracted with DCM (1000 mL). The organic phase was washed with brine (2000 mL), then dried (Na₂SO₄), filtered and concentrated. The crude product was purified by column chromatography (330 g silica gel, EtOAc/PE) to afford G-4 (26 g, 50%) as a yellow solid. LCMS 265.8 [M+1]; ¹H NMR (400 MHz, CHLOROFORM-d) δ ppm 8.81 (s, 1H) 7.69 (s, 1H) 4.44 (q, 2H) 1.43 (t, 3H).

### Step 4—Synthesis of (4-bromo-6-chloropyridin-3-yl)methanol (G-5)

To a solution of G-4 (44.75 g, 169.2 mmol) in DCM (500 mL) was added DIBAL-H (70 g, 500 mmol) at −70° C. After stirring for 30 minutes at −70° C., the reaction was warmed to 25° C. and stirred for 60 min. The reaction was poured into 3N HCl (500 mL). The suspension was extracted with DCM (500 mL×2). The combined organic layers were washed with sat. Na₂CO₃ (500 mL), then dried (Na₂SO₄), filtered and concentrated. The crude product was purified by re-crystalized from (EtOAc/PE=10 mL/200 mL) to afford G-5 (22 g, 59%) as a yellow solid. LCMS 223.7 [M+1]; ¹H NMR (400 MHz, CHLOROFORM-d) δ ppm 8.45 (s, 1H) 7.57 (s, 1H) 4.79 (s, 2H).

### Step 5—Synthesis of 4-bromo-5-(bromomethyl)-2-chloropyridine (G-6)

To a solution of G-5 (39.0 g, 175.3 mmol) in DCM (500.0 mL) was added PBr₃ (57.0 g, 211 mmol) at 0° C. The reaction was warmed to 25° C. and stirred for 18 h. The reaction mixture was diluted with ice-water (500 mL) and extracted with DCM (400 mL×2). The combined organic layers were washed with sat. Na₂CO₃ (400 mL), then dried (Na₂SO₄), filtered, concentrated. The crude residue was purified by column chromatography (330 g silica gel, 8-20% EtOAc/PE) to afford G-6 (40.3 g, 81%) as a white solid. LCMS 285.9 [M+1]; ¹H NMR (400 MHz, CHLOROFORM-d) δ ppm 8.42 (s, 1H) 7.61 (s, 1H) 4.55 (s, 2H).

### Step 6—Synthesis of 3-(4-bromo-6-chloropyridin-3-yl)-N-methoxy-N-methylpropanamide (G-7)

A solution of N-methoxy-N-methylacetamide (32.9 g, 319 mmol) in THF (500 mL) was cooled to −78° C. then LHMDS (53.4 g, 319 mmol) was added dropwise while the temperature was maintained between −70° C. and −78° C. After addition, the reaction mixture was stirred at −78° C. for 60 min. A solution of G-6 (45.50 g, 159.4 mmol) in THF (200 mL) was added dropwise while maintaining the temperature between at −70° C. and −78° C. The reaction was stirred at −78° C. for 1 h. Saturated aqueous NH₄Cl (500 mL) was added dropwise to the reaction mixture, then diluted with H₂O (500 mL) and extracted with EtOAc (1000 mL×2). The combined organic layers were washed with brine (1000 mL), then dried (Na₂SO₄), filtered and concentrated. The crude product was purified by column chromatography (330 g silica gel, 10-30% EtOAc/PE) to afford G-7 (21.5 g, 44%) as a yellow solid. LCMS 307.3, 309.0 [M+1]; 1H NMR (400 MHz, CHLOROFORM-d) δ ppm 8.28 (s, 1H) 7.55 (s, 1H) 3.66 (s, 3H) 3.18 (s, 3H) 3.04-3.10 (m, 2H) 2.77 (br t, 2H).

### Step 7—Synthesis of 1-(4-bromo-6-chloropyridin-3-yl)pentan-3-one (G-8)

A mixture of G-7 (3130 mg, 10.18 mmol) in THF (50.0 mL) was cooled to −20° C. then EtMgBr (2980 mg, 22.4 mmol) was added. After the addition, the mixture was stirred at −20° C. for 1 h. TLC indicated starting material remained and additional EtMgBr (800 mg, 6 mmol) was added at −20° C. and the reaction was stirred for 1 h. Saturated aqueous NH₄Cl (60 mL) was added dropwise to the reaction mixture at −20° C., then diluted with H₂O (40 mL) and extracted with EtOAc (60 mL×2). The combined organic layers were washed with brine (60 mL), then dried (Na₂SO₄), filtered and concentrated. The crude residue was purified by column chromatography (20 g silica gel, 20-45% EtOAc/PE) to afford G-8 (1900 mg, 68%) as a yellow solid. LCMS 275.9 [M+1].

### Step 8—Synthesis of (E)-N-(1-(4-bromo-6-chloropyridin-3-yl)pentan-3-ylidene)-2-methylpropane-2-sulfinamide (G-9)

To a solution of G-8 (2010 mg, 7.268 mmol) in THF (50 mL) was added 2-methylpropane-2-sulfinamide (1320 mg, 10.9 mmol) and Ti(OEt)₄ (4970 mg, 21.8 mmol) at 25° C. The reaction was stirred at 80° C. for 15 h. The reaction was diluted with EtOAc (100 mL) and sat. NaHCO₃ (50 ml), then filtered through Celite. The filtrate was extracted with EtOAc (50 mL×2) and the combined organic layers were washed with brine (50 ml), then dried over Na₂SO₄, filtered and concentrated. The crude residue was purified by column chromatography (20 g silica gel, 15-35% EtOAc/PE) to give G-9 (2400 mg, 87%) as yellow oil. LCMS 380.8 [M+1]; ¹H NMR (400 MHz, CHLOROFORM-d) δ ppm 8.18-8.30 (m, 1H) 7.56 (s, 1H) 2.93-3.08 (m, 3H) 2.66-2.82 (m, 3H) 1.26 (s, 9H) 1.20-1.25 (m, 3H).

### Step 9—Synthesis of N-(3-chloro-5-ethyl-6,7-dihydro-5H-cyclopenta[c]pyridin-5-yl)-2-methylpropane-2-sulfinamide (G-10)

A mixture of G-9 (2760 mg, 7.268 mmol) in THF (40 mL) was cooled to −78° C., then nBuLi (698 mg, 10.9 mmol) was added dropwise under N₂. The reaction was stirred at −78° C. for 30 min then quenched with saturated aqueous NH₄Cl (40 mL). The mixture was diluted with H₂O (40 mL) and extracted with EtOAc (60 mL×2). The combined organic layers were washed with brine (60 mL), then dried (Na₂SO₄), filtered and concentrated. The crude residue was purified by column chromatography (20 g silica gel, 35-80% EtOAc/PE) to afford G-10 (800 mg, 37%) as yellow oil. LCMS 301.0 [M+1]; ¹H NMR (400 MHz, CHLOROFORM-d) δ ppm 8.25 (d, 1H) 7.45-7.67 (m, 1H) 7.25 (s, 1H) 2.90-3.04 (m, 3H) 2.70-2.84 (m, 2H) 2.44 (br dd, 1H) 1.25 (br d, 9H) 1.17-1.22 (m, 3H).

### Step 10—Synthesis of N-(3-chloro-5-ethyl-6,7-dihydro-5H-cyclopenta[c]pyridin-5-yl)-N,2-dimethylpropane-2-sulfinamide (G-11)

To a solution of G-10 (600.0 mg, 1.99 mmol) in DMF (15 mL) was slowly added NaH (128 mg, 3.19 mmol) at 0° C. After stirring for 0.5 h, MeI (368 mg, 2.59 mmol) was added at 0° C., and the reaction was warmed to 25° C. and stirred for 3 h. The reaction was quenched with ice-water (20 mL) and extracted with EtOAc (40 mL×2). The combined organic layers were washed with brine (30 mL×2), then dried over Na₂SO₄, filtered and concentrated. The crude residue was purified by column chromatography (24 g silica gel, 10-40% EtOAc/PE) to give G-11 (130 mg, 16%) as yellow oil. LCMS 315.0 [M+1].

### Synthesis of (R)—N—((R)-2-(2-chloropyridin-4-yl)butan-2-yl)-2-methylpropane-2-sulfinamide (H-5)

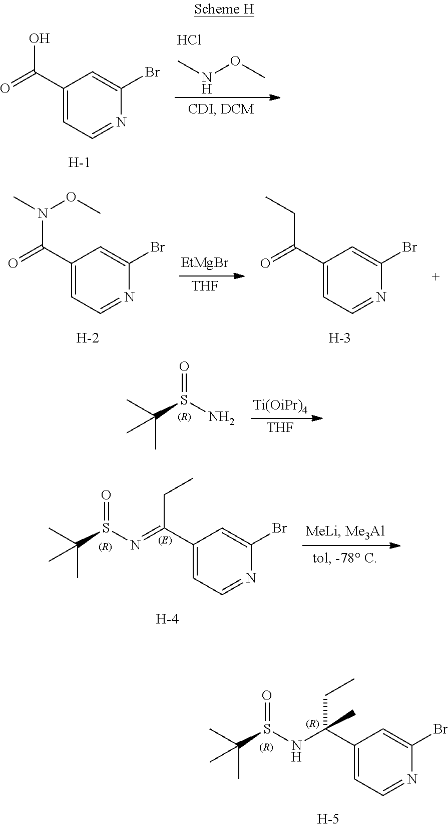

### Step 1—Synthesis of 2-bromo-N-methoxy-N-methylisonicotinamide (H-2)

To a solution of H-1 (250 g, 1.24 mmol) in DCM (2500 mL) was added 1,1′-carbonyldiimidazole (220 g, 1.36 mol, 1.1 eq) in multiple portions. The mixture was stirred at 25° C. for 2 hours, then N-methoxymethanamine-hydrochloride (181 g, 1.86 mol) was added in multiple portions. The reaction was stirred at 25° C. for 14 h. The reaction was quenched with 0.1N NaOH (1000 mL) then water (200 mL) and DCM (1000 mL) were added. The layers were separated and the aqueous phase was back-extracted with DCM (1000 mL×2). The combined organic layers were washed with brine (500 mL), dried over Na₂SO₄, then filtered and concentrated to give crude H-2 (885 g, crude) as a yellow oil. The crude product was used into the next step without further purification. LCMS 247.0, 245.0 [M+1]; ¹H NMR (400 MHz, CHLOROFORM-d) δ=8.50-8.42 (m, 1H), 7.71 (s, 1H), 7.48 (dd, J=1.3, 5.0 Hz, 1H), 3.55 (s, 3H), 3.36 (s, 3H).

### Step 2—Synthesis of 1-(2-bromopyridin-4-yl)propan-1-one (H-3)

A solution of H-2 (240 g, 980.5 mmol) in THF (2.0 L) was purged with N₂ (3 times). The solution was cooled to −10° C. and was slowly added EtMgBr (3 M, 780 mL) keeping temperature at −10° C. to −5° C. The mixture was stirred at −10° C. to −5° C. for 1.5 h. The reaction mixture was slowly poured into sat NH₄Cl (1.2 L) then extracted with EtOAc (1000 mL×2). The combined organic layers were washed with brine (800 mL), dried over Na₂SO₄ then filtered and concentrated. The crude residue was purified by column chromatography (SiO₂, Petroleum ether/Ethyl acetate=10/1 to 5/1) to give H-3 (420 g, 51%) as yellow oil. LCMS 216.0, 214.0 [M+1]; ¹H NMR (400 MHz, CHLOROFORM-d) δ=8.54 (d, J=5.0 Hz, 1H), 7.93-7.89 (m, 1H), 7.68 (dd, J=1.4, 5.1 Hz, 1H), 2.98 (q, J=7.2 Hz, 2H), 1.24 (t, J=7.2 Hz, 3H)

### Step 3—Synthesis of (R,E)-N-(1-(2-chloropyridin-4-yl)propylidene)-2-methylpropane-2-sulfinamide (H4)

A solution of H-3 (130 g, 607 mmol), (R)-2-methylpropane-2-sulfinamide (147 g, 1.21 mol) and titanium isopropoxide (345 g, 1.21 mol) in THF (1000 mL) was degassed and purged with N₂ 3 times, and the mixture was stirred at 75° C. for 8 hours under a N₂ atmosphere. After cooling, the reaction was quenched with aq. NaHCO₃ (1500 mL) with stirring until a white precipitate formed. The solid was filtered through a Celite pad. The filtrate was washed with brine (500 mL), dried over anhydrous Na₂SO₄ then concentrated to give crude product. The crude residue was purified by column chromatography (SiO₂, Petroleum ether/Ethyl acetate=100/1 to 50/1) to give H-4 (90 g, 47%) as a yellow oil. LCMS 319.0, 317.0 [M+1]; ¹H NMR (400 MHz, CHLOROFORM-d) δ=8.47 (d, J=5.1 Hz, 1H), 7.80 (br s, 1H), 7.58 (br s, 1H), 3.21 (br s, 2H), 1.34 (s, 9H), 1.29-1.23 (m, 3H).

### Step 4—Synthesis of (R)—N—((R)-2-(2-chloropyridin-4-yl)butan-2-yl)-2-methylpropane-2-sulfinamide (H-5)

To a solution of H4 (500 mg, 1.58 mmol) in toluene (5.0 mL) was added C₃H₉Al (125 mg, 1.73 mmol, 2 M in toluene) at −78° C. The resulting solution was stirred for 5 min before it was added over the course of 20 min to −78° C. solution of MeLi (76.2 mg, 3.47 mmol) in toluene (5 mL). The reaction was stirred at −78° C. for 3 h, then warmed to 0° C. The reaction was treated with aq. Na₂SO₄, then stirred at rt for 5 min. Then solid Na₂SO₄ was added and the mixture was filter and rinsed with toluene. The filtrate was concentrated and purified by column chromatography (20 g silica gel, 0-100% EtOAc/PE) to give H-5 (361.1 mg, 69%) as a yellow oil. LCMS 335.1 [M+1]; ¹H NMR (400 MHz, CHLOROFORM-d) δ=8.35 (d, J=5.1 Hz, 1H), 7.55 (d, J=1.2 Hz, 1H), 7.35 (dd, J=1.6, 5.3 Hz, 1H), 3.48 (s, 1H), 2.01-1.94 (m, 2H), 1.67 (s, 3H), 1.27 (s, 9H), 0.79 (t, J=7.3 Hz, 3H).

### Synthesis of (S)—N—((S)-2-(2-bromopyridin-4-yl)butan-2-yl)-2-methylpropane-2-sulfinamide (I-1)

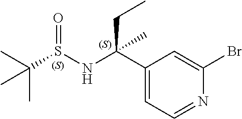

Intermediate I-1 was made in a similar fashion to H-5 substituting (S)-2-methylpropane-2-sulfinamide for (R)-2-methylpropane-2-sulfinamide in Scheme H, Step 3. LCMS 335.1 [M+1]; ¹H NMR (400 MHz, DMSO-d6) δ=8.32 (d, J=5.3 Hz, 1H), 7.75 (d, J=1.0 Hz, 1H), 7.53 (dd, J=1.8, 5.3 Hz, 1H), 5.64 (s, 1H), 1.84 (q, J=7.4 Hz, 2H), 1.53 (s, 3H), 1.17 (s, 9H), 0.73 (t, J=7.3 Hz, 3H).

### Synthesis of (R)—N—((S)-1-(2-bromopyridin-4-yl)propyl)-N,2-dimethylpropane-2-sulfinamide (J-2)

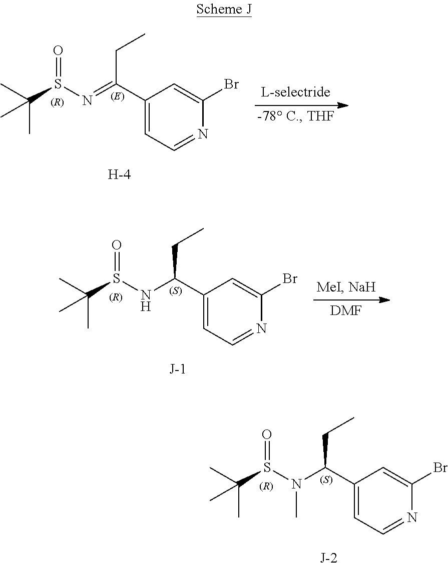

### Step 1—Synthesis of (R)—N—((S)-1-(2-bromopyridin-4-yl)propyl)-2-methylpropane-2-sulfinamide (J-1)

To a solution of H-4 (4060 mg, 12.80 mmol) in THF (64 mL) was added L-selectride (14600 mg, 76.8 mmol) dropwise at −10° C. The mixture was stirred at 10° C. for 16 h then quenched with NH₄Cl (40 mL) and stirred for 3 h. The reaction mixture was extracted with EtOAc (50 mL×3). The combined organic layers were dried over Na₂SO₄, filtered and concentrated. The crude residue was purified by column chromatography (20 g silica gel, 30-50% EtOAc/PE) to give J-1 (1700 mg, 42%) as a yellow gum. ¹H NMR (400 MHz, METHANOL-d4) δ=8.30 (d, J=5.3 Hz, 1H), 7.61 (s, 1H), 7.38 (dd, J=1.3, 5.1 Hz, 1H), 4.26 (t, J=7.1 Hz, 1H), 1.99-1.88 (m, 1H), 1.88-1.74 (m, 1H), 1.20 (s, 9H), 0.93 (t, J=7.4 Hz, 3H).

### Step 2—Synthesis of (R)—N—((S)-1-(2-bromopyridin-4-yl)propyl)-N,2-dimethylpropane-2-sulfinamide (J-2)

To a solution of J-1 (1700 mg, 5.325 mmol) in anhydrous DMF (30.0 mL) was added NaH (319 mg, 7.99 mmol) at 0° C. The mixture was stirred at 0° C. for 30 min, then MeI (983 mg, 6.92 mmol) was added. The cold bath was removed, and the reaction was warmed to rt for 16 h. The reaction mixture was quenched with NH₄Cl aq. (10 mL) and extracted with EtOAc (20 mL×3). The combined with organic layers were washed with water (15 mL×2) and brine (15 mL×4), then dried over Na₂SO₄, filtered and concentrated. The crude residue was purified by column chromatography (20 g silica gel, 0-70% EtOAc/PE) to give J-2 (310 mg, 18%, purity) as a yellow oil. LCMS 335.1, 333.1 [M+1]; 1H NMR (400 MHz, CHLOROFORM-d) δ 8.27-8.46 (m, 1H), 7.47 (s, 1H), 7.27-7.28 (m, 1H), 4.09-4.26 (m, 1H), 2.51 (s, 3H), 1.92-2.06 (m, 2H), 1.15-1.27 (m, 9H), 0.95-1.01 (m, 3H).

### Synthesis of (S)—N—((R)-1-(2-bromopyridin-4-yl)propyl)-N,2-dimethylpropane-2-sulfinamide (K-1)

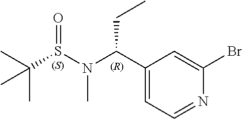

K-1 was made in a similar fashion to J-2 substituting (S,E)-N-(1-(2-bromopyridin-4-yl)propylidene)-2-methylpropane-2-sulfinamide for H-4 as the starting material in Scheme J, Step 1. LCMS 335.1, 333.1 [M+1]; ¹H NMR (400 MHz, CHLOROFORM-d) δ 8.38 (d, J=5.13 Hz, 1H), 7.47 (s, 1H), 7.28-7.29 (m, 1H), 4.18 (t, J=7.57 Hz, 1H), 2.51 (s, 3H), 1.93-2.08 (m, 2H), 1.19-1.28 (m, 9H), 0.98 (t, J=7.38 Hz, 3H).

### Synthesis of (R)—N—((R)-2-(2-chloro-5-methylpyridin-4-yl)butan-2-yl)-2-methylpropane-2-sulfinamide (L-4)

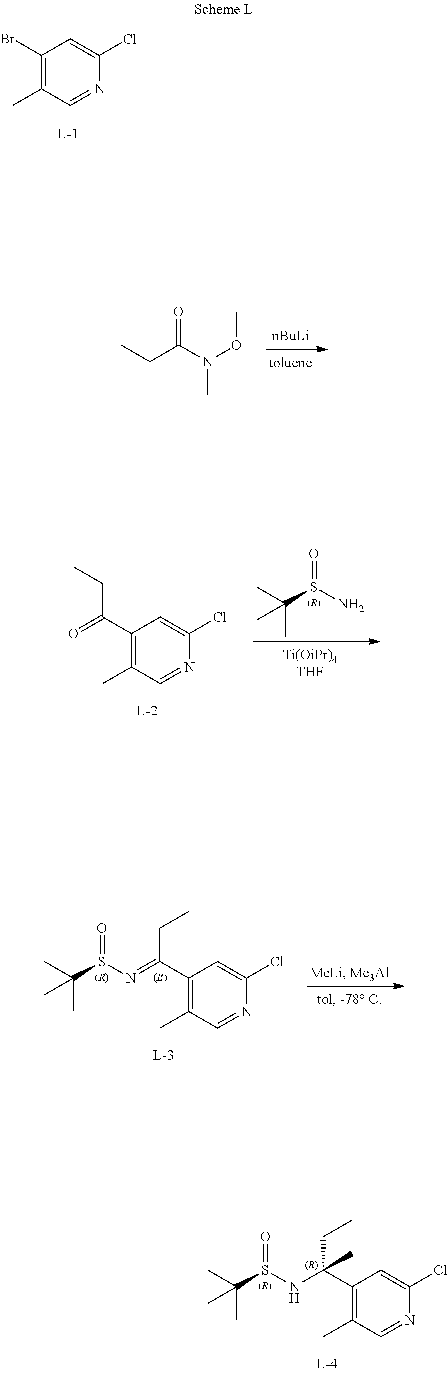

Step 1—Synthesis of 1-(2-chloro-5-methylpyridin-4-yl)propan-1-one (L-2) To a solution of L-1 (3500.0 mg, 16.95 mmol) in anhydrous toluene (150 mL) was added nBuLi (1740 mg, 27.1 mmol, 2.5 M) dropwise at −65° C. The mixture was stirred for 30 min at −65° C. then N-Methoxy-N-methyl propionamide (2380 mg, 20.3 mmol) in anhydrous toluene (10 mL) was added dropwise at −65° C., over 30 min. After addition, the mixture was stirred at −65° C. for 3 h then warmed to rt and stirred for an additional 12 h. The crude reaction was quenched with sat. NH₄Cl and extracted with EtOAc. The organic layer was washed with water and brine, then dried over Na₂SO₄ and concentrated. The crude residue was purified by column chromatography (20 g silica gel, 0-10% EtOAc/PE) to give L-2 (1700 mg, 55%) as a thick yellow thick oil which solidified on standing. LCMS 184.0 [M+1]; ¹H NMR (400 MHz, CHLOROFORM-d) δ=8.31 (s, 1H), 7.41 (s, 1H), 2.88 (q, J=7.2 Hz, 2H), 2.39 (s, 3H), 1.21 (t, J=7.2 Hz, 3H).

### Step 2—Synthesis of (R,E)-N-(1-(2-chloro-5-methylpyridin-4-yl)propylidene)-2-methylpropane-2-sulfinamide (L-3)

L-3 was made in a similar fashion to H-4 (Scheme H) using 1-(2-chloro-5-methylpyridin-4-yl)propan-1-one (L-2) in place of 2-bromo-N-methoxy-N-methylisonicotinamide (H-2). LCMS 287.0 [M+1]; ¹H NMR (400 MHz, METHANOL-d4) δ=8.35-8.19 (m, 1H), 7.36-7.24 (m, 1H), 2.89-2.58 (m, 2H), 2.39-2.19 (m, 3H), 1.32-1.21 (m, 12H).

### Step 3—Synthesis of (R)—N—((R)-2-(2-chloro-5-methylpyridin-4-yl)butan-2-yl)-2-methylpropane-2-sulfinamide (L-4)

L-4 was made in a similar fashion to H-5 (Scheme H) using (R,E)-N-(1-(2-chloro-5-methylpyridin-4-yl)propylidene)-2-methylpropane-2-sulfinamide (L-3) in place of (R,E)-N-(1-(2-chloropyridin-4-yl)propylidene)-2-methylpropane-2-sulfinamide (H-4). LCMS 303.0 [M+1].

### Synthesis of (S)—N—((S)-2-(2-chloro-5-methylpyridin-4-yl)butan-2-yl)-2-methylpropane-2-sulfinamide (M-1)

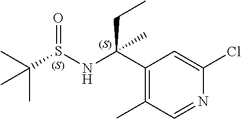

Intermediate M-1 was made in a similar fashion to L-4 substituting (S)-2-methylpropane-2-sulfinamide for (R)-2-methylpropane-2-sulfinamide in Scheme L, Step 2. LCMS 303.0 [M+1].

### Synthesis of N-(2-(2-chloropyridin-4-yl)butan-2-yl)-N,2-dimethylpropane-2-sulfinamide (N-4)

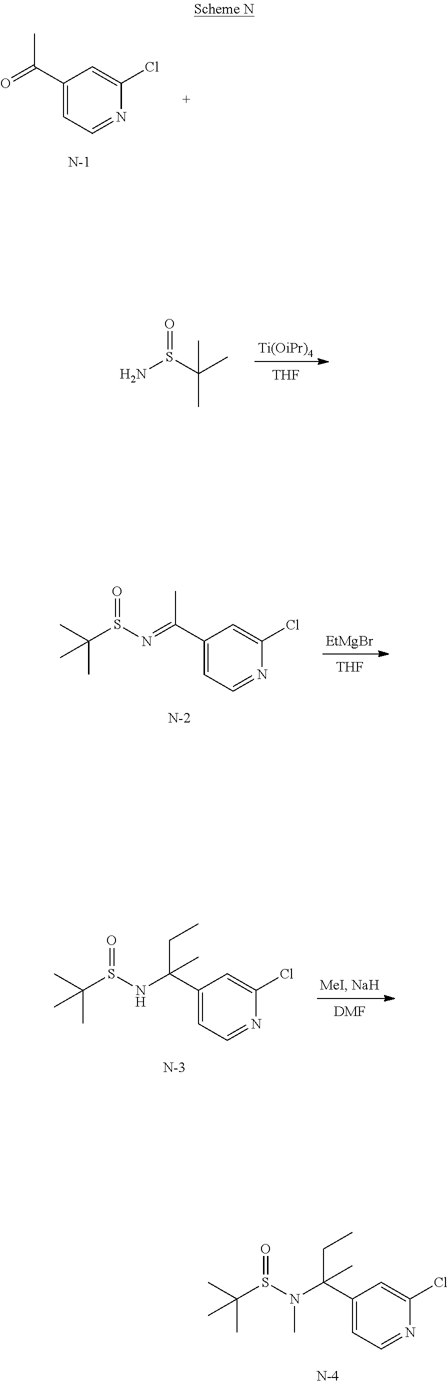

### Step 1—Synthesis of (E)-N-(1-(2-chloropyridin-4-yl)ethylidene)-2-methylpropane-2-sulfinamide (N-2)

N-2 was made in a similar fashion to H-4 substituting 1-(2-chloropyridin-4-yl)ethan-1-one (N-1) and racemic 2-methylpropane-2-sulfinamide for 1-(2-bromopyridin-4-yl)propan-1-one (H-3) and (R)-2-methylpropane-2-sulfinamide. LCMS 258.9 [M+1]; ¹H NMR (400 MHz, DMSO-d6) δ=8.56 (d, J=5.1 Hz, 1H), 7.87-7.73 (m, 2H), 5.15-5.04 (m, 1H), 2.73 (s, 3H), 1.23 (s, 9H).

### Step 2—Synthesis of N-(2-(2-chloropyridin-4-yl)butan-2-yl)-2-methylpropane-2-sulfinamide (N-3)

To a solution of EtMgBr (2470 mg, 18.5 mmol, 3 M in Et₂O) in THF (20 mL) was added N-2 (2400 mg, 9.275 mmol) at −20° C. The mixture was stirred at 15° C. for 2 h. The reaction was quenched with NH₄Cl (20 mL) and extracted with EtOAc (50 mL×3). The combined organic layers were washed with water (20 mL×2) and brine (20 mL), then dried over Na₂SO₄, filtered and concentrated. The residue was purified by column chromatography (40 g silica gel, 0-30% EtOAc/PE) to give N-3 (570 mg, 21%) as a yellow oil. LCMS 289.0 [M+1]; ¹H NMR (400 MHz, DMSO-d6) δ=8.56 (d, J=5.1 Hz, 1H), 8.39-8.31 (m, 1H), 7.87-7.74 (m, 2H), 1.96-1.75 (m, 2H), 1.24 (s, 9H), 1.18-1.16 (m, 3H), 0.77-0.61 (m, 3H).

### Step 3—Synthesis of N-(2-(2-chloropyridin-4-yl)butan-2-yl)-N,2-dimethylpropane-2-sulfinamide (N-4)

To a solution of N-3 (420.0 mg, 1.45 mmol) in DMF (5.0 mL) was added NaH (87.2 mg, 2.18 mmol) slowly at 0° C. After 0.5 h, MeI (310 mg, 2.18 mmol) was added, and the reaction was stirred at 25° C. for 3 hours. The reaction was quenched with sat. NH₄Cl (5 mL) and extracted with EtOAc (5 mL×2). The combined the organic layers were washed with brine (5 mL×2), dried over Na₂SO₄, then filtered and concentrated. The crude residue was purified by column chromatography (12 g silica gel, EtOAc/PE=0/1 to 1/1) to give N-4 (220 mg, 45%) as a colorless oil. LCMS 302.9 [M+1].

### Synthesis of 2-(2-chloropyridin-4-yl)-N,N-dimethylpropan-2-amine (0-4)

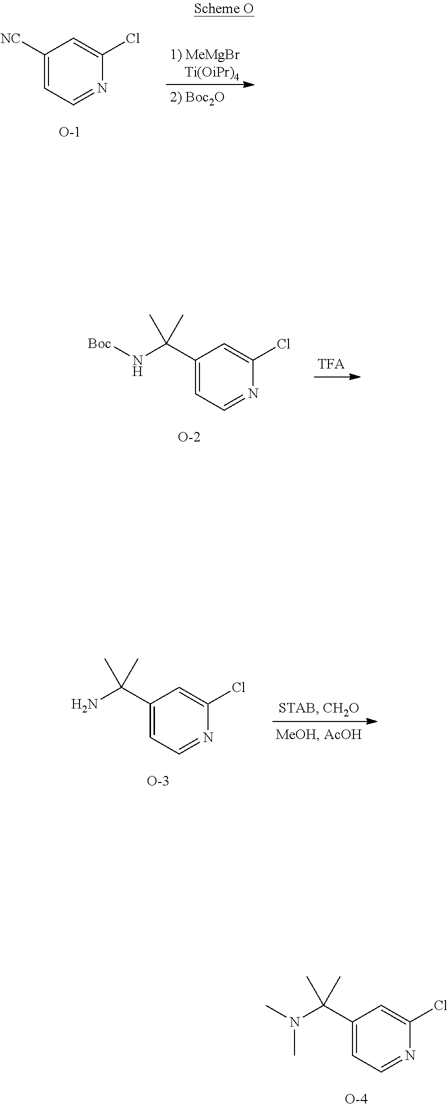

Step 1—Synthesis of tert-butyl (2-(2-chloropyridin-4-yl)propan-2-yl)carbamate (0-2) To a mixture of 0-1 (20000.0 mg, 144.35 mmol) in THF (560 mL) was slowly added EtMgBr (51600 mg, 433 mmol) at 0° C. The reaction was warmed to 20° C. for 40 mins then Ti(OiPr)₄ (61500 mg, 173 mmol) was added and stirring was continued at 55° C. for 18 h. The crude reaction was diluted with sat. NaHCO₃ (300 mL) then Boc₂O (41000 mg, 188 mmol) was added and the reaction was stirred for 36 h. The suspension was filtered washed with EtOAc (800 mL). The filtrate was separated, and the organic phase was washed with brine (200 mL×2) then dried over Na₂SO₄, filtered and concentrated. The crude product was purified by column chromatography (330 g silica gel, 0-20% EtOAc/PE) to give 0-2 (8.8 g, 23%) as a light brown solid. LCMS 271.2 [M+1]; ¹H NMR (400 MHz, CHLOROFORM-d) δ=8.31 (d, J=5.1 Hz, 1H), 7.31 (s, 1H), 7.21 (dd, J=1.3, 5.2 Hz, 1H), 4.98 (br s, 1H), 1.58 (s, 6H), 1.44-1.15 (m, 9H).

### Step 2—Synthesis of 2-(2-chloropyridin-4-yl)propan-2-amine (0-3)

To a solution of 0-2 (500 mg, 1.85 mmol) in MeOH (1 mL) was added 4 M HCl in 1,4-dioxane (5 mL, 20 mmol). The reaction was stirred at 28° C. for 18 h. To the reaction mixture was added Et₃N until the pH was adjusted to 8. The reaction was concentrated then diluted EtOAc and a small amount of water. The mixture was with extracted with EtOAc (10 mL×3) then the combined organic layers were dried over Na₂SO₄, filtered and concentrated to give 0-3 (290 mg, 92%) as a yellow oil. ¹H NMR (400 MHz, DMSO-d6) δ ppm 1.35 (s, 6H) 7.53 (dd, J=5.32, 1.65 Hz, 1H) 7.63 (d, J=1.10 Hz, 1H) 8.31 (d, J=4.89 Hz, 1H).

### Step 3—Synthesis of 2-(2-chloropyridin-4-yl)-N,N-dimethylpropan-2-amine (0-4)

To a solution of 0-3 (150 mg, 0.879 mmol) and HCHO (713 mg, 8.79 mmol) in MeOH (3.52 mL) was added HCOOH (40.5 mg, 0.879 mmol) at 20° C. After stirring for 3 h, NaBH(AcO)₃ (559 mg, 2.64 mmol) was added at 0° C. and the resulting mixture was stirred at 20° C. for 15 h. The crude reaction was adjusted to pH 8 with saturated Na₂CO₃ (2 mL) then concentrated. The crude compound was purified by column chromatography (20 g silica gel, 0-5% MeOH/DCM) to give 0-4 (150 mg, 86%) as a yellow oil. LCMS 199.3 [M+1]; ¹H NMR (400 MHz, CHLOROFORM-d) δ ppm 1.22-1.42 (m, 6H) 1.51-1.70 (m, 6H) 7.35-7.44 (m, 1H) 7.45-7.60 (m, 1H) 8.14-8.51 (m, 1H).

### Synthesis of tert-butyl (2-(2-chloropyridin-4-yl)propan-2-yl)(methyl)carbamate (P-1)

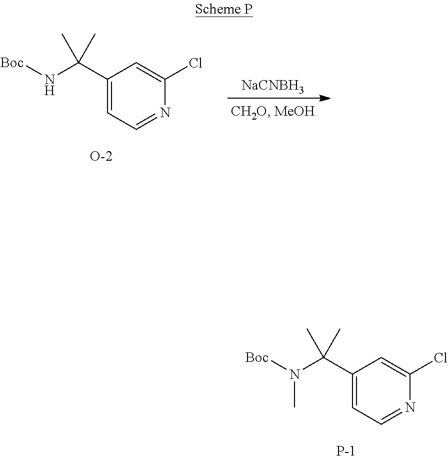

To a solution of 0-2 (300 mg, 1.11 mmol) in THF (12 mL) was added NaH (84.1 mg, 2.11 mmol) at 0° C. The mixture was stirred for 0.5 h then CH₃I (312 mg, 2.28 mmol) was added at 0° C. and stirring was continued for 16 h at 25° C. The reaction mixture was poured into water and extracted with EtOAc (2×20 mL). The organic phases were combined, washed with water, then dried over sodium sulfate, filtered and concentrated. The crude residue was purified by column chromatography (0-5% MeOH in DCM) to give P-1 (230 mg, 73.0%) as a yellow oil. LCMS 285.2 [M+1]; ¹H NMR (400 MHz, CHLOROFORM-d) δ=8.28 (d, J=5.3 Hz, 1H), 7.23 (d, J=1.3 Hz, 1H), 7.13 (dd, J=1.6, 5.4 Hz, 1H), 3.08 (s, 3H), 1.56 (s, 6H), 1.16 (s, 9H).

### Synthesis of N-(1-(2-chloro-5-methylpyridin-4-yl)propyl)-2-methylpropane-2-sulfinamide (Q-2)

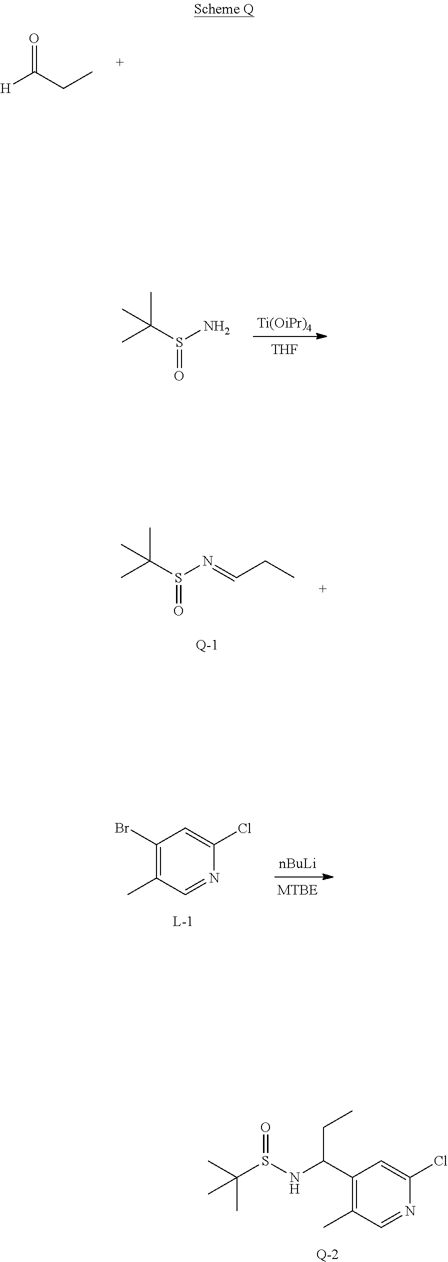

### Step 1—Synthesis of (E)-2-methyl-N-propylidenepropane-2-sulfinamide (Q-1)

To a solution of propionaldehyde (8630 mg, 149 mmol) and titanium isopropoxide (70300 mg, 248 mmol) in THF (309 mL) was added 2-methylpropane-2-sulfinamide (15000 mg, 123.76 mmol). The mixture was stirred at 50° C. for 16 h, then cooled to rt and poured into brine (250 mL). The mixture was filtered through Celite and rinsed with EtOAc. The organic layer was separated and washed with brine, then dried over Na₂SO₄, filtrated, and concentrated. The crude residue was purified by column chromatography (120 g silica gel, 0-15% EtOAc/PE) to afford Q-1 (12.5 g, 63%) as yellow oil. LCMS 161.9 [M+1]; ¹H NMR (400 MHz, CHLOROFORM-d) δ=8.03 (t, J=4.3 Hz, 1H), 2.47 (dq, J=4.3, 7.4 Hz, 2H), 1.12 (s, 9H), 1.12-1.07 (m, 3H).

### Step 2—Synthesis of N-(1-(2-chloro-5-methylpyridin-4-yl)propyl)-2-methylpropane-2-sulfinamide (Q-2)

All flasks used in the reaction were heated under vacuum for 10 minutes and purged with Argon for 4 times. To a solution of L-1 (768 mg, 3.72 mmol) in anhydrous MTBE (8.3 mL) was added nBuLi (238 mg, 3.72 mmol) dropwise at −67° C. After addition the reaction was stirred for 10 min, then a solution of Q-1 (300 mg, 1.86 mmol) in MTBE (1 mL) was added dropwise. The cold bath was removed, and the reaction was warmed to rt over 1.5 h. The reaction was quenched with sat. NH₄4 (10 mL) and extracted with EtOAc (25 mL). The organic phase was dried over Na₂SO₄, filtrated, and concentrated. The cure residue was purified by column chromatography (12 g silica gel, 0-10% MeOH/DCM) to afford Q-2 (537 mg, 99%) as yellow gum. LCMS 289.1 [M+1]; ¹H NMR (400 MHz, CHLOROFORM-d) δ=8.17 (s, 1H), 7.38-7.24 (m, 1H), 4.52-4.46 (m, 1H), 4.93-4.29 (m, 1H), 2.52-2.19 (m, 3H), 1.90-1.67 (m, 2H), 1.35-1.08 (m, 9H), 0.98-0.82 (m, 3H).

### Synthesis of N-((2-chloropyridin-4-yl)cyclopropyl)methyl)-N,2-dimethylpropane-2-sulfinamide (R-3)

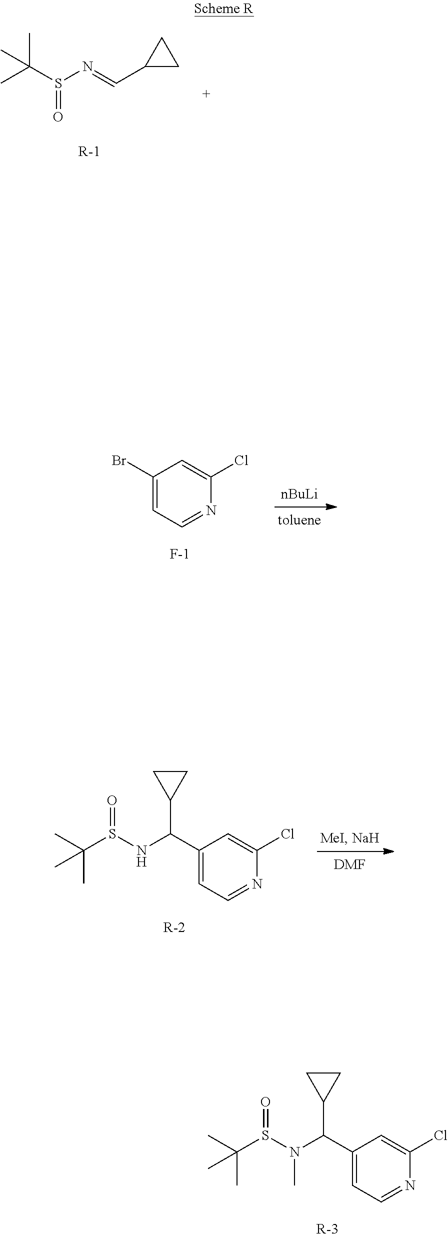

### Synthesis of (E)-N-(cyclopropylmethylene)-2-methylpropane-2-sulfinamide (R-1)

R-1 was made in a similar fashion to Q-1 using cyclopropanecarbaldehyde in place of propionaldehyde. (Scheme Q, Step 1)

### Step 1—Synthesis of N-((2-chloropyridin-4-yl)cyclopropyl)methyl)-2-methylpropane-2-sulfinamide (R-2)

To a solution of 4-bromo-2-chloropyridine (F-1) (1440 mg, 7.50 mmol) in anhydrous toluene (18.0 mL) was added nBuLi (555 mg, 8.66 mmol, 2.5 M in hexane) dropwise at −65° C. After 30 min, R-1 (1000 mg, 5.77 mmol) in anhydrous toluene (11.0 mL) was added dropwise and stirred at −65° C. for 10 min. The cold bath was removed, and the reaction was warmed to rt over 1.5 h. The mixture was quenched with sat. NH₄Cl and extracted with EtOAc. The organic layer was washed with water and brine, then dried over Na₂SO₄ and concentrated. The crude residue was purified by column chromatography (40 g silica gel, 0-100% EtOAc/PE) to afford R-2 (1.25 g, 76%) as a brown oil. LCMS 287.3 [M+1]; ¹H NMR (400 MHz, DMSO-d6) δ ppm 0.38-0.54 (m, 3H) 0.57-0.65 (m, 1H) 1.12 (d, J=10.79 Hz, 9H) 3.56-3.64 (m, 1H) 5.68-6.02 (m, 1H) 7.48 (dd, J=17.19, 5.14 Hz, 1H) 7.55-7.62 (m, 1H) 8.36 (d, J=5.27 Hz, 1H).

### Step 2—Synthesis of N((2-chloropyridin-4-yl)(cyclopropyl)methyl)-N,2-dimethylpropane-2-sulfinamide (R-3)

R-3 was made in a similar fashion to N-4 using N-((2-chloropyridin-4-yl)(cyclopropyl)methyl)-2-methylpropane-2-sulfinamide (R-2) in place of N-(2-(2-chloropyridin-4-yl)butan-2-yl)-2-methylpropane-2-sulfinamide (N-3). Scheme N, Step 3. LCMS 301.2 [M+1].

### Synthesis of N-(1-(2-bromopyridin-4-yl)-2,2-dimethylpropyl)-N,2-dimethylpropane-2-sulfinamide (S-1)

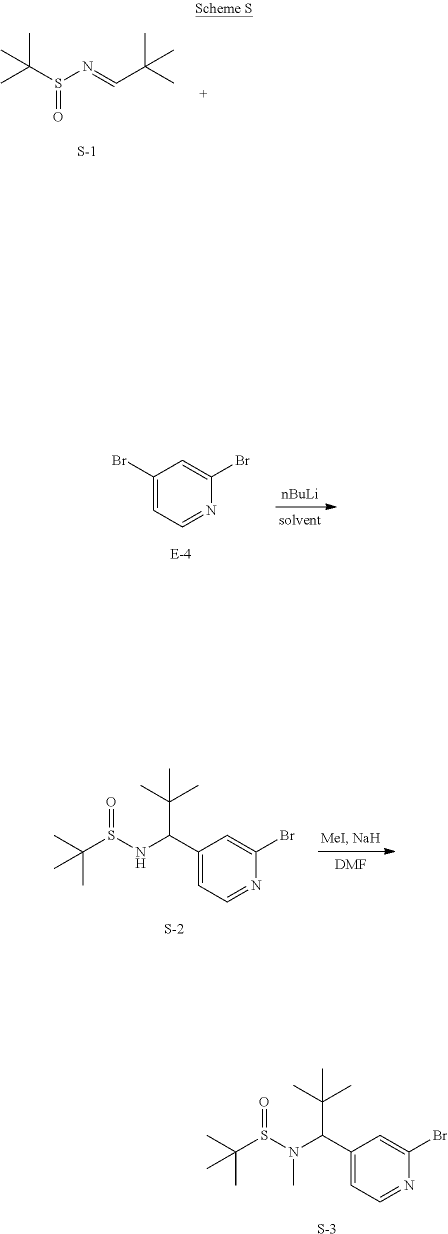

### Synthesis of N-(2,2-dimethylpropylidene)-2-methylpropane-2-sulfinamide

S-1 was made in a similar fashion to Q-1 using pivalaldehyde in place of propionaldehyde. (Scheme Q, Step 1)

### Step 1-N(1-(2-bromopyridin-4-yl)-2,2-dimethylpropyl)-2-methylpropane-2-sulfinamide

To a solution of 2,4-dibromopyridine (E-4) (2750 mg, 11.6 mmol) in MTBE (60.5 mL, 0.12 M), nBuLi (744 mg, 11.6 mmol, 2.5 M in hexanes) was added dropwise at −78° C. under nitrogen. After 10 min, S-1 (1374 mg, 7.26 mmol) in MTBE (3.63 mL) was added dropwise over 5 min. Reaction flask was removed from cold bath and warmed to room temp over 1 h. After 1 h cooled reaction to 0° C. and quenched with sat. NH₄Cl and extracted with EtOAc. Organic layer washed with water and brine, then dried over Na₂SO₄ and concentrated under reduced pressure. The crude residue was purified by column chromatography (80 g silica gel, 0-100% EtOAc/Heptane) to afford S-2 (1609 mg, 64%) as a brown solid. LCMS 348.9, 346.9 [M+1]; ¹H NMR (400 MHz, CDCl₃) δ 8.31 (dd, J=5.0, 0.7 Hz, 1H), 7.39 (dt, J=1.4, 0.6 Hz, 1H), 7.15 (dd, J=5.2, 1.5 Hz, 1H), 4.10 (d, J=1.8 Hz, 1H), 3.55 (s, 1H), 1.23 (s, 9H), 0.96 (s, 9H).

### Step 2—N-(1-(2-bromopyridin-4-yl)-2,2-dimethylpropyl)-N,2-dimethylpropane-2-sulfinamide

To a solution of S-2 (310 mg, 0.8937 mmol) in THF (8.12 mL, 0.11 M), NaH (53.6 mg, 1.34 mmol) was added at 0° C. under Nitrogen. After 30 min MeI (381 mg, 2.68 mmol, 0.167 mL) was added dropwise. Reaction flask was removed from cold bath and warmed to room temp overnight. After 18 h flask was cooled to 0° C. and quenched with water and extracted into EtOAc. Organic layer washed with brine, dried with Na₂SO₄ and concentrated under reduced pressure. Crude residue purified by column chromatography (12 g silica gel, 0-100% EtOAc/Heptane) to afford S-3 (116 mg, 36%) as a white solid. LCMS 362.9, 360.9 [M+H]; ¹H NMR (400 MHz, CDCl₃) δ 8.32 (d, J=5.1 Hz, 1H), 7.42 (d, J=1.5 Hz, 1H), 7.24 (dd, J=5.1, 1.6 Hz, 1H), 3.96 (s, 1H), 2.81 (s, 3H), 1.04 (s, 9H), 1.02 (s, 9H).

### Synthesis of N-(1-(2-chloropyridin-4-yl)-3,3,3-trifluoropropyl)-2-methylpropane-2-sulfinamide (T-2)

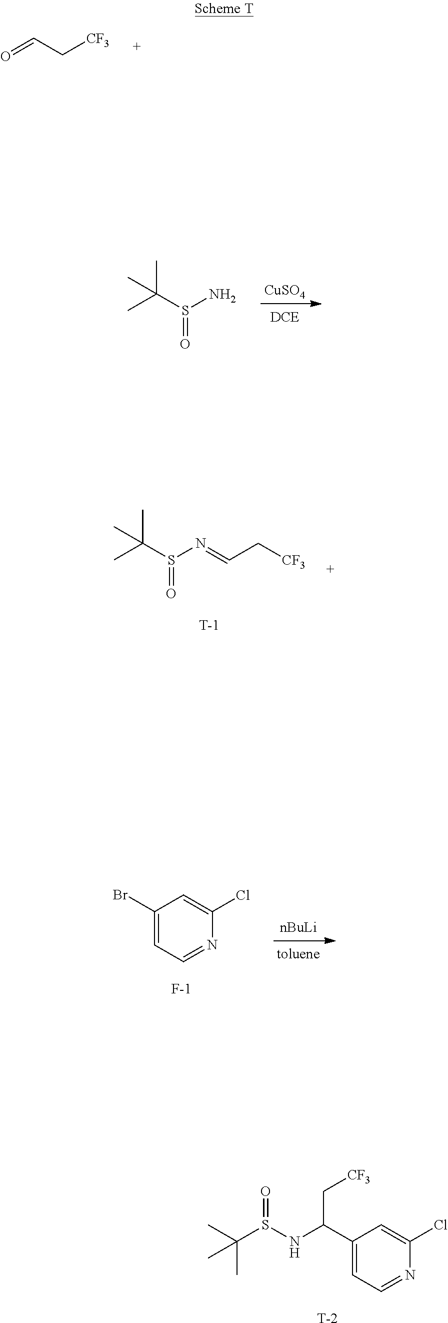

### Step 1—Synthesis of (E)-2-methyl-N-(3,3,3-trifluoropropylidene)propane-2-sulfinamide (T-1)

To a solution of 3,3,3-trifluoropropanal (4520 mg, 40.34 mmol) in DCE (100 mL) was added 2-methylpropane-2-sulfinamide (1600 mg, 13.20 mmol) and CuSO₄ (4000 mg, 25.06 mmol) at 10° C. After addition, the reaction was stirred at 70° C. for 16 h then filtered. The filter cake was rinsed with DCM (10 mL×2) and the resulting filtrate was concentrated. The crude residue was purified by column chromatography (Biotage, 0-15% EtOAc/PE) to give T-1 (1.8 g, 63%) as a brown liquid. ¹H NMR (400 MHz, DMSO-d6) δ=7.88-7.84 (m, 1H), 3.85-3.66 (m, 2H), 1.12 (s, 9H).

### Step 2—Synthesis of N(1-(2-chloropyridin-4-yl)-3,3,3-trifluoropropyl)-2-methylpropane-2-sulfinamide (T-2)

T-2 was made in a similar fashion to R-2 using (E)-2-methyl-N-(3,3,3 trifluoropropylidene)propane-2-sulfinamide (T-1) in place of (E)-N-(cyclopropylmethylene)-2-methylpropane-2-sulfinamide (R-1) Scheme R, Step 1. ¹H NMR (400 MHz, CHLOROFORM-d) δ=8.43 (d, J=5.0 Hz, 1H), 7.35 (s, 1H), 7.23 (d, J=5.3 Hz, 1H), 4.80-4.74 (m, 1H), 3.81 (br s, 1H), 2.76-2.54 (m, 2H), 1.24 (s, 9H).

### Synthesis of N-(1-(2-chloropyridin-4-yl)cyclopentyl)-N,2-dimethylpropane-2-sulfinamide (U-3)

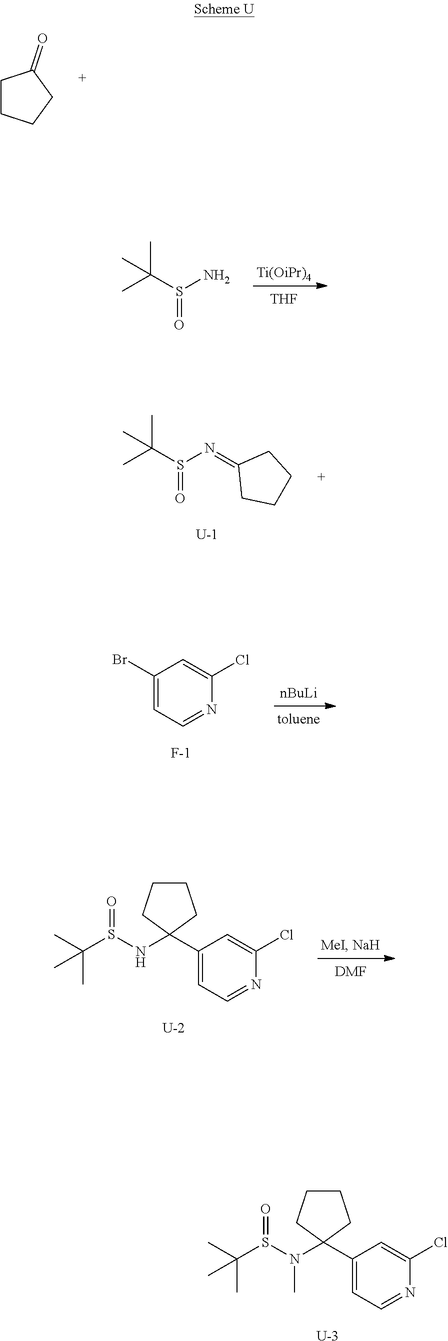

### Step 1—Synthesis of N-cyclopentylidene-2-methylpropane-2-sulfinamide (U-1)

To a solution of cyclopentanone (50 g, 594 mmol) in THF (1000 mL) was added 2-methylpropane-2-sulfinamide (72.0 g, 594 mmol) and titanium isopropoxide (422 g, 1190 mmol) at 15° C. After addition, the reaction was stirred at 75° C. for 16 h. The reaction mixture was cooled to room temperature, then poured into 1500 mL of brine and 1000 mL of EtOAc. The mixture was filtered through Celite and rinsed with EtOAc (200 mL×2). The filtrate was separated, and aqueous layer was extracted with EtOAc (800 mL). The combined extracts were washed with brine (1000 mL), then dried over Na₂SO₄ and concentrated. The crude product was purified by column chromatography (330 g silica gel, 10-30% EtOAc/PE) to give U-1 (83.5 g, 75%) as red oil. LCMS 188.3 [M+1]; ¹H NMR (400 MHz, CHLOROFORM-d) δ ppm 2.86-2.97 (m, 1H) 2.54-2.60 (m, 1H) 2.47-2.53 (m, 2H) 1.90 (tt, 2H) 1.76-1.83 (m, 2H) 1.24 (s, 9H).

### Step 2—Synthesis of N-(1-(2-chloropyridin-4-yl)cyclopentyl)-2-methylpropane-2-sulfinamide (U-2)

U-2 was made in a similar fashion to R-2 using N-cyclopentylidene-2-methylpropane-2-sulfinamide (U-1) in place of (E)-N-(cyclopropylmethylene)-2-methylpropane-2-sulfinamide (R-1) Scheme R, Step 1. LCMS 301.0 [M+1]; ¹H NMR (400 MHz, METHANOL-d4) δ ppm 8.29 (d, 1H) 7.61 (d, 1H) 7.49 (dd, 1H) 2.40-2.49 (m, 1H) 2.23-2.36 (m, 1H) 1.81-1.98 (m, 6H) 1.21 (s, 9H).

### Step 3—Synthesis of N-(1-(2-chloropyridin-4-yl)cyclopentyl)-N,2-dimethylpropane-2-sulfinamide (U-3)

U-3 was made in a similar fashion to N-4 using N-(1-(2-chloropyridin-4-yl)cyclopentyl)-2-methylpropane-2-sulfinamide (U-2) in place of N-(2-(2-chloropyridin-4-yl)butan-2-yl)-2-methylpropane-2-sulfinamide (N-3). Scheme N, Step 3. LCMS 315.0 [M+1].

### Synthesis of N-(1-(2-chloropyridin-4-yl)cyclobutyl)-N,2-dimethylpropane-2-sulfinamide (V-1)

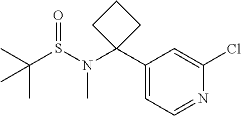 [SMILES: CN(S(=O)C(C)(C)C)C1(c2ccnc(Cl)c2)CCC1]

V-1 was made in a similar fashion to U-3 starting with cyclobutanone in place of cyclopentanone (Scheme U). LCMS 301.2 [M+1].

### Synthesis of N-(2-(2-chloropyridin-4-yl)-4,4,4-trifluorobutan-2-yl)-2-methylpropane-2-sulfinamide (W-1)

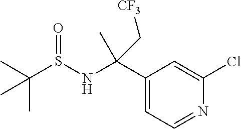 [SMILES: CC(CC(F)(F)F)(NS(=O)C(C)(C)C)c1ccnc(Cl)c1]

W-1 was made in a similar fashion to U-2 using 4,4,4-trifluorobutan-2-one in place of cyclopentanone (Scheme U, Step 1) and (E)-2-methyl-N-(4,4,4-trifluorobutan-2-ylidene)propane-2-sulfinamide in place of N-cyclopentylidene-2-methylpropane-2-sulfinamide (U-1) Scheme U, Step 2. LCMS 343.2 [M+1]; ¹H NMR (400 MHz, CHLOROFORM-d) δ 8.40-8.49 (m, 1H), 7.49 (s, 1H), 7.41 (dd. J=1.69, 5.32 Hz, 1H), 3.87 (s, 1H), 2.86-3.02 (m, 1H), 2.69-2.82 (m, 1H), 1.29 (s, 9H), 1.25 (s, 3H).

### Synthesis of N-(2-(2-chloropyridin-4-yl)-1,1,1-trifluoropropan-2-yl)-2-methylpropane-2-sulfinamide (X-1)

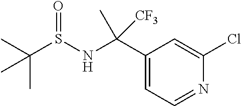 [SMILES: CC(C)(C)S(=O)NC(C)(c1ccnc(Cl)c1)C(F)(F)F]

X-1 was made in a similar fashion to U-2 using 1,1,1-trifluoropropan-2-one in place of cyclopentanone (Scheme U, Step 1) and (E)-2-methyl-N-(1,1,1-trifluoropropan-2-ylidene)propane-2-sulfinamide in place of N-cyclopentylidene-2-methylpropane-2-sulfinamide (U-1) Scheme U, Step 2. LCMS 329.1 [M+1].

### Synthesis of N-(1-(2-chloropyridin-4-yl)-1-cyclopropylethyl)-N,2-dimethylpropane-2-sulfinamide (Y-1)

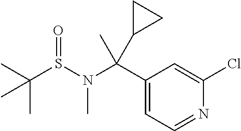 [SMILES: CN(S(=O)C(C)(C)C)C(C)(c1ccnc(Cl)c1)C1CC1]

Y-1 was made in a similar fashion to U-3 starting with 1-cyclopropylethan-1-one in place of cyclopentanone (Scheme U). LCMS 315.1 [M+1].

### Synthesis of N-(2-(2-chloropyridin-4-yl)-3-methylbutan-2-yl)-N,2-dimethylpropane-2-sulfinamide (Z-1)

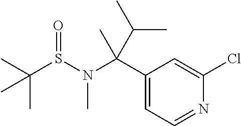 [SMILES: CC(C)C(C)(c1ccnc(Cl)c1)N(C)S(=O)C(C)(C)C]

Z-1 was made in a similar fashion to U-3 starting with 3-methylbutan-2-one in place of cyclopentanone (Scheme U). LCMS 317.0 [M+1].

### Synthesis of (S)—N-(2-(2-bromopyridin-4-yl)-1-methoxypropan-2-yl)-N,2-dimethylpropane-2-sulfinamide (AA-7)

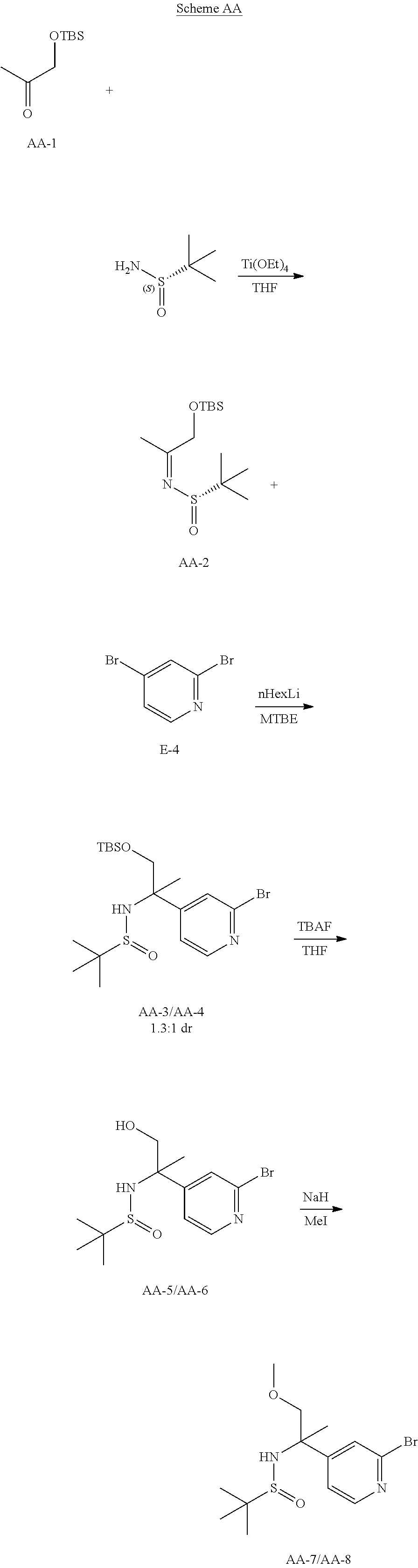

### Step 1—Synthesis of (S,Z)—N-(1-((tert-butyldimethylsilyl)oxy)propan-2-ylidene)-2-methylpropane-2-sulfinamide (AA-2)

To a solution of AA-1 (2000 mg, 10.62 mmol, 2.05 mL) and (S)-2-methylpropane-2-sulfinamide (1290 mg, 10.6 mmol) in THF (26.5 mL, c=0.4 M) was added Ti(OEt)₄ (6060 mg, 26.5 mmol), and the mixture was stirred at 70° C. for 20 h. Then the reaction mixture was cooled to room temperature, and it was poured into equal volume of brine. The mixture was filtered through a short pad of celite with ethyl acetate as eluent. The organic layer was separated, and the aqueous phase was extracted three times with EtOAc. The combined organic layers were washed with brine, then dried over Na2SO4, filtered and concentrated. The residue was purified by column chromatography (0-40% EA/heptane, 40 g silica gel column) on Teledyne ISCO to give AA-2 (1003 mg, LIQUID-OIL, 32%). LCMS 292.1 [M+1]; ¹H NMR (400 MHz, Chloroform-d) δ 4.23 (s, 2H), 2.33 (s, 3H), 1.24 (s, 9H), 0.91 (s, 9H), 0.08 (s, 6H).

### Step 2—Synthesis of (S)—N-(2-(2-bromopyridin-4-yl)-1-((tert-butyldimethylsilyl)oxy)propan-2-yl)-2-methylpropane-2-sulfinamide (diastereomer 1: AA-3, diastereomer 2: AA-4)

To a solution of E-4 (780 mg, 3.29 mmol) in MTBE (10.3 mL, c=0.2 M) was added n-hexyl lithium (303 mg, 3.29 mmol, 1.29 mL, 2.56 M) dropwise at −78° C. After stirring at −78° C. for 10 min, a solution of AA-2 (600.0 mg, 2.06 mmol) in MTBE (0.5 mL) was added dropwise. The reaction turned dark solution and stirred at the same temperature for 30 min. The reaction was quenched by adding sat. NH₄Cl. The mixture was extracted three times with EtOAc. The combined organic layers were washed with brine, then dried over Na2SO4, filtered, and concentrated. The residue was purified by column chromatography (0-80% EA/heptane, 4 g silica gel column) on Teledyne ISCO to give AA-3 (53 mg, LIQUID-OIL, 29%) and AA-4 (34 mg, LIQUID-OIL, 18%). AA-3: LCMS 449.1, 451.1 [M+1]; ¹H NMR (400 MHz, Chloroform-d) δ 8.31 (dd, J=5.3, 0.7 Hz, 1H), 7.61 (dd, J=1.7, 0.7 Hz, 1H), 7.44 (dd, J=5.3, 1.7 Hz, 1H), 4.24 (s, 1H), 3.74 (d, J=1.5 Hz, 2H), 1.57 (s, 3H), 1.24 (s, 9H), 0.82 (s, 9H), −0.01 (s, 3H), −0.08 (s, 3H). AA-4: LCMS 449.1, 451.1 [M+1]; ¹H NMR (400 MHz, Chloroform-d) δ 8.31 (dd, J=5.3, 0.6 Hz, 1H), 7.54 (dd, J=1.7, 0.7 Hz, 1H), 7.29 (dd, J=5.3, 1.7 Hz, 1H), 4.29 (s, 1H), 3.77 (d, J=9.7 Hz, 1H), 3.65 (d, J=9.7 Hz, 1H), 1.70 (s, 3H), 1.25 (s, 9H), 0.85 (s, 9H), 0.03 (d, J=11.7 Hz, 6H).

### Step 3—Synthesis of (S)—N-(2-(2-bromopyridin-4-yl)-1-hydroxypropan-2-yl)-2-methylpropane-2-sulfinamide (AA-5)

To a solution of AA-3 (206.0 mg, 0.458 mmol) in THF (2.29 mL, c=0.2 M) was added TBAF (240 mg, 0.917 mmol, 0.917 mL, 1.0 M). The mixture was stirred at room temperature overnight. The reaction mixture was concentrated and loaded onto 4 g silica gel column directly and purified on Teledyne ISCO with 0-100% EtOAc (containing 2% HOAc, v/v)/heptane to give product AA-5 (163 mg, LIQUID-OIL, 106%) which contains some HOAc. LCMS 335.0, 337.0 [M+1]; ¹H NMR (400 MHz, Chloroform-d) δ 8.30 (dd, J=5.3, 0.6 Hz, 1H), 7.55 (dd, J=1.7, 0.6 Hz, 1H), 7.31 (dd, J=5.3, 1.7 Hz, 1H), 4.21 (s, 1H), 4.10 (d, J=12.1 Hz, 1H), 3.84 (d, J=12.0 Hz, 1H), 1.47 (s, 3H), 1.26 (s, 9H).

### Step 4—Synthesis of (S)—N-(2-(2-bromopyridin-4-yl)-1-methoxypropan-2-yl)-2-methylpropane-2-sulfinamide (AA-7)

To a solution of AA-5 (163.0 mg, 0.486 mmol) in DMF (4.86 mL, c=0.1 M) was added NaH (97.2 mg, 2.43 mmol) slowly at 0° C., stirred at the same temperature for 0.5 h, then MeI (276 mg, 1.94 mmol, 121 uL) was added. Then the mixture was stirred at room temperature for 2 hours before quenched with sat·NH₄Cl (~5 mL). The mixture was extracted with EtOAc (5 mL*2). The organic layer was combined, washed with brine (5 mL*2) and dried over Na₂SO4. The organic solvent was filtered and concentrated. The residue purified on Teledyne ISCO (4 g silica gel, 0-80% EtOAc/heptane) to give AA-7 (115.0 mg, LIQUID-OIL, 65.1%). LCMS 363.0, 365.0 [M+1]; ¹H NMR (400 MHz, Chloroform-d) δ 8.31 (dd, J=5.3, 0.6 Hz, 1H), 7.49 (dd, J=1.7, 0.6 Hz, 1H), 7.34 (dd, J=5.3, 1.7 Hz, 1H), 3.75 (d, J=9.6 Hz, 1H), 3.57 (d, J=9.5 Hz, 1H), 3.27 (s, 3H), 2.35 (s, 3H), 1.60 (s, 3H), 1.27 (s, 9H).

### Synthesis of (S)—N-(2-(2-bromopyridin-4-yl)-1-methoxypropan-2-yl)-N,2-dimethylpropane-2-sulfinamide (AA-8)

AA-8 was made in a similar fashion to AA-7 using AA-4 in place of AA-3 (Scheme AA: Step 3) and AA-6 in place of AA-5 (Scheme AA: Step 4). AA-8: LCMS 363.0, 365.0 [M+1]; ¹H NMR (400 MHz, Chloroform-d) δ 8.32 (dd, J=5.2, 0.7 Hz, 1H), 7.54 (dd, J=1.7, 0.7 Hz, 1H), 7.40 (dd, J=5.3, 1.7 Hz, 1H), 3.70 (d, J=10.0 Hz, 1H), 3.60 (d, J=10.0 Hz, 1H), 3.32 (s, 3H), 2.36 (s, 3H), 1.60 (s, 3H), 1.27 (s, 9H).

### Synthesis of N-(3-(6-bromopyrimidin-4-yl)pentan-3-yl)-2-methylpropane-2-sulfinamide (AB-2)

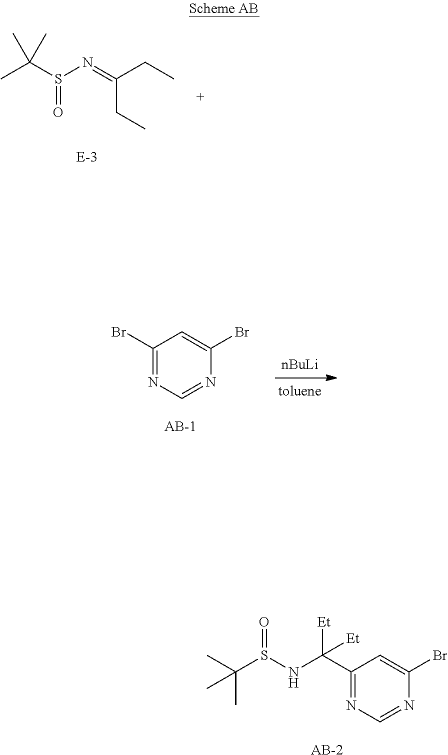

AB-2 was made in a similar fashion to R-2 using 4,6-dibromopyrimidine (AB-1) and 2-methyl-N-(pentan-3-ylidene)propane-2-sulfinamide (E-3) in place of 4-bromo-2-chloropyridine (F-1) and (E)-N-(cyclopropylmethylene)-2-methylpropane-2-sulfinamide (R-1) Scheme R, Step 1. LCMS 350.1, 348.1 [M+1]; ¹H NMR (400 MHz, CHLOROFORM-d) δ=8.90 (s, 1H), 7.55 (d, J=0.6 Hz, 1H), 4.87 (s, 1H), 2.25-2.12 (m, 2H), 2.05-1.97 (m, 2H), 1.29 (s, 9H), 0.74 (t, J=7.3 Hz, 3H), 0.67 (t, J=7.3 Hz, 3H).

### EXAMPLES

### Example 1

### Synthesis of N-(4-(3-aminopentan-3-yl)pyridin-2-yl)-3-(1-methyl-1H-1,2,3-triazol-5-yl)-2,7-naphthyridine-1,6-diamine (1)

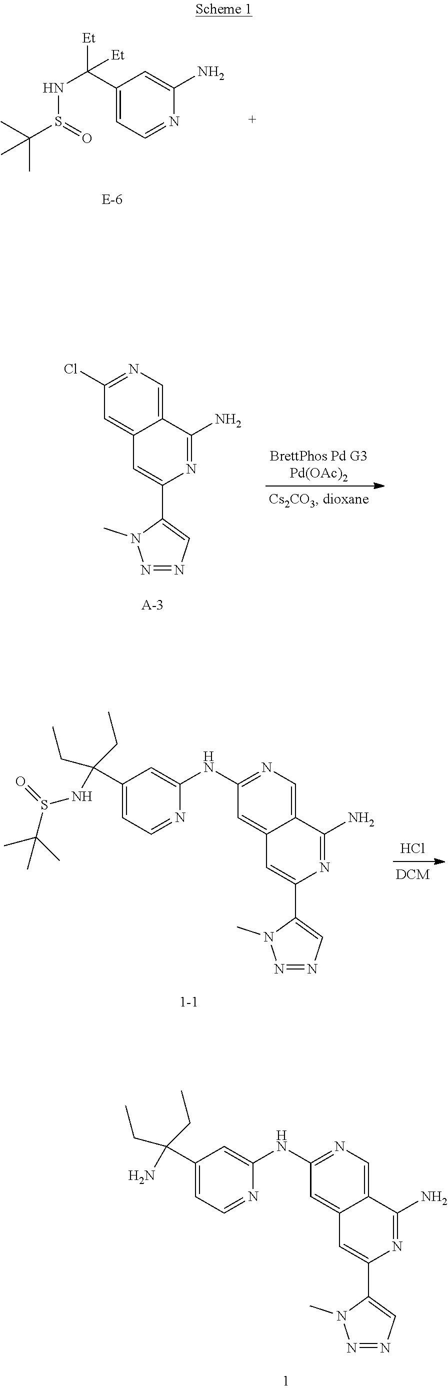

### Step 1—Synthesis of N-(3-(2-((8-amino-6-(1-methyl-1H-1,2,3-triazol-5-yl)-2,7-naphthyridin-3-yl)amino)pyridin-4-yl)pentan-3-yl)-2-methylpropane-2-sulfinamide (1-1)

A mixture of A-3 (15900 mg, 60.9 mmol), E-6 (15000 mg, 52.92 mmol), Cs₂CO₃ (37900 mg, 116.0 mmol), BrettPhos Pd G3 (959.0 mg, 1.06 mmol) in 1,4-dioxane (150 mL) was degassed with N₂ four times. The reaction was stirred at 100° C. for 64 h then diluted with EtOAc (500 mL) and washed with water (500 mL). The aqueous phase was extracted with EtOAc (3×250 mL). The combined organic layers were washed with brine, then dried (Na₂SO₄), filtered and concentrated. The crude residue was purified by column chromatography (120 g silica gel, 0-5% MeOH/DCM) to give 1-1 (18.3 g, 64%) as an orange solid. LCMS 508.3 [M+1]; ¹H NMR (400 MHz, METHANOL-d4) δ=9.19 (s, 1H), 8.25 (d, J=5.5 Hz, 1H), 8.17 (s, 1H), 8.07 (s, 1H), 7.46 (d, J=1.0 Hz, 1H), 7.22 (s, 1H), 7.00 (dd, J=1.6, 5.6 Hz, 1H), 5.49 (s, 1H), 5.04 (s, 1H), 4.40 (s, 3H), 3.35 (s, 3H), 2.22-2.11 (m, 2H), 2.02 (dt, J=7.3, 13.5 Hz, 2H), 1.32 (s, 9H), 0.85-0.79 (m, 6H).

### Step 2—Synthesis of N-(4-(3-aminopentan-3-yl)pyridin-2-yl)-3(1-methyl-1H-1,2,3-triazol-5-yl)-2,7-naphthyridine-1,6-diamine (1)

To a solution of 1-1 (18300 mg, 36.048 mmol) in DCM (300 mL) was added 4 M HCl in 1,4-dioxane (690 mg, 19 mmol). The reaction was stirred at 25° C. for 16 h then filtered and rinsed with DCM. The resulting solid was dried in the vacuum oven to give 1 (12700 mg, 50%) as a yellow solid as the HCl salt. LCMS 404.3 [M+1]; ¹H NMR (400 MHz, DMSO-d6) δ=11.35 (br s, 1H), 9.64 (s, 1H), 8.92 (br s, 3H), 8.45 (d, J=5.8 Hz, 1H), 8.32 (s, 1H), 8.18 (br s, 1H), 7.52 (s, 1H), 7.47 (s, 1H), 7.30 (d, J=5.0 Hz, 1H), 4.37-4.25 (m, 3H), 2.01 (tt, J=7.2, 14.7 Hz, 4H), 0.84 (t, J=7.4 Hz, 6H).

### Example 2

### Synthesis of 3(1-amino-6-((4-(3-aminopentan-3-yl)pyridin-2-yl)amino)-2,7-naphthyridin-3-yl)oxazolidin-2-one (2)

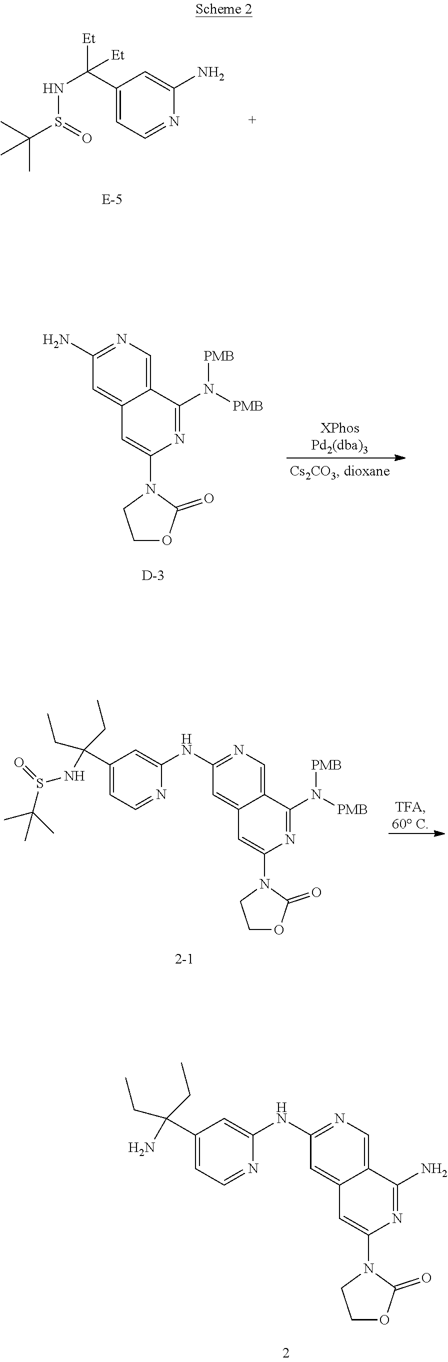

### Step 1—Synthesis of N(3-(2-((8-(bis(4-methoxybenzyl)amino)-6-(2-oxooxazolidin-3-yl)-2,7-naphthyridin-3-yl)amino)pyridin-4-yl)pentan-3-yl)-2-methylpropane-2-sulfinamide (2-1)

A mixture of D-3 (1340.0 mg, 2.760 mmol), E-5 (1250 mg, 3.59 mmol), Cs₂CO₃ (1800 mg, 5.52 mmol), X-Phos (263 mg, 0.552 mmol) and Pd₂(dba)₃ (253 mg, 0.276 mmol) in anhydrous 1,4-dioxane (30.0 mL) was degassed with N₂ for 6 min then stirred at 100° C. for 15 h. The reaction was filtered through pad of Celite and rinsed with EtOAc (10 mL×4). The filtrate was concentrated and purified by column chromatography (40 g silica gel, 50-100% EtOAc/PE) to give 2-1 (1453 mg, 70%) as a yellow solid. LCMS 752.3 [M+1].

### Step 2—Synthesis of Synthesis of 3-(1-amino-6-((4-(3-aminopentan-3-yl)pyridin-2-yl)amino)-2,7-naphthyridin-3-yl)oxazolidin-2-one (2)

A solution of 2-1 (1600 mg, 2.128 mmol) in TFA (30 mL) was stirred at 80° C. for 32 h. The reaction was concentrated to remove most of the TFA, then diluted with MeOH (5 mL) and DCM (15 mL). The mixture was basified to pH=10 with NH₄OH then concentrated. The crude residue was purified by column chromatography (20 silica gel, 50-100% EtOAc/PE then 0-10% MeOH/DCM) to give a crude product (2.5 g, 71.22% of purity by LCMS) as yellow oil. The yellow oil was further purified by prep HPLC to give 2 (507 mg, 58%) as a white solid. LCMS 408.3 [M+1]; ¹H NMR (400 MHz, DMSO-d6) δ ppm 9.61 (s, 1H) 9.17 (s, 1H) 8.22 (d, 1H) 8.20 (s, 1H) 7.36 (s, 1H) 7.31 (s, 1H) 7.20 (br s, 2H) 6.94 (dd, 1H) 4.38-4.45 (m, 2H) 4.15-4.22 (m, 2H) 1.66-1.75 (m, 2H) 1.51-1.64 (m, 4H) 0.66 (t, 6H).

### Example 3 and Example 4

### Synthesis of enantiomers N-(5-ethyl-5-(methylamino)-6,7-dihydro-5H-cyclopenta[c]pyridin-1-yl)-3-(1-methyl-1H-1,2,3-triazol-5-yl)-2,7-naphthyridine-1,6-diamine (3,4)

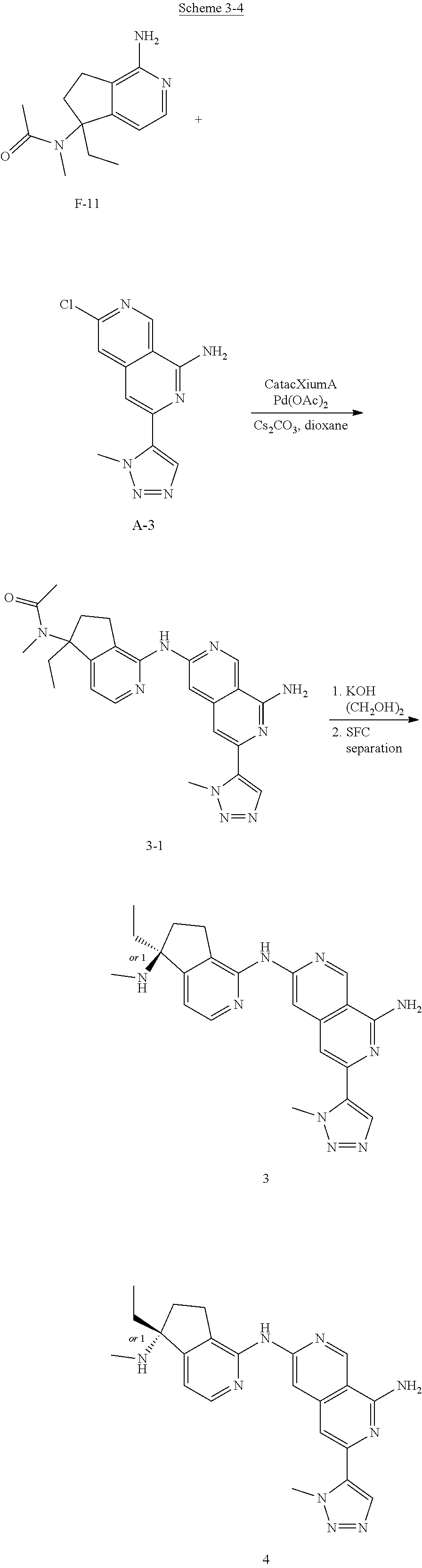

### Step 1—Synthesis of N-(1-((8-amino-6-(1-methyl-1H-1,2,3-triazol-5-yl)-2,7-naphthyridin-3-yl)amino)-5-ethyl-6,7-dihydro-5H-cyclopenta[c]pyridin-5-yl)-N-methylacetamide (3-1)

3-1 was made in a similar fashion to 1-1 using racemic F-11 in place of E-6 and CatacXiumA in place of BrettPhos Pd G3 (Scheme 1), 68% yield as yellow oil. LCMS 458.2 [M+1].

### Step 2—Synthesis of enantiomers N⁶-(5-ethyl-5-(methylamino)-6,7-dihydro-5H-cyclopenta[c]pyridin-1-yl)-3-(1-methyl-1H-1,2,3-triazol-5-yl)-2,7-naphthyridine-1,6-diamine (3,4)

To a solution of 3-1 (233 mg, 0.509 mmol) in ethylene glycol (5 mL) was added KOH (200 mg, 3.56 mmol). The reaction was stirred at 135° C. for 14 h. Additional KOH (200 mg, 3.56 mmol) was added and reaction was stirred at 135° C. for 14 h. More KOH (200 mg, 3.56 mmol) was added, and the reaction was heated to 160° C. for 4 h, then at 135° C. for 14 h. The reaction was concentrated to dryness. The crude residue was purified by column chromatography (12 g silica gel, 0-25% MeOH/DCM) then further purified by prep HPLC to give 15 mg as a racemic mixture. The enantiomers were separated by Prep SFC to give enantiomer 1: 3 (5.3 mg, 3%) as a white solid. LCMS 416.4 [M+1]; ¹H NMR (400 MHz, METHANOL-d4) δ ppm 9.21 (s, 1H) 8.47 (s, 1H) 8.26 (d, J=5.14 Hz, 1H) 8.09 (s, 1H) 7.27 (s, 1H) 6.95 (d, J=5.26 Hz, 1H) 4.42 (s, 3H) 2.90-3.10 (m, 2H) 2.25-2.32 (m, 5H) 1.82-1.98 (m, 2H) 0.87 (t, J=7.46 Hz, 3H): [α]25D−64.6° (c=0.10 g/100 mL, methanol) and enantiomer 2: 4 (3.6 mg, 2%) as a white solid. LCMS 416.4 [M+1]; ¹H NMR (400 MHz, METHANOL-d4) δ ppm 9.19 (s, 1H) 8.44 (s, 1H) 8.23 (d, J=5.14 Hz, 1H) 8.09 (s, 1H) 7.25 (s, 1H) 6.93 (d, J=5.14 Hz, 1H) 4.41 (s, 3H) 2.86-3.05 (m, 2H) 2.18-2.28 (m, 5H) 1.77-1.92 (m, 2H) 0.85 (t, J=7.46 Hz, 3H); [α]25D+19.2° (c=0.10 g/100 mL, methanol).

### Example 5 and Example 6

### Synthesis of enantiomers N⁶-(5-ethyl-5-(methylamino)-6,7-dihydro-5H-cyclopenta[c]pyridin-3-yl)-3-(1-methyl-1H-1,2,3-triazol-5-yl)-2,7-naphthyridine-1,6-diamine (5,6)

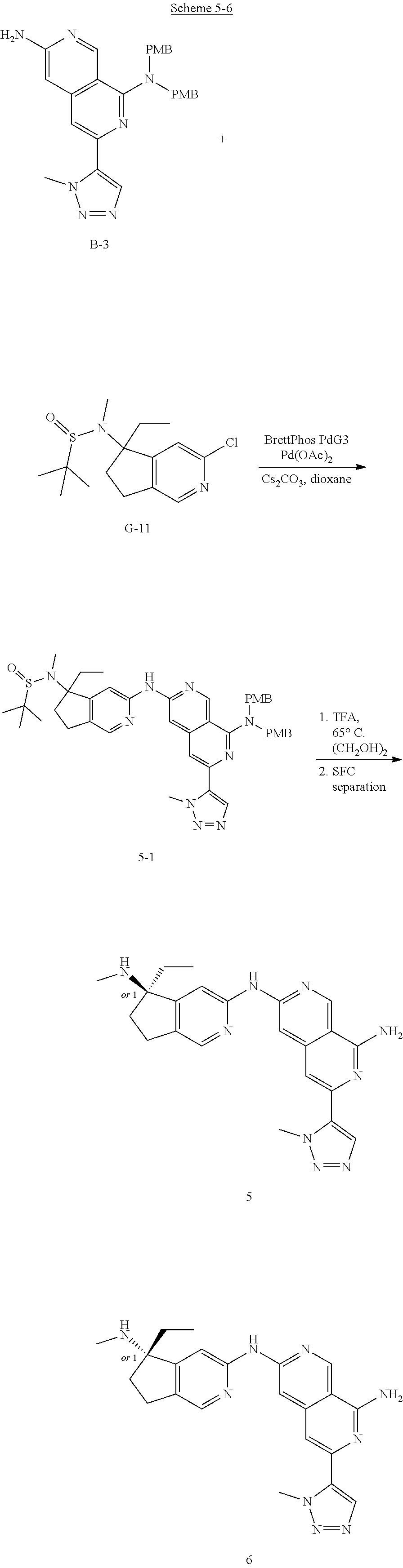

### Step 1—Synthesis of N(3-((8-(bis(4-methoxybenzyl)amino)-6-(1-methyl-1H-1,2,3-triazol-5-yl)-2,7-naphthyridin-3-yl)amino)-5-ethyl-6,7-dihydro-5H-cyclopenta[c]pyridin-5-yl)-N,2-dimethylpropane-2-sulfinamide (5-1)

To a solution of G-11 (520 mg, 1.65 mmol) in 1,4-dioxane (15 m) was added B-3 (875 mg, 1.82 mmol), Cs₂CO₃ (1350 mg, 4.13 mmol) and BrettPhos Pd G3 (89.8 mg, 0.0991 mmol) at 20° C. The mixture was evacuated and back filled with N₂ 6 times then heated to 100° C. for 15 h. The reaction was diluted with DCM (30 mL) and MeOH (10 mL) then filtered and rinsed with DCM. The filtrate was concentrated and purified by column chromatography (24 g silica gel, 10-100% EtOAc/PE) to give 5-1 (800 mg, 64%) as a yellow solid. LCMS 760.5 [M+1].

### Step 2—Synthesis of enantiomers N⁶-(5-ethyl-5-(methylamino)-6,7-dihydro-5H-cyclopenta[c]pyridin-3-yl)-3-(1-methyl-1H-1,2,3-triazol-5-yl)-2,7-naphthyridine-1,6-diamine (5, 6)

A solution of 5-1 (700.0 mg, 0.921 mmol) in TFA (30.0 mL) was heated to 65° C. for 2.5 h. The residue was diluted with DCM (20 mL) and MeOH (3 mL) then basified (pH=9) with NH₄OH. The mixture was concentrated and purified by column chromatography (12 g silica gel, 6-17% MeOH/EtOAc) then further purified by prep HPLC to give 280 mg as a racemic mixture. The enantiomers were separated by Prep SFC to give enantiomer 1: 5 (68.5 mg, 18%) as a brown solid. LCMS 416.3 [M+1]; ¹H NMR (400 MHz, DMSO-d6) δ ppm 9.79 (s, 1H) 9.29 (s, 1H) 8.22 (s, 2H) 8.14 (s, 1H) 7.31 (s, 2H) 7.23 (s, 1H) 7.17 (s, 1H) 4.38 (s, 3H) 2.85 (br dd, 1H) 2.72-2.79 (m, 1H) 2.08 (s, 3H) 2.00-2.07 (m, 1H) 1.88-1.96 (m, 1H) 1.66-1.75 (m, 1H) 1.50-1.62 (m, 1H) 0.82 (t, 3H); [α]25D=+6.06 (c=0.11 g/100 mL, MeOH) and enantiomer 2: 6 (72.9 mg, 19%) as a white solid. LCMS 416.3 [M+1]; 1H NMR (400 MHz, DMSO-d6) δ ppm 9.78 (s, 1H) 9.28 (s, 1H) 8.22 (s, 2H) 8.13 (s, 1H) 7.31 (s, 2H) 7.23 (s, 1H) 7.16 (s, 1H) 4.38 (s, 3H) 2.84 (br dd, 1H) 2.70-2.79 (m, 1H) 2.08 (s, 3H) 2.01-2.06 (m, 1H) 1.92 (ddd, 1H) 1.64-1.73 (m, 1H) 1.53-1.61 (m, 1H) 0.82 (t, 3H); [α]25D=−2.22° (c=0.09 g/100 mL, MeOH).

Examples 7-35 were made in a similar fashion to Example 5 using the appropriate chloro- or bromo-pyridine in Step 1.

| Example No./ | | MS | |
|---|---|---|---|
| Intermediates | Structure/Name | [M + 1] | NMR |
| 7 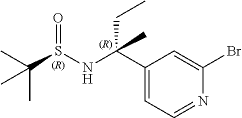 H-5 | 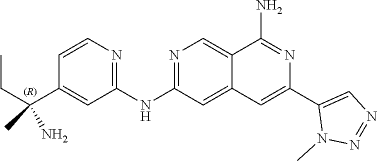 (R)-N⁶-(4-(2-aminobutan-2- yl)pyridin-2-yl)-3-(1-methyl- 1H-1,2,3-triazol-5-yl)-2,7- naphthyridine-1,6-diamine | 390.3 | ¹H NMR (400 MHz, METHANOL- d4) δ 9.21 (s, 1H), 8.24 (s, 1H), 8.25 (d, J = 5.92 Hz, 1H), 8.10 (s, 1H), 7.32 (s, 1H), 7.24 (s, 1H), 7.04 (d, J = 5.39 Hz, 1H), 4.42 (s, 3H), 1.74-1.92 (m, 2H), 1.48 (s, 3H), 0.80 (t, J = 7.40 Hz, 3H) |
| 8 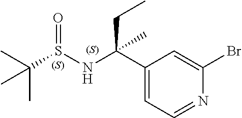 I-1 | 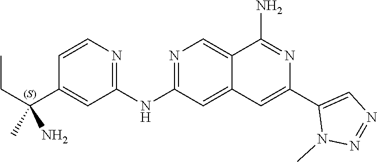 (S)-N⁶-(4-(2-aminobutan-2- yl)pyridin-2-yl)-3-(1-methyl- 1H-1,2,3-triazol-5-yl)-2,7- naphthyridine-1,6-diamine | 390.3 | ¹H NMR (400 MHz, DMSO-d6) δ = 9.82 (s, 1H), 9.31 (s, 1H), 8.36 (s, 1H), 8.25- 8.18 (m, 2H), 7.42 (s, 1H), 7.35 (br s, 2H). 7.27 (s, 1H), 7.02 (br d, J = 5.2 Hz, 1H), 4.40 (s, 3H), 1.72- 1.57 (m, 2H), 1.32 (s, 3H), 0.69 (t, J = 7.3 Hz, 3H) |
| 9 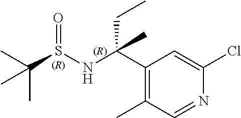 L-4 | 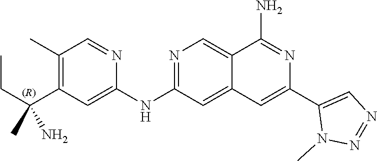 (R)-N⁶-(4-(2-aminobutan-2- yl)-5-methylpyridin-2-yl)-3-(1- methyl-1H-1,2,3-triazol-5-yl)- 2,7-naphthyridine-1,6- diamine | 404.3 | ¹H NMR (400 MHz, METHANOL- d4) δ = 9.20 (s, 1H), 8.17 (s, 1H), 8.10 (s, 1H), 8.05 (s, 1H), 7.36 (s, 1H), 7.24 (s, 1H), 4.42 (s, 3H), 2.48 (s, 3H), 2.13- 2.02 (m, 1H), 1.87 (qd, J = 7.3, 14.5 Hz, 1H), 1.57 (s, 3H), 0.80 (t, J = 7.4 Hz, 3H) |
| 10 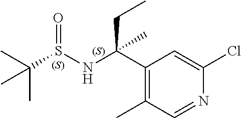 M-1 | 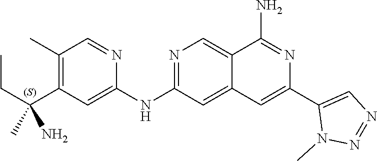 (S)-N⁶-(4-(2-aminobutan-2- yl)-5-methylpyridin-2-yl)-3-(1- methyl-1H-1,2,3-triazol-5-yl)- 2,7-naphthyridine-1,6- diamine | 404.2 | ¹H NMR (400 MHz, METHANOL- d4) δ 9.31 (s, 1H), 8.22 (s, 1H), 8.07-8.16 (m, 2H), 7.29 (s, 2H), 4.42 (s, 3H), 2.51 (s, 3H), 2.31 (br dd, J = 8.03, 15.31 Hz, 1H), 2.14 (dd, J = 7.28, 14.81 Hz, 1H), 1.81-1.86 (m, 3H), 0.94 (t, J = 7.53 Hz, 3H |
| 11 Enantiomer 1 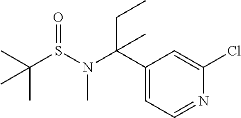 [SMILES: CCC(C)(c1ccnc(Cl)c1)N(C)S(=O)C(C)(C)C] N-4 | 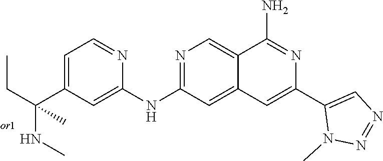 [SMILES: CC[C@](C)(NC)c1ccnc(Nc2cc3cc(-c4cnnn4C)nc(N)c3cn2)c1] 3-(1-methyl-1H-1,2,3-triazol- 5-yl)-N⁶-(4-(2- (methylamino)butan-2- yl)pyridin-2-yl)-2,7- naphthyridine-1,6-diamine | 404.3 | ¹H NMR (600 MHz, DMSO-17mm) δ = 9.79 (s, 1H), 9.30 (s, 1H), 8.33 (s, 1H), 8.23 (d, J = 5.5 Hz, 1H), 8.21 (s, 1H), 7.35 (s, 1H), 7.31 (s, 2H), 7.25 (s, 1H), 6.99 (dd, J = 1.5, 5.5 Hz, 1H), 4.38 (s, 3H), 2.05 (s, 3H), 1.71- 1.58 (m, 2H), 1.31 (s, 3H), 0.71 (t, J = 7.4 Hz, 3H); [a]D22 = −3.1° (C = 0.1 g/100 mL, MeOH) |
| 12 Enantiomer 2 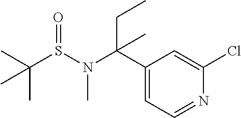 [SMILES: CCC(C)(c1ccnc(Cl)c1)N(C)S(=O)C(C)(C)C] N-4 | 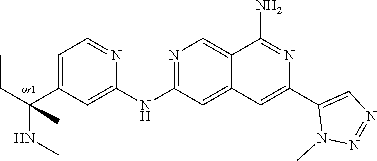 [SMILES: CC[C@@](C)(NC)c1ccnc(Nc2cc3cc(-c4cnnn4C)nc(N)c3cn2)c1] 3-(1-methyl-1H-1,2,3-triazol- 5-y1)-N⁶-(4-(2- (methylamino)butan-2- yl)pyridin-2-yl)-2,7- naphthyridine-1,6-diamine | 404.3 | ¹H NMR (600 MHz, DMSO-17mm) δ = 9.79 (s, 1H), 9.30 (s, 1H), 8.33 (s, 1H), 8.23 (d, J = 5.5 Hz, 1H), 8.21 (s, 1H), 7.35 (s, 1H), 7.31 (s, 2H), 7.25 (s, 1H), 6.99 (dd, J = 1.5, 5.3 Hz, 1H), 4.38 (s, 3H), 2.04 (s, 3H), 1.71- 1.58 (m, 2H), 1.31 (s, 3H), 0.70 (t, J = 7.4 Hz, 3H); [a]D22 = +3.2° (C = 0.1 g/100mL, MeOH) |
| 13 Enantiomer 1 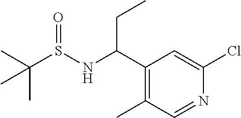 [SMILES: CCC(NS(=O)C(C)(C)C)c1cc(Cl)ncc1C] Q-2 | 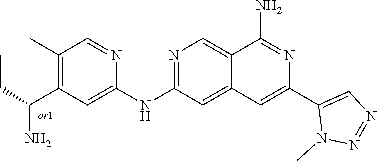 [SMILES: CC[C@@H](N)c1cc(Nc2cc3cc(-c4cnnn4C)nc(N)c3cn2)ncc1C] N⁶-(4-(1-aminopropyl)-5- methylpyridin-2-yl)-3-(1- methyl-1H-1,2,3-triazol-5-yl)- 2,7-naphthyridine-1,6- diamine | 390.3 | 1H NMR (400 MHz, METHANOL- d4) δ = 9.23- 9.17 (m, 1H), 8.23- 8.00 (m, 3H), 7.33-7.18 (m, 2H), 4.41 (s, 3H), 4.10-4.03 (m, 1H), 2.31 (s, 3H), 1.84- 1.66 (m, 2H), 0.97 (t, J = 7.4 Hz, 3H) |
| 14 Enantiomer 2 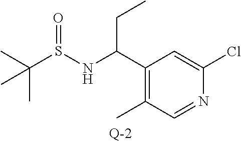 | 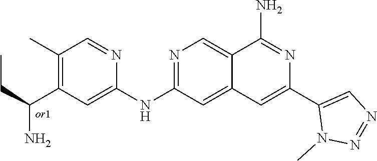 [SMILES: CC[C@H](N)c1cc(Nc2cc3cc(-c4cnnn4C)nc(N)c3cn2)ncc1C] N⁶-(4-(1-aminopropyl)-5- methylpyridin-2-yl)-3-(1- methyl-1H-1,2,3-triazol-5-yl)- 2,7-naphthyridine-1,6- diamine | 390.3 | ¹H NMR (400 MHz, METHANOL- d4) δ = 9.17 (s, 1H), 8.12- 8.03 (m, 3H), 7.29- 7.16 (m, 2H), 4.40 (s, 3H), 4.05 (t, J = 6.6 Hz, 1H), 2.36-2.25 (m, 3H), 1.81- 1.68 (m, 2H), 1.01- 0.92 (m, 3H) |
| 15 Enantiomer 1 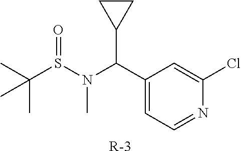 | 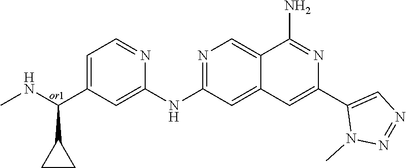 [SMILES: CN[C@@H](c1ccnc(Nc2cc3cc(-c4cnnn4C)nc(N)c3cn2)c1)C1CC1] N⁶-(4- (cyclopropyl(methylamino) methyl)pyridin-2-yl)-3-(1- methyl-1H-1,2,3-triazol-5-yl)- 2,7-naphthyridine-1,6- diamine | 402.3 | 1H NMR (400 MHz, DMSO-d6) δ ppm 0.19- 0.27 (m, 1 H) 0.30-0.43 (m, 2 H) 0.50- 0.60 (m, 1 H) 0.87-0.98 (m, 1 H) 2.17 (s, 3 H) 2.66 (d, J = 8.53 Hz, 1H) 4.39 (s, 3 H) 6.93 (d, J = 5.27 Hz, 1 H) 7.26 (s, 1 H) 7.31 (s, 1 H) 7.35 (s, 2 H) 8.21- 8.24 (m, 2 H) 8.32 (s, 1 H) 9.30 (s, 1 H) 9.89 (s, 1 H); [α]24D = +19.3° (C = 1.0 mg/mL, MeOH) |
| 16 Enantiomer 2 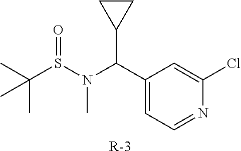 | 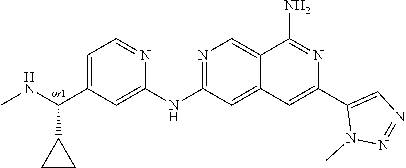 [SMILES: CN[C@H](c1ccnc(Nc2cc3cc(-c4cnnn4C)nc(N)c3cn2)c1)C1CC1] N⁶-(4- (cyclopropyl(methylamino) methyl)pyridin-2-yl)-3-(1- methyl-1H-1,2,3-triazol-5-yl)- 2,7-naphthyridine-1,6- diamine | 402.3 | ¹H NMR (400 MHz, DMSO-d6) δ ppm 0.19- 0.28 (m, 1 H) 0.29-0.44 (m, 2 H) 0.50- 0.60 (m, 1 H) 0.87-0.98 (m, 1 H) 2.17 (s, 3 H) 2.66 (d, J = 8.53 Hz, 1H) 4.39 (s, 3 H) 6.93 (d, J = 5.77 Hz, 1 H) 7.26 (s, 1 H) 7.31 (s, 1 H) 7.35 (s, 2 H) 8.20- 8.25 (m, 2 H) 8.32 (s. 1 H) 9.30 (s, 1 H) 9.89 (s, 1 H); [α]24D = −38.0° (c = 1.0 mg/mL, MeOH) |
| 17 Enantiomer 1 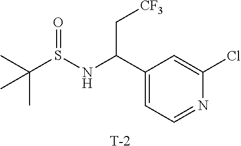 | 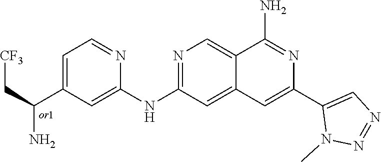 [SMILES: Cn1nncc1-c1cc2cc(Nc3cc([C@@H](N)CC(F)(F)F)ccn3)ncc2c(N)n1] N⁶-(4-(1-amino-3,3,3- trifluoropropyl)pyridin-2-yl)-3- (1-methyl-1H-1,2,3-triazol-5- yl)-2,7-naphthyridine-1,6- diamine | 430.2 | ¹H NMR (400 MHz, DMSO-d6) δ = 9.94 (s, 1H), 9.32 (s, 1H), 8.31 (S, 1H), 8.28- 8.23 (m, 2H), 7.37 (br s, 2H), 7.33 (s, 1H), 7.27 (s, 1H), 7.02 (d, J = 5.3 Hz, 1H), 4.39 (s, 3H), 4.05 (t, J = 6.8 Hz, 1H), 2.66-2.58 (m, 2H), 2.16 (br s, 2H); [α]24D = +14.0° (c = 1.0 mg/mL, MeOH) |
| 18 Enantiomer 2 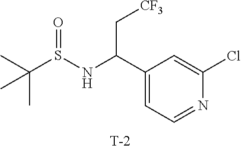 | 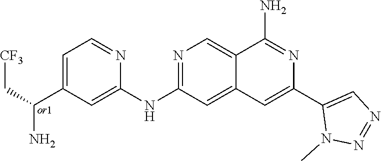 [SMILES: Cn1nncc1-c1cc2cc(Nc3cc([C@H](N)CC(F)(F)F)ccn3)ncc2c(N)n1] N⁶-(4-(1-amino-3,3,3- trifluoropropyl)pyridin-2-yl)-3- (1-methyl-1H-1,2,3-triazol-5- yl)-2,7-naphthyridine-1,6- diamine | 430.4 | 1H NMR (400 MHz, DMSO-d6) δ = 9.95 (s, 1H), 9.32 (s, 1H), 8.31 (S, 1H), 8.28- 8.22 (m, 2H), 7.35 (br d, J = 16.3 Hz, 3H), 7.27 (s, 1H), 7.02 (d, J = 5.0 Hz, 1H), 4.40 (s, 3H), 4.05 (t, J = 6.7 Hz, 1H), 2.65-2.58 (m, 2H), 2.20 (br s, 2H); [α]24D = −4.67° (c = 1.1 mg/mL, MeOH) |
| 19 Enantiomer 2 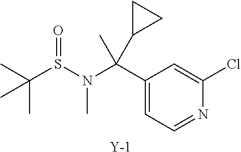 | 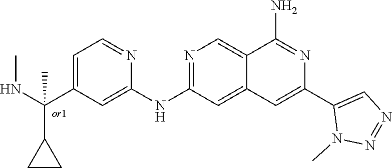 [SMILES: CN[C@@](C)(c1ccnc(Nc2cc3cc(-c4cnnn4C)nc(N)c3cn2)c1)C1CC1] N⁶-(4-(1-cyclopropyl-1- (methylamino)ethyl)pyridin- 2-yl)-3-(1-methyl-1H-1,2,3- triazol-5-yl)-2,7- naphthyridine-1,6-diamine | 416.3 | ¹H NMR (400 MHz, DMSO-d6) δ ppm 0.32- 0.46 (m, 4 H) 1.06 (quin, J = 6.96 Hz, 1 H) 1.14 (s, 3 H) 2.08 (s, 3 H) 4.39 (s, 3 H) 7.05 (dd, J = 5.40, 1.13 Hz, 1H) 7.26 (s, 1 H) 7.34 (s, 2 H) 7.47 (s, 1 H) 8.19- 8.26 (m, 2 H) 8.34 (s, 1 H) 9.30 (s, 1 H) 9.86 (s, 1 H); [α]24D = −8.0° (c = 1.0 mg/mL, MeOH) |
| 20 Enantiomer 1 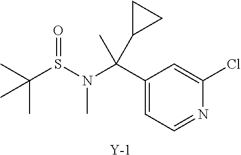 | 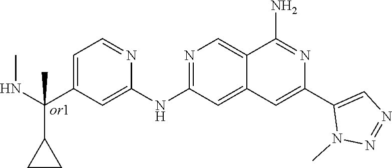 [SMILES: CN[C@](C)(c1ccnc(Nc2cc3cc(-c4cnnn4C)nc(N)c3cn2)c1)C1CC1] N⁶-(4-(1-cyclopropyl-1- (methylamino)ethyl)pyridin- 2-yl)-3-(1-methyl-1H-1,2,3- triazol-5-yl)-2,7- naphthyridine-1,6-diamine | 416.4 | ¹H NMR (400 MHz, DMSO-d6) δ ppm 0.31- 0.46 (m, 4 H) 1.06 (quin, J = 7.03 Hz, 1 H) 1.14 (s, 3 H) 2.08 (s, 3 H) 4.39 (s, 3 H) 7.05 (dd, J = 5.52, 1.00 Hz, 1H) 7.26 (s, 1 H) 7.34 (s, 2 H) 7.47 (s, 1 H) 8.21- 8.24 (m, 2 H) 8.34 (s, 1 H) 9.30 (s, 1 H) 9.86 (s, 1 H); [α]24D = +13.3° (c = 1.0 mg/mL, MeOH) |
| 21 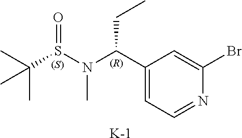 | 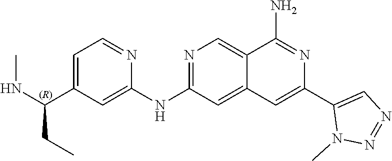 (R)-3-(1-methyl-1H-1,2,3- triazol-5-yl)-N⁶-(4-(1- (methylamino)propyl)pyridin- 2-yl)-2,7-naphthyridine-1,6- diamine | 390.1 | ¹H NMR (400 MHz, METHANOL- d4) δ 9.22 (s, 1H), 8.28 (d, J = 5.37 Hz, 1H), 8.26 (s, 1H), 8.11 (s, 1H), 7.26 (s, 1H), 7.19 (s, 1H), 6.93 (d, J = 5.45 Hz, 1H),4.43 (s, 3H), 3.41 (dd, J = 5.19, 8.69 Hz, 1H), 2.27 (s, 3H), 1.83- 1.93 (m, 1H), 1.65-1.76 (m, 1H), 0.87 (t, J = 7.44 Hz, 3H); [α]25D = +19.0° (c = 0.001 g/mL, MeOH) |
| 22 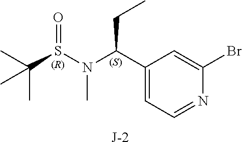 | 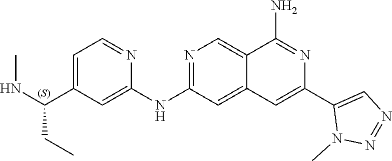 (S)-3-(1-methyl-1H-1,2,3- triazol-5-yl)-N⁶-(4-(1- (methylamino)propyl)pyridin- 2-yl)-2,7-naphthyridine-1,6- diamine | 390.4 | ¹H NMR (400 MHz, DMSO-d6) δ = 9.88 (s, 1H), 9.30 (s, 1H), 8.32 (s, 1H), 8.25- 8.20 (m, 2H), 7.35 (br s, 2H), 7.25 (d, J = 7.9 Hz, 2H), 6.87 (d, J = 5.0 Hz, 1H), 4.39 (s, 3H), 3.25 (br t, J = 6.4 Hz, 1H), 2.13 (s, 3H), 2.11- 2.04 (m, 1H), 1.72-1.60 (m, 1H), 1.58- 1.45 (m, 1H), 0.79 (t, J = 7.4 Hz, 3H); [α]23D = −3.0° (c = 0.001 g/mL, MeOH) |
| 23 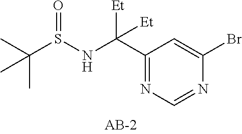 [SMILES: CCC(CC)(NS(=O)C(C)(C)C)c1cc(Br)ncn1] | 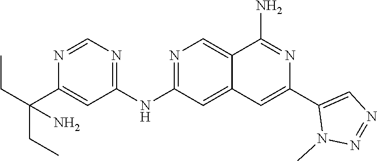 [SMILES: CCC(N)(CC)c1cc(Nc2cc3cc(-c4cnnn4C)nc(N)c3cn2)ncn1] N⁶-(6-(3-aminopentan-3- yl)pyrimidin-4-yl)-3-(1- methyl-1H-1,2,3-triazol-5-yl)- 2,7-naphthyridine-1,6- diamine | 405.3 | H NMR (400 MHz, METHANOL- d4) δ ppm 0.79-0.84 (m, 6 H) 1.79 (dd, J = 13.95, 7.44 Hz, 2 H) 1.94- 2.06 (m, 2 H) 4.44 (s, 3 H) 7.31 (s, 1 H) 7.41 (d, J = 0.88 Hz, 1 H) 8.12 (s, 1 H) 8.38 (s, 1 H) 8.77 (d, J = 1.00 Hz, 1 H) 9.27 (s, 1 H) |
| 24 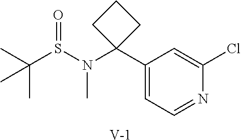 | 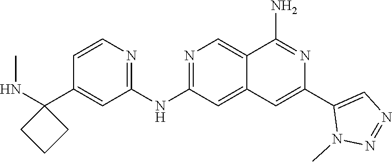 [SMILES: CNC1(c2ccnc(Nc3cc4cc(-c5cnnn5C)nc(N)c4cn3)c2)CCC1] 3-(1-methyl-1H-1,2,3-triazol- 5-y1)-N⁶-(4-(1- (methylamino)cyclobutyl) pyridin-2-yl)-2,7- naphthyridine- 1,6-diamine | 402.2 | ¹H NMR (400 MHz, DMSO-d6) δ ppm 1.72 (br d, J = 8.76 Hz, 1 H) 1.94 (s, 4 H) 2.13 (br d, J = 6.75 Hz, 2 H) 2.23 (br s, 2 H) 4.39 (s, 3 H) 6.93 (br d, J = 3.88 Hz, 1 H) 7.21-7.42 (m, 4 H) 8.14- 8.38 (m, 3 H) 9.30 (s, 1 H) 9.87 (s, 1 H) |
| 25 Enantiomer 1 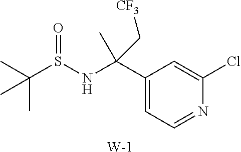 | 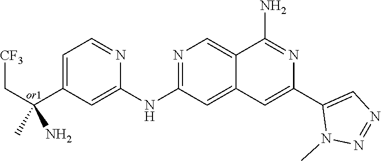 [SMILES: Cn1nncc1-c1cc2cc(Nc3cc([C@@](C)(N)CC(F)(F)F)ccn3)ncc2c(N)n1] N⁶-(4-(2-amino-4,4,4- trifluorobutan-2-yl)pyridin-2- yl)-3-(1-methyl-1H-1,2,3- triazol-5-yl)-2,7- naphthyridine-1,6-diamine | 444.3 | 1H NMR (400 MHz, METHANOL- d4) δ 9.22 (s, 1H), 8.25- 8.31 (m, 2H), 8.10 (s, 1H), 7.41 (s. 1H), 7.26 (s, 1H), 7.11 (br d, J = 5.52 Hz, 1H), 4.38- 4.47 (m,3H), 2.72-2.89 (m, 2H), 1.62 (s, 3H); [α]25D = −3.22° (c = 0.003 g/mL, MeOH) |
| 26 Enantiomer 2 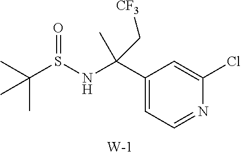 | 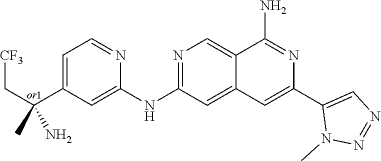 [SMILES: Cn1nncc1-c1cc2cc(Nc3cc([C@](C)(N)CC(F)(F)F)ccn3)ncc2c(N)n1] N⁶-(4-(2-amino-4,4,4- trifluorobutan-2-yl)pyridin-2- yl)-3-(1-methyl-1H-1,2,3- triazol-5-yl)-2,7- naphthyridine-1,6-diamine | 444.3 | ¹H NMR (400 MHz, METHANOL- d4) δ 9.22 (s, 1H), 8.28 (s, 1H), 8.28 (d, J = 5.51 Hz, 1H), 8.10 (s, 1H), 7.41 (s, 1H), 7.26 (s, 1H), 7.12 (d, J = 5.52 Hz, 1H), 4.43 (s, 3H), 2.71-2.88 (m, 2H), 1.62 (s, 3H); [α]25D = +2.0° (c = 0.003 g/mL, MeOH) |
| 27 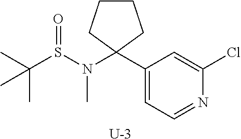 | 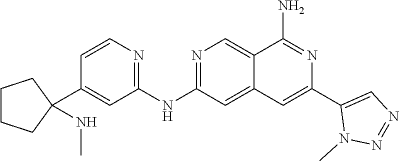 [SMILES: CNC1(c2ccnc(Nc3cc4cc(-c5cnnn5C)nc(N)c4cn3)c2)CCCC1] 3-(1-methyl-1H-1,2,3-triazol- 5-y1)-N⁶-(4-(1- (methylamino)cyclopentyl) pyridin-2-yl)-2,7-naphthyridine- 1,6-diamine | 416.4 | ¹H NMR (400 MHz, METHANOL- d4) δ ppm 9.22 (s, 1 H) 8.41 (d, J = 5.38 Hz, 1 H) 8.32 (br s, 1 H) 8.22 (s, 1 H) 8.08 (s. 1 H) 7.53 (s, 1 H) 7.23 (s, 1 H) 7.06 (dd, J = 5.50, 1.47 Hz, 1 H) 4.41 (s, 3 H) 2.48 (s, 3 H) 2.40- 2.46 (m, 2 H) 2.23-2.32 (m, 2 H) 1.95- 2.02 (m, 2 H) 1.82- 1.91 (m, 2 H) |
| 28 Enantiomer 1 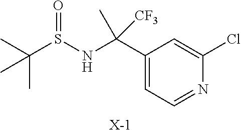 [SMILES: CC(C)(C)S(=O)NC(C)(c1ccnc(Cl)c1)C(F)(F)F ; "X-1"] | 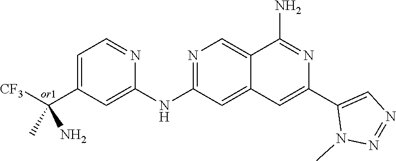 [SMILES: Cn1nncc1-c1cc2cc(Nc3cc([C@@](C)(N)C(F)(F)F)ccn3)ncc2c(N)n1] N⁶-(4-(2-amino-1,1,1- trifluoropropan-2-yl)pyridin-2- yl)-3-(1-methyl-1H-1,2,3- triazol-5-yl)-2,7- naphthyridine-1,6-diamine | 430.4 | ¹H NMR (500 MHz, DMSO-d6) δ = 10.04 (s, 1H), 9.38 (s, 1H), 8.37 (br s, 2H), 8.29 (s, 1H), 7.68 (s, 1H), 7.42 (br s, 2H), 7.34 (s, 1H), 7.23 (br d, J = 4.4 Hz, 1H), 4.45 (s, 3H), 1.63 (s, 3H); [α]25D = −30.0° (c = 1.1 mg/mL, MeOH) |
| 29 Enantiomer 2 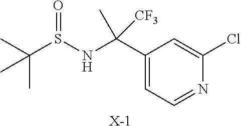 | 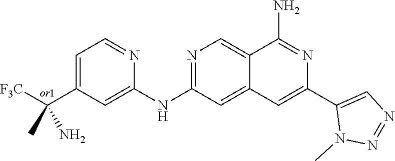 [SMILES: Cn1nncc1-c1cc2cc(Nc3cc([C@](C)(N)C(F)(F)F)ccn3)ncc2c(N)n1] N⁶-(4-(2-amino-1,1,1- trifluoropropan-2-yl)pyridin-2- yl)-3-(1-methyl-1H-1,2,3- triazol-5-yl)-2,7- naphthyridine-1,6-diamine | 430.4 | 1H NMR (400 MHz, DMSO-d6) δ = 9.99 (s, 1H), 9.32 (s, 1H), 8.31 (t, J = 2.7 Hz, 2H), 8.23 (s, 1H), 7.63 (s, 1H), 7.36 (s, 2H), 7.28 (s, 1H), 7.17 (d, J = 5.5 Hz, 1H), 4.40 (s, 3H), 2.63 (br s, 2H), 1.57 (s, 3H); [α]25D = +10.7° (c = 1.1 mg/mL, MeOH) |
| 30 Enantiomer 1 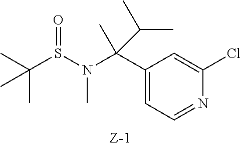 [SMILES: CC(C)C(C)(c1ccnc(Cl)c1)N(C)S(=O)C(C)(C)C] | 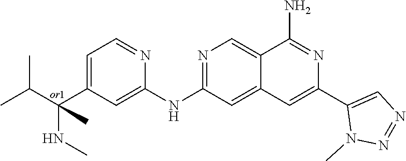 [SMILES: CN[C@@](C)(c1ccnc(Nc2cc3cc(-c4cnnn4C)nc(N)c3cn2)c1)C(C)C] 3-(1-methyl-1H-1,2,3-triazol- 5-yl)-N⁶-(4-(3-methyl-2- (methylamino)butan-2- yl)pyridin-2-yl)-2,7- naphthyridine-1,6-diamine | 418.4 | ¹H NMR (400 MHz, METHANOL- d4) δ 9.22 (s, 1H), 8.27 (s, 1H), 8.26 (d, J = 5.54 Hz, 1H), 8.10 (s, 1H), 7.25 (s, 2H), 7.00 (dd, J = 1.56, 5.44 Hz, 1H), 4.43 (s, 3H), 2.11 (s, 3H), 1.91-2.05 (m, 1H), 1.37-1.50 (m, 3H), 1.01 (d, J = 6.88 Hz, 3H), 0.75 (d, J = 6.75 Hz, 3H); [α]25D = −0.28° (c = 0.0024 g/mL, MeOH) |
| 31 Enantiomer 2 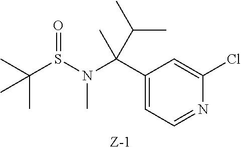 | 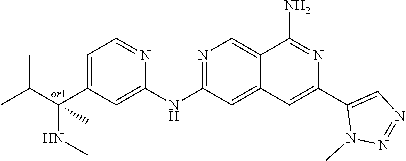 [SMILES: CN[C@](C)(c1ccnc(Nc2cc3cc(-c4cnnn4C)nc(N)c3cn2)c1)C(C)C] 3-(1-methyl-1H-1,2,3-triazol- 5-yl)-N⁶-(4-(3-methyl-2- (methylamino)butan-2- yl)pyridin-2-yl)-2,7- naphthyridine-1,6-diamine | 418.4 | ¹H NMR (400 MHz, METHANOL- d4) δ 9.21 (s, 1H), 8.26 (s, 1H), 8.27 (d, J = 5.45 Hz, 1H), 8.10 (s, 1H), 7.25 (d, J = 4.89 Hz, 2H), 7.00 (d, J = 5.60 Hz, 1H), 4.42 (s, 3H). 2.12 (s, 3H), 1.94-2.09 (m, 1H), 1.43 (s, 3H), 1.01 (d, J = 6.79 Hz, 3H), 0.75 (d, J = 6.79 Hz, 3H); [α]24D = +1.67° (c = 0.003 g/mL, MeOH) |
| 32 Enantiomer from A-3 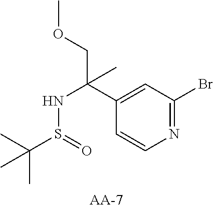 [SMILES: COCC(C)(NS(=O)C(C)(C)C)c1ccnc(Br)c1 ; "AA-7"] | 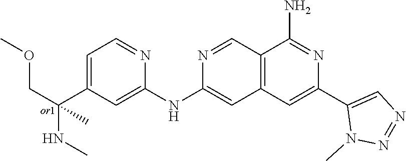 [SMILES: CN[C@@](C)(COC)c1ccnc(Nc2cc3cc(-c4cnnn4C)nc(N)c3cn2)c1] N⁶-(4-(1-methoxy-2- (methylamino)propan-2- yl)pyridin-2-yl)-3-(1-methyl- 1H-1,2,3-triazol-5-yl)-2,7- naphthyridine-1,6-diamine | 420.2 | ¹H NMR (600 MHz, DMSO-d6) δ 9.82 (s, 1H), 9.30 (s, 1H), 8.32 (s, 1H), 8.24-8.18 (m, 2H), 7.43- 7.36 (m, 1H), 7.31 (s, 2H), 7.25 (s, 1H), 7.00 (dd, J = 5.5, 1.5 Hz, 1H), 4.39 (s, 3H), 3.43 (d, J = 9.0 Hz, 1H), 3.37 (d, J = 9.0 Hz, 1H), 3.25 (s, 3H), 2.07 (s, 3H), 1.88 (s, 1H), 1.34 (s, 3H) |
| 33 Enantiomer from A-4 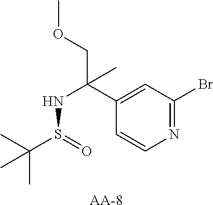 [SMILES: COCC(C)(N[S@@](=O)C(C)(C)C)c1ccnc(Br)c1 ; "AA-8"] | 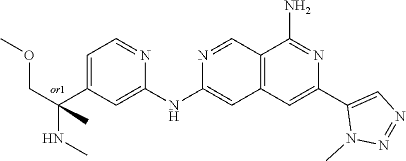 [SMILES: CN[C@](C)(COC)c1ccnc(Nc2cc3cc(-c4cnnn4C)nc(N)c3cn2)c1] N⁶-(4-(1-methoxy-2- (methylamino)propan-2- yl)pyridin-2-yl)-3-(1-methyl- 1H-1,2,3-triazol-5-yl)-2,7- naphthyridine-1,6-diamine | 420.2 | ¹H NMR (400 MHz, DMSO-d₆) δ 9.84 (s, 1H), 9.30 (s, 1H), 8.32 (s, 1H), 8.23 (d, J = 7.1 Hz, 2H), 7.43-7.38 (m, 1H), 7.32 (s, 2H), 7.26 (s, 1H), 7.00 (dd, J = 5.5, 1.6 Hz, 1H), 4.39 (s, 3H), 3.46-3.36 (m, 2H), 3.26 (s, 3H), 2.08 (s, 3H), 1.35 (s, 3H) |
| 34 Enantiomer 1 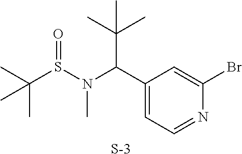 | 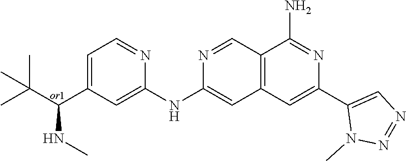 [SMILES: CN[C@H](c1ccnc(Nc2cc3cc(-c4cnnn4C)nc(N)c3cn2)c1)C(C)(C)C] N⁶-(4-(2,2-dimethyl-1- (methylamino)propyl)pyridin- 2-yl)-3-(1-methyl-1H-1,2,3- triazol-5-yl)-2,7- naphthyridine-1,6-diamine | 418.2 | ¹H NMR (600 MHz, DMSO) δ 9.58 (s, 1H), 9.30 (d, J = 1.0 Hz, 1H), 8.23-8.18 (m, 2H), 8.15 (s, 1H), 7.29 (d, J = 1.4 Hz, 1H), 7.22 (d, J = 0.8 Hz, 1H), 7.13 (s, 2H), 6.85 (dd, J = 5.2, 1.4 Hz, 1H), 4.38 (s, 3H), 3.87 (s, 3H), 2.15 (s, 3H), 1.88 (s, 1H), 0.91 (s, 9H); [α]22D = −50.1° (c = 0.2 g/100 mL, MeOH) |
| 35 Enantiomer 2 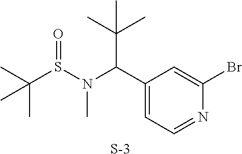 | 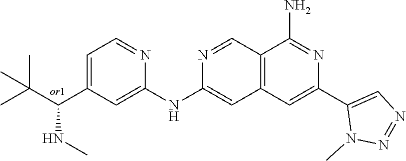 [SMILES: CN[C@@H](c1ccnc(Nc2cc3cc(-c4cnnn4C)nc(N)c3cn2)c1)C(C)(C)C] N⁶-(4-(2,2-dimethyl-1- (methylamino)propyl)pyridin- 2-yl)-3-(1-methyl-1H-1,2,3- triazol-5-yl)-2,7- naphthyridine-1,6-diamine | 418.2 | ¹H NMR (600 MHz, DMSO) δ 9.59 (s, 1H), 9.27 (d, J = 0.9 Hz, 1H), 8.19 (d, J = 5.2 Hz, 1H), 8.17 (s, 1H), 8.11 (s, 1H), 7.28 (s, 1H), 7.19 (s, 1H), 7.11 (s, 2H), 6.85 (d, J = 5.3 Hz, 1H), 4.34 (s, 3H). 3.84 (s, 3H), 2.15 (s, 3H), 0.89 (s, 9H); [α]22D = +48.0° (c = 0.3 g/100 mL, MeOH) |

Example 36 was in a similar fashion to Example 5 using 0-4 in place of G-11 in Step 1. No

| Example No./ | | MS | |
|---|---|---|---|
| Intermediates | Structure/Name | [M + 1] | NMR |
| 36 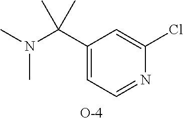 | 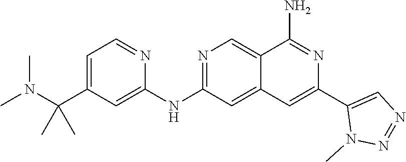 [SMILES: CN(C)C(C)(C)c1ccnc(Nc2cc3cc(-c4cnnn4C)nc(N)c3cn2)c1] N⁶-(4-(2- (dimethylamino)propan-2- yl)pyridin-2-yl)-3-(1-methyl- 1H-1,2,3-triazol-5-yl)-2,7- naphthyridine-1,6-diamine | 404.4 | ¹H NMR (400 MHz, DMSO- d6) δ ppm 1.28 (s, 6 H) 2.12 (s, 6 H) 4.30-4.50 (m, 3 H) 7.04 (dd, J = 5.38, 1.22 Hz, 1 H) 7.27 (s, 1 H) 7.34 (br s, 2 H) 7.45 (s, 1H) 8.15-8.30 (m, 2 H) 8.36 (s, 1 H) 9.30 (s, 1 H) 9.84 (s, 1 H) |

Example 37 and Example 38 were made in a similar fashion to Example 5 using the appropriate chloro-pyridine and C₁ in place of B3 in Step 1.

| Example No./ | | MS | |
|---|---|---|---|
| Intermediates | Structure/Name | [M + 1] | NMR |
| 37 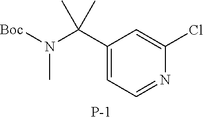 | 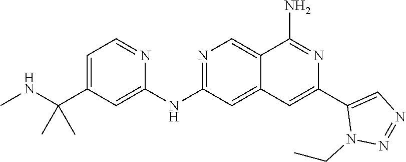 [SMILES: CCn1nncc1-c1cc2cc(Nc3cc(C(C)(C)NC)ccn3)ncc2c(N)n1] 3-(1-ethyl-1H-1,2,3-triazol-5-yl)- N⁶-(4-(2-(methylamino)propan- 2-yl)pyridin-2-yl)-2,7- naphthyridine-1,6-diamine | 404.4 | ¹H NMR (400 MHz, METHANOL- d4) δ = 9.22 (s, 1H), 8.28 (d, J = 5.5 Hz, 1H), 8.24 (s, 1H), 8.08 (s, 1H), 7.33 (d, J = 0.9 Hz, 1H), 7.24 (s, 1H), 7.05 (dd, J = 1.5, 5.5 Hz, 1H), 4.96 (q, J = 7.3 Hz, 2H), 2.17 (s, 3H), 1.53-1.47 (m, 9H) |
| 38 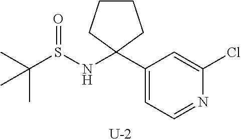 | 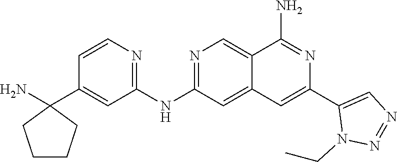 [SMILES: CCn1nncc1-c1cc2cc(Nc3cc(C4(N)CCCC4)ccn3)ncc2c(N)n1] N⁶-(4-(1- aminocyclopentyl)pyridin-2-yl)- 3-(1-ethyl-1H-1,2,3-triazol-5-yl)- 2,7-naphthyridine-1,6-diamine | 416.2 | ¹H NMR (400 MHz, DMSO- d6) δ = 9.83 (s, 1H), 9.31 (s, 1H), 8.33 (s, 1H), 8.25- 8.18 (m, 2H), 7.45 (s, 1H), 7.33 (br s, 2H), 7.26 (s, 1H), 7.05 (dd, J = 1.5, 5.4 Hz, 1H), 4.94 (q, J = 7.2 Hz, 2H), 2.23-1.97 (m, 2H), 1.93- 1.81 (m, 4H), 1.79-1.69 (m, 4H), 1.41 (t, J = 7.2 Hz, 3H) |

### MASTL Enzyme Kinase Assay

MASTL enzyme kinase assay was used to measure the inhibition constant (Kᵢ) of Examples of the present invention.

Inhibition of MASTL was measured using a CHEF PhosphoSens® method (Lukovic et al, 2008) at Pfizer Inc (San Diego, CA) or HD Biosciences inc, Shaghai. Full-length human recombinant N-term FLAG-tagged MASTL protein (construct LJIC-3077G2), activated by CDK1, was produced at Pfizer Inc by expression in baculovirus-infected insect cells. All reagents were purchased from Sigma-Aldrich, Inc (St. Louis, MO), unless specified otherwise. Tween™ 20 detergent (10% Solution) was purchased from EMD Millipore Corp (Billerica, MA). The CHEF phosphoacceptor peptide substrate (AQT0693) was purchased from AssayQuant technologies, Inc (Marlboro, MA). Reactions were conducted in 384-well plates at room temperature in the presence of 11-dose 3-fold serially diluted compounds (0.05-3000 nM). MASTL reactions contained 3 nM MASTL enzyme, 10 μM AQT0693 (CHEF peptide substrate), 1 mM DTT, 0.01% Tween™ 20 and 15 mM MgCl₂ in 50 mM HEPES, pH 7.15. The reactions contained 1 mM ATP and were initiated by the addition of enzyme. Initial reaction velocities were determined by following the fluorescence (λₑₓ=360 nm, λₑₘ=485 nm) of appropriate CHEF peptide substrate for 120 minutes in an end-point mode in a SpectraMax Paradigm Microplate Reader (Molecular Devices, LLC. San Jose, California). The extents of reactions were estimated to be <20%. Inhibitors were demonstrated to be competitive with ATP by enzyme kinetics studies. The Kᵢ were calculated by fitting the fluorescence based fractional initial velocities to the Morrison equation for tight-binding competitive inhibition (Morrison, 1969; Copeland, 2013) using ABase software (IDBS, London, United Kingdom) and experimentally measured apparent Michaelis constant for ATP (Kₘ^{app}=30 μM) for MASTL. The K, values were reported as geometric means with Standard Deviations based on at least 3 independent determinations unless otherwise noted.

| Example | MASTL |
|---|---|
| No. | Ki (nM) |
| 1 | 0.032 |
| 2 | 0.12 |
| 3 | 0.36 |
| 4* | 0.47 |
| 5 | 0.033 |
| 6 | 0.076 |
| 7 | 0.063 |
| 8 | 0.021 |
| 9 | 0.03 |
| 10 | 0.034 |
| 11 | 0.018 |
| 12 | 0.029 |
| 13 | 0.065 |
| 14 | 0.098 |
| 15 | 0.038 |
| 16 | 0.029 |
| 17 | 0.12 |
| 18 | 0.088 |
| 19 | 0.047 |
| 20 | 0.049 |
| 21 | 0.055 |
| 22 | 0.02 |
| 23 | 0.057 |
| 24 | 0.089 |
| 25 | 0.078 |
| 26 | 0.075 |
| 27 | 0.073 |
| 28 | 0.093 |
| 29 | 0.17 |
| 30 | 0.036 |
| 31 | 0.03 |
| 32 | 0.087 |
| 33 | 0.25 |
| 34 | 0.23 |
| 35 | 0.32 |
| 36 | 0.061 |
| 37 | 0.059 |
| 38 | 0.019 |

*denotes n = 2

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

We claim:

1. A compound of Formula (I): 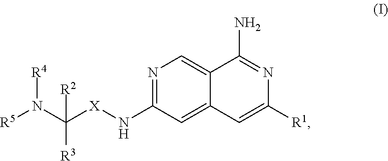
or a pharmaceutically acceptable salt thereof, wherein:
X is 5-10 membered heteroaryl comprising one, two, or three heteroatoms selected from the group consisting of O, N and S, wherein X is optionally substituted with one or two substituents independently selected from the group consisting of halogen, C₁-C₆ alkyl, C₃-C₆ cycloalkyl, C₁-C₆ fluoroalkyl, and C₁-C₆ alkoxy;
R¹ is 5-10 membered heteroaryl or 4-10 membered heterocycloalkyl, wherein R¹ comprises one, two, three, or four heteroatoms selected from the group consisting of O, N and S, and R¹ is optionally substituted with one, two, three, or four substituents independently selected from the group consisting of halogen, C₁-C₆ alkyl, C₃-C₆ cycloalkyl, C₁-C₆ fluoroalkyl, and oxo (═O);
R² and R³ are each independently selected from the group consisting of H, C₁-C₆ alkyl, C₃-C₆ cycloalkyl, C₂-C₆ alkoxyalkyl, and C₁-C₆ fluoroalkyl, or R² and R³ are taken together with the carbon atom to which they are attached to form a 3-8 membered cycloalkyl, or one of R² and R³ is taken together with the carbon atom to which they are attached to, and together with X to form a 3-8 membered ring that is fused to X; and
R⁴ and R⁵ are each independently selected from the group consisting of H, C₁-C₆ alkyl, C₃-C₆ cycloalkyl, and C₁-C₆ fluoroalkyl, or R⁴ and R⁵ are taken together with the N atom to which they are attached to form a 3-8 membered heterocycloalkyl, or one of R⁴ and R⁵ is taken together with the N atom to which they are attached to, and together with X to form a 5-8 membered ring that is fused to X,
wherein one of R² and R³ with the carbon atom to which they are attached to, and one of R⁴ and R⁵ with the N atom to which they are attached to, are optionally taken together to form a 3-8 membered heterocycloalkyl.

2. A compound of claim 1, or a pharmaceutically acceptable salt thereof, wherein X is 6-membered heteroaryl comprising one or two N atoms.

3. A compound of claim 2, or a pharmaceutically acceptable salt thereof, wherein X is pyridinyl or pyrimidinyl.

4. A compound of claim 3, or a pharmaceutically acceptable salt thereof, wherein X is pyridinyl.

5. A compound of any one of claims 1 to 4, or a pharmaceutically acceptable salt thereof, wherein the compound has Formula (I-a):

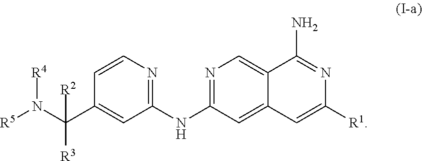

6. A compound of any one of claims 1 to 5, or a pharmaceutically acceptable salt thereof, wherein R¹ is 5-6 membered heteroaryl or 5-6 membered heterocycloalkyl comprising one, two, or three heteroatoms selected from the group consisting of O, N and S, wherein R¹ is optionally substituted with one or two substituents independently selected from the group consisting of halogen, C₁-C₃ alkyl, C₃-C₆ cycloalkyl, C₁-C₃ fluoroalkyl, and oxo (═O).

7. A compound of claim 6, or a pharmaceutically acceptable salt thereof, wherein R¹ is selected from the group consisting of:

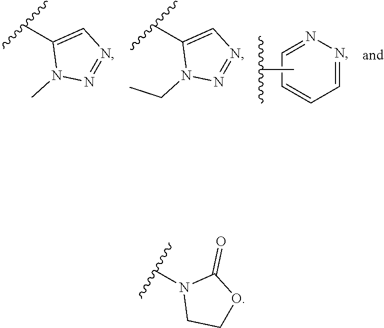

8. A compound of claim 7, or a pharmaceutically acceptable salt thereof, wherein R¹ is selected from the group consisting of:

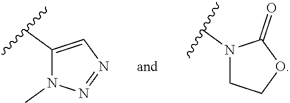

9. A compound of claim 8, or a pharmaceutically acceptable salt thereof, wherein R¹ is:

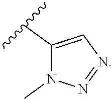 [SMILES: *c1cnnn1C]

10. A compound of claim 8, or a pharmaceutically acceptable salt thereof, wherein R¹ is:

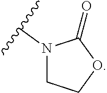 [SMILES: *N1CCOC1=O]

11. A compound of any one of claims 1 to 10, or a pharmaceutically acceptable salt thereof, wherein R² and R³ are each independently selected from the group consisting of H, C₁-C₃ alkyl, C₃-C₆ cycloalkyl, C₂-C₃ alkoxyalkyl, and C₁-C₃ fluoroalkyl, or R² and R³ are taken together with the carbon atom to which they are attached to form a 3-6 membered cycloalkyl, or one of R² and R³ are taken together with the carbon atom to which they are attached to, and together with X to form a 5-6 membered ring that is fused to X.

12. A compound of claim 11, or a pharmaceutically acceptable salt thereof, wherein R² and R³ are each independently selected from the group consisting of H, methyl, ethyl, isopropyl, cyclopropyl, tert-butyl, —CF₃, —CH₂—CF₃, and —CH₂—O—CH₃, or R² and R³ are taken together with the carbon atom to which they are attached to form a 3-5 membered cycloalkyl, or one of R² and R³ are taken together with the carbon atom to which they are attached to, and together with X to form a 5-6 membered ring that is fused to X.

13. A compound of claim 12, or a pharmaceutically acceptable salt thereof, wherein R² and R³ are each independently selected from the group consisting of H, methyl, ethyl, isopropyl, cyclopropyl, tert-butyl, —CF₃, —CH₂—CF₃, and —CH₂—O—CH₃, or R² and R³ are taken together with the carbon atom to which they are attached to form a 3-5 membered cycloalkyl.

14. A compound of any one of claims 1 to 13, or a pharmaceutically acceptable salt thereof, wherein R⁴ and R⁵ are each independently H or methyl.

15. A compound of any one of claims 1 to 14, or a stereoisomer thereof, or a pharmaceutically acceptable saft of the compound or the stereoisomer of the compound thereof, wherein the compound is selected from the group consisting of:

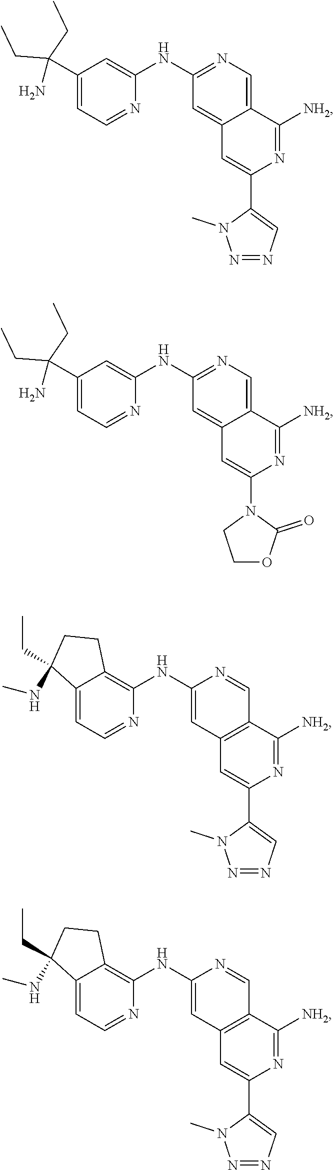 [SMILES: CCC(N)(CC)c1ccnc(Nc2cc3cc(-c4cnnn4C)nc(N)c3cn2)c1 ; CCC(N)(CC)c1ccnc(Nc2cc3cc(N4CCOC4=O)nc(N)c3cn2)c1 ; CC[C@]1(NC)CCc2c1ccnc2Nc1cc2cc(-c3cnnn3C)nc(N)c2cn1 ; CC[C@@]1(NC)CCc2c1ccnc2Nc1cc2cc(-c3cnnn3C)nc(N)c2cn1]

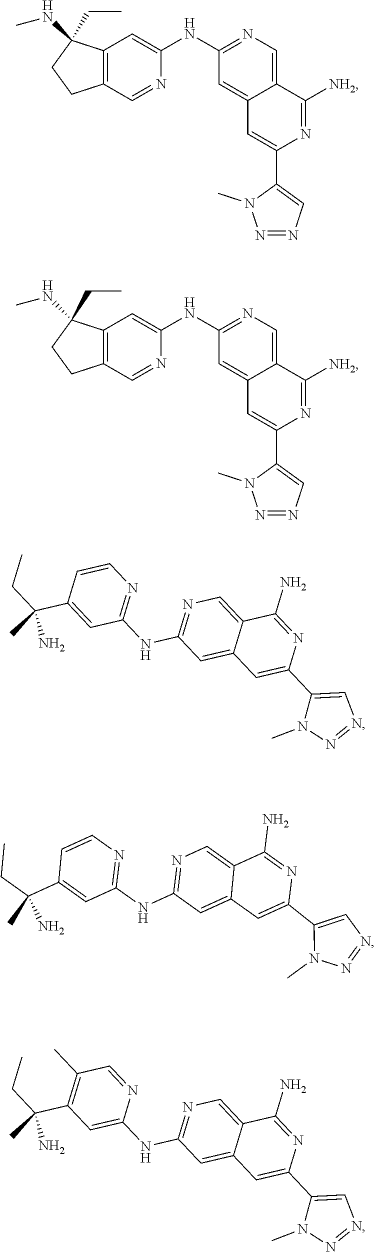 [SMILES: CC[C@@]1(NC)CCc2cnc(Nc3cc4cc(-c5cnnn5C)nc(N)c4cn3)cc21 ; CC[C@]1(NC)CCc2cnc(Nc3cc4cc(-c5cnnn5C)nc(N)c4cn3)cc21 ; CC[C@@](C)(N)c1ccnc(Nc2cc3cc(-c4cnnn4C)nc(N)c3cn2)c1 ; CC[C@@](C)(N)c1ccnc(Nc2cc3cc(-c4cnnn4C)nc(N)c3cn2)c1 ; CC[C@@](C)(N)c1cc(Nc2cc3cc(-c4cnnn4C)nc(N)c3cn2)ncc1C]

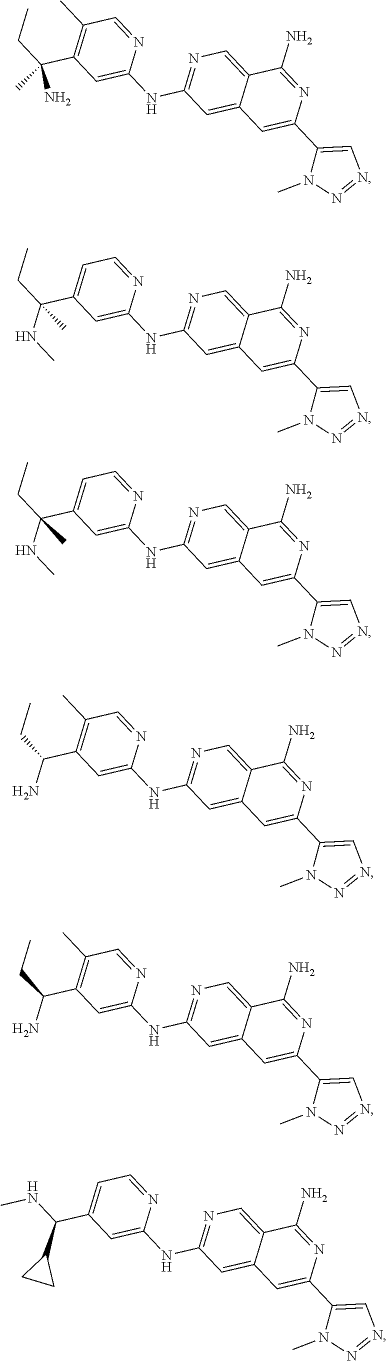 [SMILES: CC[C@](C)(N)c1cc(Nc2cc3cc(-c4cnnn4C)nc(N)c3cn2)ncc1C ; CC[C@](C)(NC)c1ccnc(Nc2cc3cc(-c4cnnn4C)nc(N)c3cn2)c1 ; CC[C@@](C)(NC)c1ccnc(Nc2cc3cc(-c4cnnn4C)nc(N)c3cn2)c1 ; CC[C@@H](N)c1cc(Nc2cc3cc(-c4cnnn4C)nc(N)c3cn2)ncc1C ; CC[C@H](N)c1cc(Nc2cc3cc(-c4cnnn4C)nc(N)c3cn2)ncc1C ; CN[C@@H](c1ccnc(Nc2cc3cc(-c4cnnn4C)nc(N)c3cn2)c1)C1CC1]

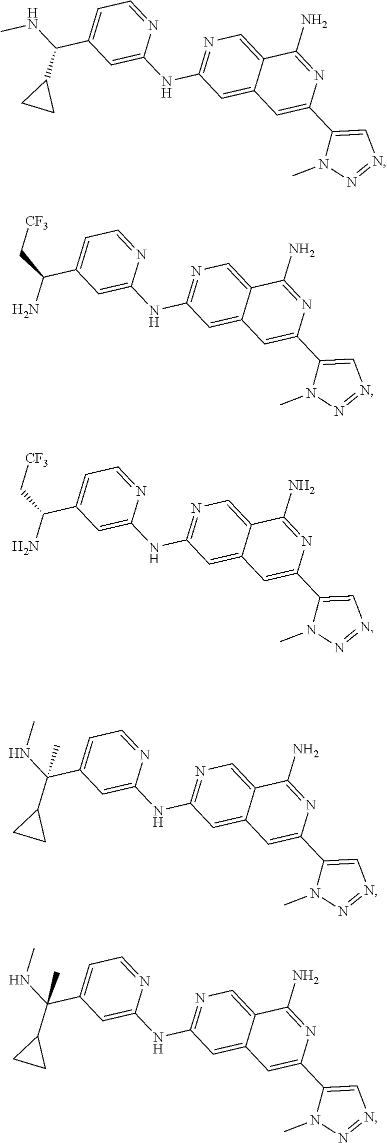 [SMILES: CN[C@H](c1ccnc(Nc2cc3cc(-c4cnnn4C)nc(N)c3cn2)c1)C1CC1 ; Cn1nncc1-c1cc2cc(Nc3cc([C@@H](N)CC(F)(F)F)ccn3)ncc2c(N)n1 ; Cn1nncc1-c1cc2cc(Nc3cc([C@H](N)CC(F)(F)F)ccn3)ncc2c(N)n1 ; CN[C@@](C)(c1ccnc(Nc2cc3cc(-c4cnnn4C)nc(N)c3cn2)c1)C1CC1 ; CN[C@](C)(c1ccnc(Nc2cc3cc(-c4cnnn4C)nc(N)c3cn2)c1)C1CC1]

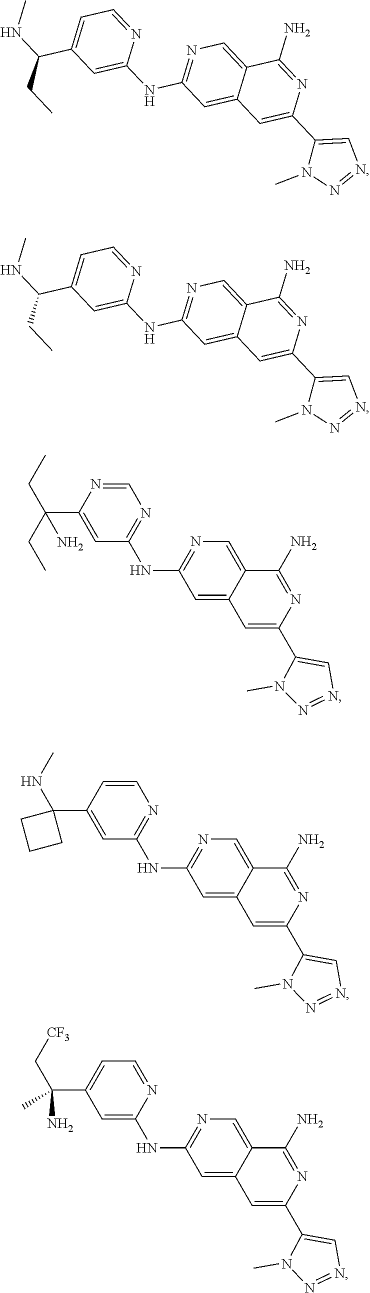 [SMILES: CC[C@@H](NC)c1ccnc(Nc2cc3cc(-c4cnnn4C)nc(N)c3cn2)c1 ; CC[C@H](NC)c1ccnc(Nc2cc3cc(-c4cnnn4C)nc(N)c3cn2)c1 ; CCC(N)(CC)c1cc(Nc2cc3cc(-c4cnnn4C)nc(N)c3cn2)ncn1 ; CNC1(c2ccnc(Nc3cc4cc(-c5cnnn5C)nc(N)c4cn3)c2)CCC1 ; Cn1nncc1-c1cc2cc(Nc3cc([C@@](C)(N)CC(F)(F)F)ccn3)ncc2c(N)n1]

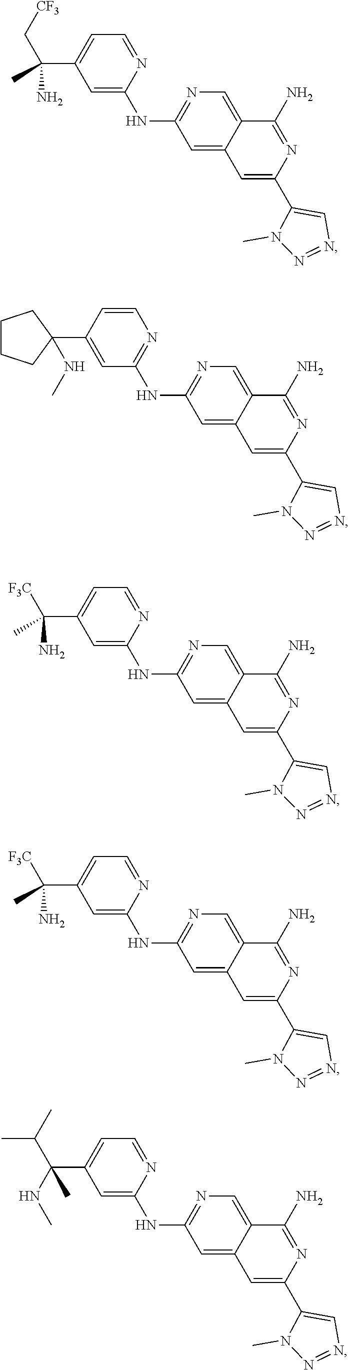 [SMILES: Cn1nncc1-c1cc2cc(Nc3cc([C@](C)(N)CC(F)(F)F)ccn3)ncc2c(N)n1 ; CNC1(c2ccnc(Nc3cc4cc(-c5cnnn5C)nc(N)c4cn3)c2)CCCC1 ; Cn1nncc1-c1cc2cc(Nc3cc([C@@](C)(N)C(F)(F)F)ccn3)ncc2c(N)n1 ; Cn1nncc1-c1cc2cc(Nc3cc([C@](C)(N)C(F)(F)F)ccn3)ncc2c(N)n1 ; CN[C@@](C)(c1ccnc(Nc2cc3cc(-c4cnnn4C)nc(N)c3cn2)c1)C(C)C]

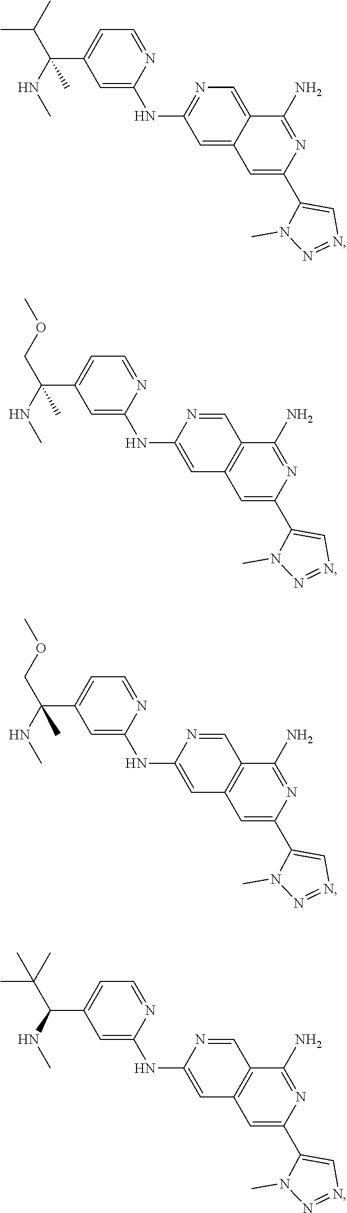 [SMILES: CN[C@](C)(c1ccnc(Nc2cc3cc(-c4cnnn4C)nc(N)c3cn2)c1)C(C)C ; CN[C@@](C)(COC)c1ccnc(Nc2cc3cc(-c4cnnn4C)nc(N)c3cn2)c1 ; CN[C@](C)(COC)c1ccnc(Nc2cc3cc(-c4cnnn4C)nc(N)c3cn2)c1 ; CN[C@H](c1ccnc(Nc2cc3cc(-c4cnnn4C)nc(N)c3cn2)c1)C(C)(C)C]

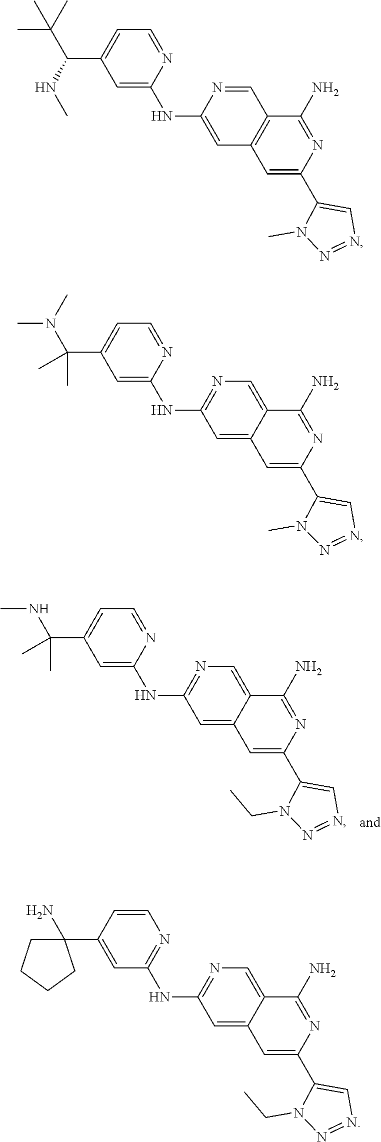 [SMILES: CN[C@@H](c1ccnc(Nc2cc3cc(-c4cnnn4C)nc(N)c3cn2)c1)C(C)(C)C ; CN(C)C(C)(C)c1ccnc(Nc2cc3cc(-c4cnnn4C)nc(N)c3cn2)c1 ; CCn1nncc1-c1cc2cc(Nc3cc(C(C)(C)NC)ccn3)ncc2c(N)n1 ; CCn1nncc1-c1cc2cc(Nc3cc(C4(N)CCCC4)ccn3)ncc2c(N)n1]

16. A compound that is 10-(4-(3-aminopentan-3-yl)pyridin-2-yl)-3-(1-methyl-1H-1,2,3-triazol-5-yl)-2,7-naphthyridine-1,6-diamine, having the structure: 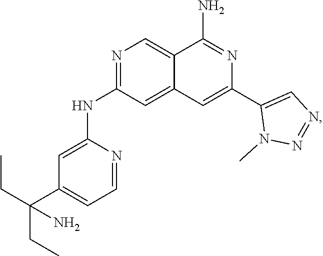 [SMILES: CCC(N)(CC)c1ccnc(Nc2cc3cc(-c4cnnn4C)nc(N)c3cn2)c1]
or a pharmaceutically acceptable salt thereof.

17. A compound that is N⁶-(4-(3-aminopentan-3-yl)pyridin-2-yl)-3-(1-methyl-1H-1,2,3-triazol-5-yl)-2,7-naphthyridine-1,6-diamine, having the structure:

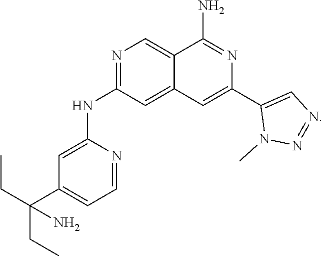 [SMILES: CCC(N)(CC)c1ccnc(Nc2cc3cc(-c4cnnn4C)nc(N)c3cn2)c1]

18. A pharmaceutically acceptable saft of a compound, wherein the compound is N⁶-(4-(3-aminopentan-3-yl)pyridin-2-yl)-3-(1-methyl-1H-1,2,3-triazol-5-yl)-2,7-naphthyridine-1,6-diamine, having the structure:

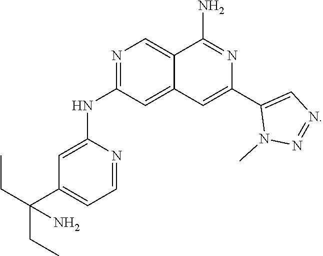 [SMILES: CCC(N)(CC)c1ccnc(Nc2cc3cc(-c4cnnn4C)nc(N)c3cn2)c1]

19. A compound that is 3-(1-amino-6-((4-(3-aminopentan-3-yl)pyridin-2-yl)amino)-2,7-naphthyridin-3-yl)oxazolidin-2-one, having the structure: 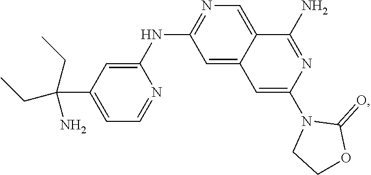 [SMILES: CCC(N)(CC)c1ccnc(Nc2cc3cc(N4CCOC4=O)nc(N)c3cn2)c1]
or a pharmaceutically acceptable salt thereof.

20. A compound that is 3-(1-amino-6-((4-(3-aminopentan-3-y)pyridin-2-yl)amino)-2,7-naphthyridin-3-yl)oxazolidin-2-one, having the structure:

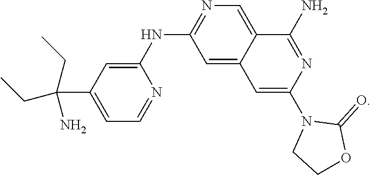 [SMILES: CCC(N)(CC)c1ccnc(Nc2cc3cc(N4CCOC4=O)nc(N)c3cn2)c1]

21. A pharmaceutically acceptable saft of a compound, wherein the compound is 3-(1-amino-6-((4-(3-aminopentan-3-yl)pyridin-2-yl)amino)-2,7-naphthyridin-3-yl)oxazolidin-2-one, having the structure:

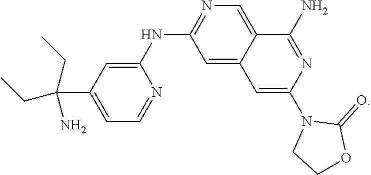 [SMILES: CCC(N)(CC)c1ccnc(Nc2cc3cc(N4CCOC4=O)nc(N)c3cn2)c1]

22. A pharmaceutical composition comprising a compound of any one of claims 1 to 21, or a pharmaceutically acceptable salt thereof, and at least one pharmaceutically acceptable excipient.

23. A method for treating cancer, comprising administering to a subject in need thereof a therapeutically effective amount of a compound of any one of claims 1 to 21, or a pharmaceutically acceptable salt thereof.

24. A method for treating cancer, comprising administering to a subject in need thereof a therapeutically effective amount of a compound of any one of claims 1 to 21, or a pharmaceutically acceptable salt thereof, as a single agent.

25. A method for treating cancer, comprising administering to a subject in need thereof a therapeutically effective amount of a compound of any one of claims 1 to 21, or a pharmaceutically acceptable salt thereof, and further comprising administering a therapeutically effective amount of an additional anticancer therapeutic agent.

26. A method for treating cancer of any one of claims 23 to 25, wherein the cancer is breast cancer, colon cancer, head and neck squamous cell carcinoma, non-small cell lung cancer (NSCLC), gastric cancer, pancreatic cancer, prostate cancer, or thyroid cancer.

27. A method for treating cancer according to claim 26, wherein the cancer is breast cancer, non-small cell lung cancer (NSCLC), or pancreatic cancer.

28. A compound of any one of claims 1 to 21, or a pharmaceutically acceptable salt thereof, for use as a medicament.

29. A compound of any one of claims 1 to 21, or a pharmaceutically acceptable salt thereof, for use in the treatment of cancer.

30. A compound for use in the treatment of cancer according to claim 29, wherein the cancer is breast cancer, colon cancer, head and neck squamous cell carcinoma, non-small cell lung cancer (NSCLC), gastric cancer, pancreatic cancer, prostate cancer, or thyroid cancer.

31. A compound for use in the treatment of cancer according to claim 30, wherein the cancer is breast cancer, non-small cell lung cancer (NSCLC), or pancreatic cancer.

32. Use of a compound of any one of claims 1 to 21, or a pharmaceutically acceptable salt thereof, for the manufacture of a medicament for the treatment of cancer.

33. Use of a compound, or a pharmaceutically acceptable salt thereof, for the manufacture of a medicament for the treatment of cancer according to claim 32, wherein the cancer is breast cancer, colon cancer, head and neck squamous cell carcinoma, non-small cell lung cancer (NSCLC), gastric cancer, pancreatic cancer, prostate cancer, or thyroid cancer.

34. Use of a compound, or a pharmaceutically acceptable salt thereof, for the manufacture of a medicament for the treatment of cancer according to claim 33, wherein the cancer is breast cancer, non-small cell lung cancer (NSCLC), or pancreatic cancer.

35. A method for the treatment of a disorder mediated by inhibition of MASTL receptor in a subject, comprising administering to the subject in need thereof a compound of any one of claims 1 to 21, or a pharmaceutically acceptable salt thereof, in an amount that is effective for treating the disorder.

36. A pharmaceutical combination comprising a compound of any one of claims 1 to 21, or a pharmaceutically acceptable salt thereof, and at least one additional therapeutic agent or a pharmaceutically acceptable salt thereof.

37. A pharmaceutical composition comprising the pharmaceutical combination of claim 36 and at least one excipient.
